(19) United States
(12) Patent Application Publication
(10) Pub. No.: US 2024/0282221 A1
(43) Pub. Date: Aug. 22, 2024

(54) APPARATUS, SYSTEM, AND METHOD FOR PATIENT-SPECIFIC MODELS

(71) Applicant: Treace Medical Concepts, Inc., Ponte Vedra, FL (US)

(72) Inventors: Adam D. Perler, St. Petersburg, FL (US); James Q. Spitler, Winter Garden, FL (US)

(21) Appl. No.: 18/444,352

(22) Filed: Feb. 16, 2024

Related U.S. Application Data
(60) Provisional application No. 63/446,782, filed on Feb. 17, 2023.

(51) Int. Cl. G09B 23/34; A61B 34/10

(52) U.S. Cl. CPC G09B 23/34; A61B 34/10; A61B 2034/104; A61B 2034/105; A61B 2034/108

(57) ABSTRACT

An apparatus, system, and method are disclosed for providing a three-dimensional (3D) physical model for a surgical procedure. In some aspects, the apparatus, system, and/or method may include generating a computer bone model of deformed osseous anatomy of a patient based on medical imaging data of the deformed osseous anatomy. Also, the apparatus may include fabricating a 3D physical bone model of the deformed osseous anatomy based on the computer bone model. Furthermore, the apparatus may include generating a computer instrument model of an instrument configured for use in a surgical procedure to remediate the deformed osseous anatomy. In addition, the apparatus may include fabricating the instrument based on the computer instrument model. Moreover, the apparatus may include providing the 3D physical bone model and the instrument to a user.

### CROSS-REFERENCE TO RELATED APPLICATIONS

This application claims the benefit of and priority to U.S. Provisional Application No. 63/446,782, filed Feb. 17, 2023, which is hereby incorporated by reference in its entirety.

### TECHNICAL FIELD

The present disclosure relates to surgical devices, systems, instruments, and methods. More specifically, the present disclosure relates to patient-specific guides, implants, instruments, and/or methods of designing and using the same.

### BACKGROUND

Various bone conditions may be corrected using surgical procedures, in which one or more tendons, ligaments, and/or bones may be cut, replaced, repositioned, reoriented, reattached, fixated and/or fused. These surgical procedures require the surgeon to properly locate, position, and/or orient one or more osteotomy cuts, fixation guides, fixators, bone tunnels, points of attachment for ends of grafts or soft tissue and the like. Determining and locating an optimal or desired location and trajectory for one or more steps of the surgical procedures can be challenging, given conventional techniques and instruments.

Hallux valgus or bunion conditions can be a source of discomfort, pain, and inconvenience for patients. Among a variety of different approaches for dealing with hallux valgus or bunion conditions, a Lapidus arthrodesis or simply Lapidus procedure is a common surgical procedure to address this condition. Advancements in medical imaging, preoperative planning, modeling, and the like have led to improvements that help surgeons execute a Lapidus surgical procedure. More and more surgeons are using preoperative planning software and/or models to prepare for a surgical procedure, including virtual rehearsal, and the like. However, the convenience and precision offered by software tools, graphical user interfaces, augmented reality, or virtual reality are not the same as the tangible three-dimensional experience of performing a surgical procedure. During an examination or surgical procedure, a surgeon can handled and feel the bones and other anatomy of a patient and get important feedback on how the parts of the anatomy move and interact with each other.

Three-dimensional models of patient anatomy exist today. However, a surgeon's preparation can be enhanced if they can hold, review, examine, manipulate, and/or rehearse with a physical model that includes a deformity that matches the deformity the patient presents with. Furthermore, a surgeon's preparation can be enhanced if the surgeon has a three-dimensional physical model of patient anatomy at one or more stages of a surgical procedure, including a postoperative condition of the anatomy. What is needed is a solution that facilitates implementation of a preoperative plan or modeled correction and/or modeled procedure before, during, and after the actual surgical procedure by way of three-dimensional physical models of patient anatomy. The present disclosure provides such a solution.

### SUMMARY

The various apparatuses, devices, systems and methods of the present disclosure have been developed in response to the present state of the art, and in particular, in response to the problems and needs in the art that have not yet been fully solved by currently available apparatuses, devices, systems, and/or methods.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In one general aspect, a method may include generating a computer model of deformed osseous anatomy of a patient based on medical imaging data of the deformed osseous anatomy. The method may also include fabricating a 3D physical bone model of the deformed osseous anatomy based on the computer model. The method may furthermore include providing the 3D physical bone model to a user. Other embodiments of this aspect include corresponding computer systems, apparatus, and computer programs recorded on one or more computer storage devices, each configured to perform the actions of the methods.

Implementations may include one or more of the following features. A method where the deformed osseous anatomy may include a plurality of bones of the patient and where fabricating a 3D physical bone model may include coupling two physical bone models of the 3D physical bone model by way of an interconnect such that the two physical bone models connect in correspondence with patient bones of the deformed osseous anatomy.

A method where the interconnect may include an interface configured to enable a user to reposition one physical bone model in relation to another physical bone model coupled to the interface. A method where the interconnect may include a rigid interconnect. A method where the interconnect may include a detachable interconnect. A method where fabricating a 3D physical bone model may include coupling a plurality of physical bone models of the 3D physical bone model by way of a plurality of interconnects and where the plurality of interconnects may include rigid interconnects and adjustable interconnects.

A method where fabricating a 3D physical bone model may include providing an indicator on a physical bone model of the 3D physical bone model, the indicator configured to convey information about at least one of the osseous anatomy, the patient, and the surgical procedure. A method where the indicator identifies a physical bone model corresponding to a patient bone having a deformed bone condition. A method that includes fabricating a 3D physical instrument of a patient-specific instrument configured for use on the deformed osseous anatomy of the patient for the surgical procedure. A method where the 3D physical instrument may include a bone engagement member configured to engage a bone of the deformed osseous anatomy and to engage a physical bone model of the 3D physical bone model corresponding to the bone of the deformed osseous anatomy. Implementations of the described techniques may include hardware, a method or process, or a computer tangible medium.

In one general aspect, a method may include generating a computer model of osseous anatomy of a patient based on medical imaging data of the osseous anatomy. A method may also include generating a modified computer model of the osseous anatomy, the modified computer model altered from the computer model of the osseous anatomy such that the modified computer model represents the osseous anatomy after a surgical procedure. A method may furthermore include fabricating a preoperative 3D physical bone model of the osseous anatomy based on the computer model of the osseous anatomy before the surgical procedure. A method may in addition include fabricating a postoperative 3D physical bone model of the osseous anatomy after the surgical procedure based on the modified computer model. A method may moreover include. A method may also include providing at least one of the preoperative 3D physical bone model and the postoperative 3D physical bone model to a user. Other embodiments of this aspect include corresponding computer systems, apparatus, and computer programs recorded on one or more computer storage devices, each configured to perform the actions of the methods.

Implementations may include one or more of the following features. A method where generating a modified computer model further may include modifying the computer model based on a prescription provided by a surgeon. A method where generating a modified computer model further may include modifying the computer model in response to user input. A method that includes providing an indicator on one of the preoperative 3D physical bone model and the postoperative 3D physical bone model that distinguishes the preoperative 3D physical bone model from the postoperative 3D physical bone model. A method where the modified computer model is configured to remediate a deformed bone condition of the osseous anatomy. A method that includes fabricating a second postoperative 3D physical bone model based on the computer model of the osseous anatomy. Implementations of the described techniques may include hardware, a method or process, or a computer tangible medium.

In one general aspect, a method may include generating a computer bone model of deformed osseous anatomy of a patient based on medical imaging data of the deformed osseous anatomy. A method may also include fabricating a 3D physical bone model of the deformed osseous anatomy based on the computer bone model. A method may furthermore include generating a computer instrument model of an instrument configured for use in a surgical procedure to remediate the deformed osseous anatomy. A method may in addition include fabricating the instrument based on the computer instrument model. A method may moreover include providing the 3D physical bone model and the instrument to a user. Other embodiments of this aspect include corresponding computer systems, apparatus, and computer programs recorded on one or more computer storage devices, each configured to perform the actions of the methods.

Implementations may include one or more of the following features. A method where the instrument is a patient-specific instrument having a bone engagement member configured to engage a bone of the deformed osseous anatomy and to engage a physical bone model of the 3D physical bone model corresponding to the bone of the deformed osseous anatomy. Implementations of the described techniques may include hardware, a method or process, or a computer tangible medium.

In one general aspect, a kit may include a first 3D physical bone model of deformed osseous anatomy of a patient for an osteotomy procedure. A Kit may also include a preoperative plan for the osteotomy procedure. A Kit may furthermore include a set of patient-specific instruments for one or more stages of the osteotomy procedure. Other embodiments of this aspect include corresponding computer systems, apparatus, and computer programs recorded on one or more computer storage devices, each configured to perform the actions of the methods.

Implementations may include one or more of the following features. A kit may include a second 3D physical bone model having a plurality of physical bone models coupled by way of a plurality of interconnects, the plurality of physical bone models coupled according to a predetermined configuration. A kit may further include where the predetermined configuration is selected from the group having an anatomical position, a normal position, a weight bearing position, one stage of a walking position, one stage of a running position, one stage of a jumping position, one stage of a grasping position, a dorsiflexed position, a plantar flexed position, a laterally rotated position, a medially rotated position, an eversion rotated position, an inversion rotated position, a deformed condition, a corrected condition, a preoperative condition, and a postoperative condition. Implementations of the described techniques may include hardware, a method or process, or a computer tangible medium.

### BRIEF DESCRIPTION OF THE DRAWINGS

The advantages, nature, and additional features of exemplary embodiments of the disclosure will become more fully apparent from the following description and appended claims, taken in conjunction with the accompanying drawings. Understanding that these drawings depict only exemplary embodiments and are, therefore, not to be considered limiting of the disclosure's scope, the exemplary embodiments of the disclosure will be described with additional specificity and detail through use of the accompanying drawings.

FIG. 1A is a flowchart diagram depicting a method for remediating a condition, according to one embodiment.

FIG. 1B is a flowchart diagram depicting a method for remediating a condition, according to one embodiment.

FIG. 2A is a dorsal perspective view of bones of a foot.

FIG. 2B is a lateral perspective view of bones of a foot.

FIG. 2C is a medial perspective view of bones of a foot.

FIG. 2D is a dorsal perspective view of bones of a foot.

FIG. 2E is a view of a foot illustrating common planes of reference for a human foot.

FIG. 3 is a flowchart diagram depicting a method for generating one or more patient-specific instruments configured to address a bone condition, according to one embodiment.

FIG. 4 illustrates an exemplary system configured to generate one or more patient-specific instruments configured to address a bone condition, according to one embodiment.

FIG. 5 illustrates an exemplary apparatus configured to determine a location, patient-specific feature, and/or a trajectory, according to one embodiment.

FIG. 6 illustrates an exemplary provision module configured to provide a preliminary guide model, according to one embodiment.

FIG. 7 illustrates an exemplary design module configured to design a patient-specific guide model, according to one embodiment.

FIG. 8 illustrates an exemplary system configured to generate one or more patient-specific instruments configured to address a bone condition, according to one embodiment.

FIG. 9 illustrates a method for providing a three-dimensional (3D) physical bone model to a customer, according to one embodiment.

FIG. 10 illustrates a method for providing a 3D physical bone model to a customer, according to one embodiment.

FIG. 11 illustrates a method for providing a 3D physical model to a customer, according to one embodiment.

FIG. 12 illustrates a method for providing a 3D physical bone model to a customer, according to one embodiment.

FIG. 13 illustrates an exemplary system, according to one embodiment.

FIG. 14A illustrates an exemplary model, according to one embodiment.

FIG. 14B illustrates an exemplary model, according to one embodiment.

FIG. 15A illustrates an exemplary model, according to one embodiment.

FIG. 15B illustrates exemplary physical instruments, according to one embodiment.

FIG. 16 illustrates exemplary 3D physical bone models, according to one embodiment.

FIG. 17 illustrates an exemplary system, according to one embodiment.

FIG. 18 illustrates a method for providing a 3D physical model for a surgical procedure, according to one embodiment.

FIG. 19 illustrates a method for providing a 3D physical model for a surgical procedure, according to one embodiment.

FIG. 20A illustrates an exemplary user-interface for working with a computer model for a surgical procedure, according to one embodiment.

FIG. 20B illustrates an exemplary user-interface for working with a computer model for a surgical procedure, according to one embodiment.

FIG. 20C illustrates exemplary 3D physical bone models, according to one embodiment.

FIG. 21 illustrates a method for providing a 3D physical model for a surgical procedure, according to one embodiment.

### DETAILED DESCRIPTION

Exemplary embodiments of the disclosure will be best understood by reference to the drawings, wherein like parts are designated by like numerals throughout. It will be readily understood that the components, as generally described and illustrated in the Figures herein, could be arranged and designed in a wide variety of different configurations. Thus, the following more detailed description of the embodiments of the apparatus, system, and method is not intended to limit the scope of the disclosure but is merely representative of exemplary embodiments.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature can pass into the other feature.

As used herein, “coupling”, “coupling member”, or “coupler” refers to a mechanical device, apparatus, member, component, system, assembly, or structure, that is organized, configured, designed, arranged, or engineered to connect, or facilitate the connection of, two or more parts, objects, or structures. In certain embodiments, a coupling can connect adjacent parts or objects at their ends. In certain embodiments, a coupling can be used to connect two shafts together at their ends for the purpose of transmitting power. In other embodiments, a coupling can be used to join two pieces of rotating equipment while permitting some degree of misalignment or end movement or both. In certain embodiments, couplings may not allow disconnection of the two parts, such as shafts during operation. (Search “coupling” on Wikipedia.com Jul. 26, 2021. CC-BY-SA 3.0 Modified. Accessed Jul. 27, 2021.) A coupler may be flexible, semiflexible, pliable, elastic, or rigid. A coupler may join two structures either directly by connecting directly to one structure and/or directly to the other or indirectly by connecting indirectly (by way of one or more intermediary structures) to one structure, to the other structure, or to both structures.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Standard medical planes of reference and descriptive terminology are employed in this disclosure. While these terms are commonly used to refer to the human body, certain terms are applicable to physical objects in general. A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body from the side which has a particular condition or structure. Proximal means toward the trunk of the body. Proximal may also mean toward a user, viewer, or operator. Distal means away from the trunk. Distal may also mean away from a user, viewer, or operator. Dorsal means toward the top of the foot or other body structure. Plantar means toward the sole of the foot or toward the bottom of the body structure.

Antegrade means forward moving from a proximal location/position to a distal location/position or moving in a forward direction. Retrograde means backward moving from a distal location/position to a proximal location/position or moving in a backwards direction. Sagittal refers to a midline of a patient's anatomy, which divides the body into left or right halves. The sagittal plane may be in the center of the body, splitting it into two halves. Prone means a body of a person lying face down. Supine means a body of a person lying face up.

“Patient-specific guide” refers to a guide designed, engineered, and/or fabricated for use with a specific patient. In one aspect, a patient-specific guide is unique to a patient and may include features unique to the patient such as a surface contour or other features.

“Patient-specific cutting guide” refers to a cutting guide designed, engineered, and/or fabricated for use with a specific patient. In one aspect, a patient-specific cutting guide is unique to a patient and may include features unique to the patient such as a surface contour or other features.

“Patient-specific resection guide” refers to a guide designed, engineered, and/or fabricated for use in resection for a specific patient. In one aspect, a patient-specific resection guide is unique to a patient and may include features unique to the patient such as a surface contour or other features.

“Patient-specific instrument” (PSI) refers to a structure, device, guide, tool, instrument, apparatus, member, component, system, assembly, module, or subsystem that is adjusted, tailored, modified, organized, configured, designed, arranged, engineered, and/or fabricated to specifically address the anatomy, physiology, condition, abnormalities, needs, or desires of a particular patient. In certain aspects, one patient. In one aspect, a patient specific instrument is unique to a single patient and may include features unique to the patient such as a surface contour, component position, component orientation, and/or other features. In other aspects, one patient specific instrument may be useable with a number of patients having a particular class of characteristics.

“Instrument” refers to any apparatus, device, or object that can be used by a user. An instrument may be used for a specific or a generic purpose. An instrument may also be referred to as instrumentation. Instrumentation may refer to a single instrument and/or a plurality of instruments. An instrument may be specifically designed, constructed or fabricated for use by a specific user, for a single use, and/or for a plurality of uses. A patient-specific instrument is one example of an instrument.

“Positioner” or “positioning guide” refers to any structure, apparatus, surface, device, system, feature, or aspect configured to position, move, translate, manipulate, or arrange one object in relation to another. In certain embodiments, a positioner can be used for one step in surgical procedure to position, arrange, orient, and/or reduce one bone or bone fragment relative to another. In such embodiments, the positioner may be referred to as a bone positioner. In certain embodiments, the term positioner or positioning guide may be preceded by an adjective that identifies the structure, implement, component, or instrument that may be used with, positioned by, and/or guided by with the positioner. For example, a “pin positioner” may be configured to accept a pin or wire such as a K-wire and serve to position or place the pin relative to another structure such as a bone.

As used herein, “preoperative plan” refers to a plan for performing a surgical procedure. Depending on the complexity of a surgery, a preoperative plan can be very simple and generic or very detailed and specific to a particular surgical procedure. In one aspect, a preoperative plan may include very detailed and specific step by step instructions for the surgical procedure. The instructions may be ordered according to a specific order for accomplishing a desired outcome. In certain embodiments, a preoperative plan may indicate which instruments, machines, systems, test, and/or personnel to use for the surgical procedure. A preoperative plan can take many forms and formats based on the needs and desires of the users of the preoperative plan. In one embodiment, the preoperative plan is a report that is displayed on a screen or that can be printed onto paper. In another embodiment, the preoperative plan may include instructions for operation planning software. In another embodiment, the preoperative plan may include instructions for surgical rehearsal tools, including software. In another embodiment, the preoperative plan may include instructions for operation planning using virtual reality or augmented reality software.

“Compressor” refers to any apparatus, device, or system that can function as an active compression instrument. A compressor functions to bring two objects closer to or in contact with each other.

“Post” refers to any apparatus, structure, device, system, and/or component that extends from another structure. In certain embodiments, a post can be cylindrical.

“Anatomical position” refers to a standard, accepted, reference position used in anatomy and medicine to describe the relative positioning of body structures and anatomical terms. (© ChatGPT 3.5 Version, Modified, accessed chat.openai.com/chat Feb. 9, 2024).

“Cut surface” or “cut face” refers to a surface of an object that is created or formed by the removal of one or more parts of the object that includes the original surface. Cut surfaces or cut faces can be created using a variety of methods, tools, or apparatuses and may be formed using a variety of removal actions, including, but not limited to, fenestrating, drilling, abrading, cutting, sawing, chiseling, digging, scrapping, and the like. Tools and/or methods used for forming a cut surface or cut face can include manual, mechanical, motorized, hydraulic, automated, robotic, and the like. In certain embodiments, the cut surface(s) are planar.

“Reference” refers to any apparatus, structure, device, system, component, marking, and/or indicator organized, configured, designed, engineered, and/or arranged to serve as a source of information or a point of comparison used to support or establish knowledge, truth, or quality. (© ChatGPT January 9 Version, Modified, accessed chat.openai.com/chat Jan. 28, 2023). In certain embodiments, a reference can serve as a starting point or initial position for one or more steps in a surgical procedure. In certain embodiments, “reference” can be combined with an adjective describing the reference. For example, a “model reference” is a reference within a model such as a computer model. A model reference refers to any feature, aspect, and/or component within a model. Examples of a model reference include, but are not limited to, a point, a plane, a line, a plurality of points, a surface, an anatomical structure, a shape, or the like. An “anatomical reference” is a reference within, on, near, or otherwise associated with an anatomical structure such as a bone. A reference (e.g., model, actual, virtual, and/or real) may also be referred to as a reference feature.

“Reference feature” refers to a feature configured for use as a point, plane, axis, or line of reference (aka a reference). A reference can be used to position, measure, orient, fixation, couple, engage, and/or align one object or structure with another object or structure. In certain embodiments, a reference or reference feature can serve as a baseline, a ground truth, a waypoint, a control point, a landmark, and/or the like. A reference feature can facilitate moving from one coordinate system to a position or orientation on or in an actual object, structure, device, apparatus, anatomical structure, or the like. Advantageously, a reference feature can coordinate objects, models, or structures in a digital or virtual model or representation with corresponding objects or structures (e.g., anatomical structures) of actual physical objects or structures. Said another way, a reference feature can serve to map from a virtual or modeled object to an actual or physical object.

“Reference feature guide” refers to a guide that serves to aid in forming and/or deploying one or more reference features. Examples of reference feature guides include but are not limited to a hole, a round hole, a channel, a slot, a plurality of holes, a fence, a backstop, a guard, a fastener, a pilot hole, a blind hole, a chute, a ramp, or the like.

“Anatomical structure” refers to any part or portion of a part of a body of a person, animal, or other patient. Examples of anatomical structures, include but are not limited to, a bone, bones, soft tissue, a joint, joints, skin, hard tissue, teeth, mouth, eyes, hair, nails, fingers, toes, legs, arms, torso, vertebrae, ligaments, tendons, organs, or the like.

“Anatomical reference” or “anatomical landmark” refers to any reference or landmark that is, or is on, or is in, or is otherwise associated, with an anatomical structure. Examples of anatomical structures, include but are not limited to, a bone, bones, soft tissue, a joint, joints, skin, hard tissue, teeth, mouth, eyes, hair, nails, fingers, toes, legs, arms, torso, vertebrae, ligaments, tendons, organs, a hole, a post, a plurality of holes, a plurality of posts, or the like.

“Deformity” refers to an abnormality or deviation from the normal shape, structure, orientation, trajectory, or function of a body part. This can be due to congenital conditions, injuries, diseases, or other factors that alter the normal development or functioning of a part of the body. (© ChatGPT January 30 Version, accessed chat.openai.com/chat Feb. 7, 2023).

“Configuration” refers to an arrangement, setup, or values of one or more parts, features, settings, components, aspects, structures, or the like as a module, component, apparatus, device, system, framework, platform, dashboard, assembly, or the like. Examples of configurations can include how dials are setup on a dashboard, levers are set on a control board, switches are set within a controller, bones are arranged within a hand, foot, or limb, or the like.

“Interconnect” refers to a structure configured to join at least two other structures. In one embodiment, the interconnect may be a mechanical structure that may physically connect one structure to another structure. In other embodiments, an interconnect may be embodied as a fastener that enables permanent or temporary joining of one structure to another structure. In still other embodiments, an interconnect may be embodied as a joint or hinge configured to enable one or both structured joined by the interconnect to move relative to each other while remaining joined. In one embodiment, the interconnect may be configured to convey fluid and/or an electric signal between the at least two other structures. For example, the interconnect may comprise a channel or tube configured to convey air between a first opening and a second opening in the channel or tube. Examples of an interconnect include, but are not limited to, a pipe, a tunnel, a chamber, a channel, or the like. Other examples of an interconnect include, but are not limited to, solid material that can be additively manufactured between two structures, a snap, a hook and loop system, a spring, a tether, or the like.

As used herein, an “interface,” “user interface,” or “engagement interface” refers to an area, a boundary, structure, or a place at which two separate and/or independent structures, members, apparatus, assemblies, components, and/or systems join, connect, are coupled, or meet and act on, or communicate, mechanically and/or electronically, with each other. In certain embodiments, “interface” may refer to a surface forming a common boundary of two bodies, spaces, structures, members, apparatus, assemblies, components, or phases. (search “interface” on Merriam-Webster.com. Merriam-Webster, 2021. Web. 15 Nov. 2021. Modified.) In certain embodiments, the term interface may be used with an adjective that identifies a type or function for the interface. For example, an engagement or coupling interface may refer to one or more structures that interact, connect, or couple to mechanically join or connect two separate structures, each connected to a side of the interface. In another example, a user interface may refer to one or more mechanical, electrical, or electromechanical structures that interact with or enable a user to provide user input, instructions, input signals, data, or data values and receive output, output data, or feedback.

As used herein, a “handle” refers to a structure used to hold, control, or manipulate a device, apparatus, component, tool, or the like. A “handle” may be designed to be grasped and/or held using one or two hands of a user.

“Rigid interconnect” refers to an interconnect that is rigid such that two structures connected by the interconnect remain in a single position and/or orientation and cannot be repositioned without breaking or decoupling or disconnecting the interconnect.

“Detachable interconnect” refers to an interconnect that is configured to permit two structures connected by the interconnect to be decoupled without damaging the interconnect. In certain embodiments, a detachable interconnect may connect two structures such that the structures remain in a single position and/or orientation until the two structures a detached by decoupling the interconnect.

“Adjustable interconnect” refers to an interconnect that connects or coupled a first structure to a second structure a single position and/or orientation relative to each other. Furthermore, an adjustable interconnect includes a device, component, mechanism, and/or system configured to permit adjustment of a single position and/or orientation of at least one of the first structure in relation to the second structure. In certain embodiments, an adjustable interconnect can also maintain a new position and/or orientation of the first structure relative to the second structure after an adjustment to the position and/or orientation of at least one of the first structure and the second structure.

As used herein, an “indicator” refers to an apparatus, device, component, system, assembly, mechanism, hardware, software, firmware, circuit, module, set of data, text, number, code, symbol, a mark, marker, or logic structured, organized, configured, programmed, designed, arranged, or engineered to convey information or indicate a state, condition, mode, context, location, or position to another apparatus, device, component, system, assembly, mechanism, hardware, software, firmware, circuit, module, and/or a user of an apparatus, device, component, system, assembly, mechanism, hardware, software, firmware, circuit, module that includes, or is associated with the indicator. The indicator can include one or more of an audible signal, a token, a presence of a signal, an absence of a signal, a tactile signal, a visual signal or indication, a visual marker, a visual icon, a visual symbol, a visual code, a visual mark, and/or the like. In certain embodiments, “indicator” can be used with an adjective describing the indicator. For example, a “mode indicator” is an indicator that identifies or indicates a mode.

As used herein, a “marking” or “marker” refers to a symbol, letter, lettering, word, phrase, icon, design, color, diagram, indicator, figure, structure, device, apparatus, surface, component, system, or combination of these designed, intended, structured, organized, configured, programmed, arranged, or engineered to communication information and/or a message to a user receiving, viewing, or encountering the marking. The marking or “marker” can include one or more of a tactile signal, a visual signal or indication, an audible signal, and the like. In one embodiment, a marking may comprise a number or set letters, symbols, or words positioned on a surface, structure, color, color scheme, or device to convey a desired message or set of information.

As used herein, a “graft,” “tissue graft,” and/or “bone graft” refers to a surgical procedure to move tissue (hard and/or soft tissue) from one site to another on the body, or from another creature, without bringing its own blood supply with the tissue. Instead, a new blood supply grows in after the tissue is placed. A similar technique where tissue is transferred with the blood supply intact is called a flap. (Search ‘Graft (surgery)’ on Wikipedia.com Apr. 21, 2021. Modified. Accessed Aug. 30, 2021.) “Graft” may also be used to refer to the tissue and/or synthetic composition used for a graft surgical procedure. Bone grafting is a surgical procedure that replaces missing bone in order to repair bone fractures. Bone generally has the ability to regenerate completely but may require a small fracture space and/or a scaffold to do so. Bone grafts may be autologous (bone harvested from the patient's own body, often from the iliac crest), allograft (cadaveric bone usually obtained from a bone bank), or synthetic (often made of hydroxyapatite (HA) or other naturally occurring and biocompatible substances) with similar mechanical properties to bone. Generally, bone grafts are expected to be reabsorbed and replaced as natural bone heals over a few months' time. (Search ‘Bone Grafting’ on Wikipedia.com Apr. 21, 2021. Modified. Accessed Aug. 30, 2021.) Certain grafts may include a combination of autograft, isograft, allograft, xenograft, and/or synthetic materials in a single bone graft composition. An example of such a compositions, include but is not limited to, Demineralized bone matrix (DBM). Bone graft compositions may include bone morphogenetic proteins (BMPs).

As used herein, a “condition” refers to a state of something with regard to its appearance, quality, or working order. In certain aspects, a condition may refer to a patient's state of health or physical fitness or the state of health or physical fitness of an organ or anatomical part of a patient. In certain embodiments, a condition may refer to an illness, defect, disease, or deformity of a patient or of an organ or anatomical part of a patient. (Search “condition” on wordhippo.com. WordHippo, 2021. Web. Accessed 8 Dec. 2021. Modified.)

“Bone condition” refers to any of a variety of conditions of bones of a patient. Generally, a bone condition refers to an orientation, position, and/or alignment of one or more bones of the patient relative to other anatomical structures of the body of the patient. Bone conditions may be caused by or result from deformities, misalignment, malrotation, fractures, joint failure, and/or the like. A bone condition includes, but is not limited to, any angular deformities of one or more bone segments in either the lower or upper extremities (for example, tibial deformities, calcaneal deformities, femoral deformities, and radial deformities). Alternatively, or in addition, “bone condition” can refer to the structural makeup and configuration of one or more bones of a patient. Thus bone condition may refer to a state or condition of regions, a thickness of a cortex, bone density, a thickness and/or porosity of internal regions (e.g. whether it is calcaneus or solid) of the bone or parts of the bone such as a head, a base, a shaft, a protuberance, a process, a lamina, a foramen, and the like of a bone, along the metaphyseal region, epiphysis region, and/or a diaphyseal region.

“Deformed bone condition” refers to any of a variety of conditions of bones of a patient are different from normal, standard or accepted bone conditions. Generally, a deformed bone condition refers to an orientation, position, and/or alignment of one or more bones of the patient relative to other anatomical structures of the body of the patient that is atypical, abnormal, and/or different from average characteristics. Deformed bone conditions may be caused by or result from deformities, misalignment, malrotation, fractures, joint failure, and/or the like. A deformed bone condition includes, but is not limited to, any angular deformities of one or more bone segments in either the lower or upper extremities (for example, tibial deformities, calcaneal deformities, femoral deformities, and radial deformities).

“Data” refers to a set of information organized in a way that facilitates communication of the information to a receiver. The receiver may be a person or animal or an electronic component, circuit, assembly, or the like. Data can be represented as signal or values represented in any numbering and/or alphabet system. Data can be stored in one representation in an analog or digital format and conveyed to a receiver in another format suitable for the receiver to interpret and understand the data. Data can include both data that stores specific information as well as metadata which is data that describes the data that stores the specific information. Data can be organized in a structured or unstructured format. “Structured data” refers to data within a data structure that is organized according to a predefined format, protocol, or configuration such that the structure may be used to facilitate working with the data. Examples of structured data include, but are not limited to, files, databases, database records, database tables, database schemas, serialized objects, directories, and the like. “Unstructured data” refers to data stored without a particular organization, predefined format, protocol, or configuration. Examples of unstructured data include, but are not limited to, content of a text message, content of an email message, text content of a file, content of a document, and the like. Often the term “data” will be used in connection with one or more adjectives that identify a type or purpose for the data, examples include “user data”, “input data”, “output data”, “sensor data”, “patient data”, “system data”, “map data”, and the like. “Sensor data” refers to any data or information registered by one or more sensors. Examples of sensor data include an amount of current passing through the sensor, an amount of voltage across the sensor, an amount of electrical resistance through the sensor, an amount of strain experienced by the sensor, an acceleration vector, a deceleration vector, an orientation, an orientation angle, a direction, and the like.

As used herein, “implant” refers to a medical device manufactured to replace a missing biological structure, support a damaged biological structure, or enhance an existing biological structure. Often medical implants are man-made devices, but implants can also be natural occurring structures. The surface of implants that contact the body may be made of, or include a biomedical material such as titanium, cobalt chrome, stainless steel, carbon fiber, another metallic alloy, silicone, polymer, Synthetic polyvinyl alcohol (PVA) hydrogels, biomaterials, biocompatible polymers such as PolyEther Ether Ketone (PEEK) or a polylactide polymer (e.g. PLLA) and/or others, or apatite, or any combination of these depending on what is functional and/or economical. Implants can have a variety of configurations and can be wholly, partially, and/or include a number of components that are flexible, semiflexible, pliable, elastic, supple, semi-rigid, or rigid. In some cases, implants contain electronics, e.g. artificial pacemaker and cochlear implants. Some implants are bioactive, such as subcutaneous drug delivery devices in the form of implantable pills or drug-eluting stents. Orthopedic implants may be used to alleviate issues with bones and/or joints of a patient's body. Orthopedic implants can be used to treat bone fractures, osteoarthritis, scoliosis, spinal stenosis, discomfort, and pain. Examples of orthopedic implants include, but are not limited to, a wide variety of pins, rods, screws, anchors, spacers, sutures, all-suture implants, ball all-suture implants, self-locking suture implants, cross-threaded suture implants, plates used to anchor fractured bones while the bones heal or fuse together, and the like. (Search “implant (medicine)” on Wikipedia.com May 26, 2021. CC-BY-SA 3.0 Modified. Accessed Jun. 30, 2021.)

As used herein, a “body” refers to a main or central part of a structure. The body may serve as a structural component to connect, interconnect, surround, enclose, and/or protect one or more other structural components. A body may be made from a variety of materials including, but not limited to, metal, plastic, ceramic, wood, fiberglass, acrylic, carbon, biocompatible materials, biodegradable materials or the like. A body may be formed of any biocompatible materials, including but not limited to biocompatible metals such as Titanium, Titanium alloys, stainless steel alloys, cobalt-chromium steel alloys, nickel-titanium alloys, shape memory alloys such as Nitinol, biocompatible ceramics, and biocompatible polymers such as Polyether ether ketone (PEEK), a polylactide polymer (e.g. PLLA), nylon 12, and/or others. In one embodiment, a body may include a housing or frame, or framework for a larger system, component, structure, or device. A body may include a modifier that identifies a particular function, location, orientation, operation, and/or a particular structure relating to the body. Examples of such modifiers applied to a body, include, but are not limited to, “inferior body,” “superior body,” “lateral body,” “medial body,” and the like.

As used herein, “side” refers to a structure or part of a structure including, but not limited to: one of a longer bounding surfaces or lines of an object especially contrasted with the ends, a line or surface forming a border or face of an object, either surface of a thin object, a bounding line or structure of a geometric figure or shape, and the like. (search “side” on Merriam-Webster.com. Merriam-Webster, 2021. Web. 3 Aug. 2021. Modified.) A side can also refer to a geometric edge of a polygon (two-dimensional shape) and/or a face or surface of a polyhedron (three-dimensional shape). (Search “side” on Wikipedia.com Jul. 21, 2021. CC-BY-SA 3.0 Modified. Accessed Aug. 3, 2021.) Side can also refer to a location on a structure. For example, a side can be a location on a structure at, or near, a furthest position away from a central axis of the structure. As used herein, the term “side” can include one or more modifiers that define and/or orient and/or distinguish the side of an object from others based on based on where and/or how the object is deployed within or in relation to a second object. For example, in the context of an implant for a patient, sides of the implant may be labeled based on where the sides are relative to the patient when the implant is deployed. As one example, an “anterior side” of an implant, instrument, anatomical structure, or other structure refers to a side that is anterior to other sides of the structure in relation to a patient when the structure is deployed in the patient. As another example, in the context of an instrument used with a patient, sides of the instrument may be labeled based on where the sides are when the instrument is being used for its purpose. As one example, a “front side” of an instrument refers to a side that is facing a user of the instrument when the instrument is in use.

As used herein, “bone engagement surface” refers to a surface of an object, instrument, or apparatus, such as an implant that is oriented toward or faces one or more bones of a patient. In one aspect, the bone engagement surface may abut, touch, or contact a surface of a bone. In another aspect, the bone engagement surface or parts of the bone engagement surface may be close to, but not abut, touch, or contact a surface of the bone. In certain aspects, the bone engagement surface can be configured to engage with a surface of one or more bones. Such a bone engagement surface may include projections and recesses that correspond to and match projections and recesses of the one or more bone surfaces.

“Bone engagement member” refers to an apparatus, instrument, structure, device, component, member, system, assembly or module structured, organized, configured, designed, arranged, or engineered to connect, join, link, contact, touch, abut, interface with, couple to, or engage with a bone, a bone part, bony topography (e.g., bone spurs and calcifications), anatomical bone feature, and/or a bone fragment. The connection, coupling, linkage, contact, or engagement may be a mechanical connection or interconnection. A bone engagement member may enable temporary engagement with a bone or bone fragment or permanent engagement with a bone or bone fragment. A bone engagement member may include a bone engagement surface, a bone engagement feature, a body section that supports the bone engagement surface, or the like. In certain embodiments, a bone engagement member may include a bone probe or a joint seeker. In one embodiment, a bone engagement member may include a landmark registration feature. Alternatively, or in addition, a bone engagement member can include a bone attachment feature configured to engage with bone and/or to cooperate with a fastener to engage with bone. A patient-specific bone engagement member is a bone engagement member that includes one or more aspects that are patient-specific. The patient-specific aspects can include, but are not limited to, a surface contour, a contour for a part of a surface, a position for a resection feature, a size, shape and/or configuration of a resection feature, a position, size, shape, and/or number of bone attachment features, or the like.

As used herein, “bone-facing side” refers to a side of an object, structure, instrument, or apparatus, such as an implant or instrument that is oriented toward or faces one or more bones of a patient when a device that includes the bone-facing side is in use. In one aspect, the bone-facing side may abut, touch, or contact a surface of a bone. In another aspect, the bone-facing side or parts of the bone-facing side may be close to, but not abut, touch, or contact a surface of the bone.

“Non-bone-facing side” refers to a side of an object, structure, instrument, or apparatus, such as an implant or instrument that is not oriented toward and/or does not face one or more bones of a patient during use of a device that includes the non-bone-facing side. In certain embodiments, a non-bone-facing side can be a side that is directly opposite a bone-facing side of the same device, object, structure, or apparatus.

“Cortical surface” refers to a surface of cortical bone. “Cortical bone” refers to a type of bone tissue. Cortical bone is a type of bone tissue typically found between an external surface of a bone and an interior area of the bone. Cortical bone is more dense and typically stronger structurally than other types of bone tissue.

“Predetermined Position” refers to a position that is decided, determined, finalized, and/or defined earlier in time. In certain embodiments, a predetermined position is a desired, designed, and/or engineered position of a first object in relation to a second object. Thus, a predetermined position is a planned position for the two objects in relation to each other. In certain embodiments, one or both of the two objects may be moved relative to each other to accomplish the predetermined position and the predetermined position may become the final position. In other embodiments, the two objects may be moved towards the predetermined position but may not reach the exact predetermined position due to some impediment and/or interference or a decision to change the predetermined position to a new position. In certain aspects, a predetermined position may be a position that is decided after a process of recommendation, review, and/or analysis, and final approval such that a position may not become a predetermined position until the process is completed. For example, in a medical patient-specific instrument or technique design process a position may not become the predetermined position until a surgeon or other doctor provides final approval for the position. In certain embodiments, a predetermined position may be indicated, designated, illustrated, defined, and/or explained in a preoperative plan.

As used herein, a “deploy” or “deployment” refers to an act, action, process, system, method, means, or apparatus for inserting an implant or prosthesis into a part, body part, and/or patient. “Deploy” or “deployment” can also refer to an act, action, process, system, method, means, or apparatus for placing something into therapeutic use. A device, system, component, medication, drug, compound, or nutrient may be deployed by a human operator, a mechanical device, an automated system, a computer system or program, a robotic system, or the like.

“Joint” or “Articulation” refers to the connection made between bones in a human or animal body which link the skeletal system to form a functional whole. Joints may be biomechanically classified as a simple joint, a compound joint, or a complex joint. Joints may be classified anatomically into groups such as joints of hand, elbow joints, wrist joints, axillary joints, sternoclavicular joints, vertebral articulations, temporomandibular joints, sacroiliac joints, hip joints, knee joints, articulations of foot, and the like. (Search “joint” on Wikipedia.com Dec. 19, 2021. CC-BY-SA 3.0 Modified. Accessed Jan. 20, 2022.)

“Contour” refers to an outline representing or bounding a shape or form of an object. Contour can also refer to an outside limit of an object, area, or surface of the object. (Search “contour” on wordhippo.com. WordHippo, 2023. Web. Modified. Accessed 13 Jun. 2023.)

“Topographical” refers to the physical distribution of parts, structures, or features on the surface of, or within, an organ or other anatomical structure, or organism. (Search “define topographical” on google.com. Oxford Languages, Copyright 2022. Oxford University Press. Web, Modified. Accessed 15 Feb. 2022.)

“Landmark registration feature” refers to a structure configured to engage, contact, or abut a feature, aspect, attribute, or characteristic of a first object to orient and/or position a second object that includes the landmark registration feature with respect to the first object. A variety of structures can serve as a landmark registration feature. For example, a surface, a probe, a finger, a wing, an arm, an opening, or the like can function as landmark registration features. A landmark registration feature can be of a variety of shapes and thus can include a protrusion, a projection, a tuberosity, a cavity, a void, a divot, a tab, an extension, a hook, a curve, or the like.

“Landmark” refers to a structure on, in, or around a structure that can be used to serve as a reference for positioning, orienting, translating, rotating, or otherwise manipulating a second object or structure. For example, a landmark may include a protrusion, a projection, a tuberosity, a cavity, a void, a divot, a tab, an extension, a hook, a curve, or the like. In the context of bones of a patient, a landmark can include any protuberance, eminence, bony topography, anatomical features, calcifications, void, divot, concave section, sesamoid, bone spur or other feature on, or extending from, a bone of a patient. A landmark refers to any structure of an anatomical structure that is referenced, contacted, engaged with and/or associated with a landmark registration feature. In certain embodiments, a landmark is unique to one patient.

“Bone attachment feature” refers to a structure, feature, component, aspect configured to securely connect, couple, attach, and/or engage a structure, component, object, or body with a bone and/or a bone fragment. Examples of a bone attachment feature, include, but are not limited to, a pin, K-wire, screw, or other fastener alone, or in combination with, a hole, passage, and/or opening.

“Position indicator” refers to any apparatus, structure, device, system, and/or component organized, configured, designed, engineered, and/or arranged to serve as an indicator of a position for one or more things, objects, structures, apparatuses, systems, features, aspects, attributes or the like. Examples of a position indicator include, but are not limited to, a crosshair, cross hairs, a pin, a wire, a fastener, a hole, an opening, a post, a prong, a probe, a needle, an arrow, a marking, or the like. In certain embodiments, an indicator may communicate a position of one structure or component or system in relation to another. A position indicator may indicate a position of one object relative to another, may indicate a relationship between two objects, may indicate a trajectory of one object relative to another, or the like.

As used herein, a “stop” refers to an apparatus, instrument, structure, member, device, component, system, or assembly structured, organized, configured, designed, arranged, or engineered to prevent, limit, impede, stop, or restrict motion or movement and/or operation of the another object, member, structure, component, part, apparatus, system, or assembly.

“Window” refers to an opening and/or a plurality of openings in a body, side, wall, side door, roof, vehicle, system, component, or other structure that allows the passage of electromagnetic radiation including radio ways, x-rays, visible light, light, and the like. A window may also permit passage of sound, gases, fluids, liquids, or other elements. (Search “window” on Wikipedia.com Aug. 31, 2022. Modified. Accessed Sep. 21, 2022.). A window can be opaque, semi-opaque, translucent, radiolucent, or transparent. A window can include a single opening having a single geometric shape or a plurality of openings each of a single geometric shape or combination of a variety of geometric shapes. In certain embodiments, a window may be referred to as a radiolucent window. A radiolucent window may permit passage of some or all radio ways through the window.

“Radiolucent window” refers to a window that permits the passage of radiant energy and electromagnetic radiant energy, in particular, such as x-rays used in an x-ray machine and/or in a fluoroscopy imaging device.

As used herein, a “fastener”, “fixation device”, or “fastener system” refers to any structure configured, designed, or engineered to join two structures. Fasteners may be made of a variety of materials including metal, plastic, composite materials, metal alloys, plastic composites, and the like. Examples of fasteners include, but are not limited to screws, rivets, bolts, nails, snaps, hook and loop, set screws, bone screws, nuts, posts, pins, thumb screws, and the like. Other examples of fasteners include, but are not limited to wires, Kirschner wires (K-wire), anchors, bone anchors, plates, bone plates, intramedullary nails or rods or pins, implants, sutures, soft sutures, soft anchors, tethers, interbody cages, fusion cages, and the like.

In certain embodiments, the term fastener may refer to a fastener system that includes two or more structures configured to combine to serve as a fastener. An example of a fastener system is a rod or shaft having external threads and an opening or bore within another structure having corresponding internal threads configured to engage the external threads of the rod or shaft.

In certain embodiments, the term fastener may be used with an adjective that identifies an object or structure that the fastener may be particularly configured, designed, or engineered to engage, connect to, join, contact, or couple together with one or more other structures of the same or different types. For example, a “bone fastener” may refer to an apparatus for joining or connecting one or more bones, one or more bone portions, soft tissue and a bone or bone portion, hard tissue and a bone or bone portion, an apparatus and a bone or portion of bone, or the like.

In certain embodiments, a fastener may be a temporary fastener. A temporary fastener is configured to engage and serve a fastening function for a relatively short period of time. Typically, a temporary fastener is configured to be used until another procedure or operation is completed and/or until a particular event. In certain embodiments, a user may remove or disengage a temporary fastener. Alternatively, or in addition, another structure, event, or machine may cause the temporary fastener to become disengaged.

As used herein, a “fixator” refers to an apparatus, instrument, structure, device, component, member, system, assembly, or module structured, organized, configured, designed, arranged, or engineered to connect two bones or bone fragments or a single bone or bone fragment and another fixator to position and retain the bone or bone fragments in a desired position and/or orientation. Examples of fixators include both those for external fixation as well as those for internal fixation and include, but are not limited to pins, wires, Kirschner wires, screws, anchors, bone anchors, plates, bone plates, intramedullary nails or rods or pins, implants, interbody cages, fusion cages, and the like.

As used herein, a “fixation” or “fixation device” refers to an apparatus, instrument, structure, device, component, member, system, assembly, step, process, or module structured, organized, configured, designed, arranged, or engineered to connect two structures either permanently or temporarily. The two structures may be one or the other or both of manmade and/or biological tissues, hard tissues such as bones, teeth or the like, soft tissues such as ligament, cartilage, tendon, or the like. In certain embodiments, fixation is used as an adjective to describe a device or component or step in securing two structures such that the structures remain connected to each other in a desired position and/or orientation. Fixation devices can also serve to maintain a desired level of tension, compression, or redistribute load and stresses experienced by the two structures and can serve to reduce relative motion of one part relative to others. Examples of fixation devices are many and include both those for external fixation as well as those for internal fixation and include, but are not limited to pins, wires, Kirschner wires (K-wires), screws, anchors, bone anchors, plates, bone plates, intramedullary nails or rods or pins, implants, interbody cages, fusion cages, and the like.

As used herein, an “anchor” refers to an apparatus, instrument, structure, member, part, device, component, system, or assembly structured, organized, configured, designed, arranged, or engineered to secure, retain, stop, and/or hold, an object to or at a fixed point, position, or location. An anchor may be coupled and/or connected to a flexible member such as a tether, chain, rope, wire, thread, suture, suture tape, or other like object. Alternatively, or in addition, an anchor may also be coupled, connected, and/or joined to a rigid object or structure. In certain embodiments, an anchor can be a fixation device. Said another way, a fixation device can function as an anchor. For example, an anchor pin is a pin, fastener, or K-wire that cooperates with a rigid structure to provide an anchor.

“Connector” refers to any structure configured, engineered, designed, adapted, and/or arranged to connect one structure, component, element, or apparatus to another structure, component, element, or apparatus. A connector can be rigid, pliable, elastic, flexible, and/or semiflexible. Examples of a connector include but are not limited to any fastener.

As used herein, a “sleeve” refers to structure that is narrow and longer longitudinally than the structure is wide. In certain embodiments, a sleeve serves to surround, enclose, wrap, and/or contain something else. In certain embodiments, a sleeve may surround, enclose, wrap, and/or contain a passage or void. (Search “sleeve” on wordhippo.com. WordHippo, 2021. Web. Accessed 15 Nov. 2021. Modified.) In certain embodiments, the term sleeve may be preceded by an adjective that identifies the structure, implement, component or instrument that may be used with, inserted into or associated with the sleeve. For example, a “pin sleeve” may be configured to accept a pin or wire such as a K-wire, a “drive sleeve” may be configured to accept a drill or drill bit, a “fixation member sleeve” may be configured to accept a fastener or fixation member.

As used herein, a “long bone” refers to a bone of a patient having a length greater than a width of the bone. Long bone is one of five types of bones: long, short, flat, irregular and sesamoid. Long bones, especially the femur and tibia, can be subjected to most of the load during daily activities. Long bones grow primarily by elongation of the diaphysis, with an epiphysis at each end of the growing bone. The ends of epiphyses are covered with hyaline cartilage (“articular cartilage”). The longitudinal growth of long bones is a result of endochondral ossification at the epiphyseal plate. The long bone category type includes the femur, tibia, and fibula of the legs; the humerus, radius, and ulna of the arms; metacarpals and metatarsals of the hands and feet, the phalanges of the fingers and toes, and the clavicles or collar bones in humans or other patients. The outside of the long bone consists of a layer of connective tissue called the periosteum. Additionally, the outer shell of the long bone is compact bone, then a deeper layer of cancellous bone (spongy bone) which includes a medullary cavity that includes bone marrow. (Search “long bone” on Wikipedia.com May 14, 2021. CC-BY-SA 3.0 Modified. Accessed Jul. 26, 2021.)

“Talar dome” refers to part of a talus bone. Specifically, the talar dome refers to the superior convex surface and/or area of the talus. The talar dome may also be referred to as a trochlea of the talus. The talar dome is part of the talus body.

“Bone fragment” or “fragment” refers to a part of a bone that is normally part of another bone of a patient. A bone fragment may be separate from another bone of a patient due to a deformity or trauma. In one aspect, the bone the bone fragment is normally connected or joined with is referred to as a parent bone.

As used herein, “manufacturing tool” or “fabrication tool” refers to a manufacturing or fabrication process, tool, system, or apparatus which creates an object, device, apparatus, feature, or component using one or more source materials. A manufacturing tool or fabrication tool can use a variety of manufacturing processes, including but not limited to additive manufacturing, subtractive manufacturing, forging, casting, and the like. The manufacturing tool can use a variety of materials including polymers, thermoplastics, metals, biocompatible materials, biodegradable materials, ceramics, biochemicals, and the like. A manufacturing tool may be operated manually by an operator, automatically using a computer numerical controller (CNC), or a combination of these techniques.

As used herein, “osteotomy procedure” or “surgical osteotomy” refers to a surgical operation in which one or more bones are cut to shorten or lengthen them or to change their alignment. The procedure can include removing one or more portions of bone and/or adding one or more portions of bone or bone substitutes. (Search “osteotomy” on Wikipedia.com Feb. 3, 22, 2021. CC-BY-SA 3.0 Modified. Accessed Feb. 15, 2022.)

“Deformed osseous anatomy” refers to one or more anatomical structures that include or are one or more bones of a patient. The deformed osseous anatomy includes at least one bone that is a deformed bone condition. A deformed osseous anatomy can include a single bone or a collection of bones for a particular anatomical structure including but not limited to a foot, an ankle, a leg, a knee, a hip, a spine, a hand, an elbow, an arm, a shoulder, a neck, and a skull. Deformed osseous anatomy can further include abnormalities or deviations from the typical or expected structure, shape, size, or alignment of bones within the body. Deformed osseous anatomy involves variations or distortions in bone morphology that may result from congenital conditions, genetic disorders, developmental anomalies, trauma, disease processes, and/or other factors. (© ChatGPT 3.5 Version, Modified, accessed chat.openai.com/chat Feb. 9, 2024).

“Osseous anatomy” refers to one or more bones positioned, organized, oriented, coupled to, and/or related to each other in a patient. The osseous anatomy includes any aspects of each of the one or more bones of the anatomy of a patient. Osseous anatomy can refer to a single bone or a collection of bones for a particular anatomical structure including but not limited to a foot, an ankle, a leg, a knee, a hip, a spine, a hand, an elbow, an arm, a shoulder, a neck, and a skull. Osseous anatomy can refer to a healthy bone or bones, a collection of healthy and unhealthy bones, a collection of anatomically positioned bones and/or a collection of deformed bones, and the like.

As used herein, “patient-specific osteotomy procedure” refers to an osteotomy procedure that has been adjusted, tailored, modified, or configured to specifically address the anatomy, physiology, condition, abnormalities, needs, or desires of a particular patient. In certain aspects, one patient-specific osteotomy procedure may be useable in connection with only one patient. In other aspects, one patient-specific osteotomy procedure may be useable with a number of patients having a particular class of characteristics. In certain aspects, a patient-specific osteotomy procedure may refer to a non-patient-specific osteotomy procedure that includes one or more patient-specific implants and/or instrumentation. In another aspects, a patient-specific osteotomy procedure may refer to a patient-specific osteotomy procedure that includes one or more patient-specific implants, patient-specific surgical steps, and/or patient-specific instrumentation.

“Register” or “Registration” refers to an act of aligning, mating, contacting, engaging, or coupling one or more parts and/or surfaces of one object in relation to one or more parts and/or surfaces of another object. Often, the one or more parts and/or surfaces of one object include protrusions and/or depressions that are the inverse or mirror configuration of protrusions and/or depressions of one or more parts and/or surfaces of the other object.

“Remediation procedure” refers to any designed or performed for the purpose of remediating a condition of a patient and/or a condition of one or more parts of a body of a patient.

“Wedge osteotomy” refers to an osteotomy procedure in which one or more wedges are used as part of the procedure. Generally, wedge osteotomies can be of one of two types, open wedge and closing wedge. The type of osteotomy refers to how the procedure changes the relation between two parts of a bone involved in the osteotomy. In an open wedge osteotomy, a wedge of bone or graft or other material is inserted in between two parts of a bone. Consequently, a wedge shape is “opened” in the bone. In a close wedge osteotomy or closing wedge osteotomy a wedge of bone is removed from a bone. Consequently, a wedge shape formed in the bone is “closed.”

As used herein, an “opening” refers to a gap, a hole, an aperture, a port, a portal, a slit, a space or recess in a structure, a void in a structure, or the like. In certain embodiments, an opening can refer to a structure configured specifically for receiving something and/or for allowing access. In certain embodiments, an opening can pass through a structure. In such embodiments, the opening can be referred to as a window. In other embodiments, an opening can exist within a structure but not pass through the structure. In other embodiments, an opening can initiate on a surface or at an edge or at a side of a structure and extend into the structure for a distance, but not pass through or extend to another side or edge of the structure. In other embodiments, an opening can initiate on a surface or at an edge or at a side of a structure and extend into the structure until the opening extends through or extends to another side or edge of the structure. An opening can be two-dimensional or three-dimensional and can have a variety of geometric shapes and/or cross-sectional shapes, including, but not limited to a rectangle, a square, or other polygon, as well as a circle, an ellipse, an ovoid, or other circular or semi-circular shape. As used herein, the term “opening” can include one or more modifiers that define specific types of “openings” based on the purpose, function, operation, position, or location of the “opening.” As one example, a “fastener opening” refers to an “opening” adapted, configured, designed, or engineered to accept or accommodate a “fastener.”

“Hole” refers to a gap, an opening, an aperture, a port, a portal, a space or recess in a structure, a void in a structure, or the like. In certain embodiments, a hole can refer to a structure configured specifically for receiving something and/or for allowing access. In certain embodiments, a hole can pass through a structure. In other embodiments, an opening can exist within a structure but not pass through the structure. A hole can be two-dimensional or three-dimensional and can have a variety of geometric shapes and/or cross-sectional shapes, including, but not limited to a rectangle, a square, or other polygon, as well as a circle, an ellipse, an ovoid, or other circular or semi-circular shape. As used herein, the term “hole” can include one or more modifiers that define specific types of “holes” based on the purpose, function, operation, position, or location of the “hole.” As one example, a “fastener hole” refers to an “hole” adapted, configured, designed, or engineered to accept or accommodate a “fastener.” A “blind hole” is a hole with an opening on one side that does not extend all the way through a structure. In certain embodiments, a hole, including a blind hole, has a circular longitudinal cross-section. Alternatively, or in addition, a hole can have a cross-section of a variety of geometric shapes include a circle, an oval, a square, a rectangle, a slot with rounded ends, a triangle, or the like.

As used herein, “anatomic data” refers to data identified, used, collected, gathered, and/or generated in connection with an anatomy of a human or animal. Examples of anatomic data may include location data for structures, both independent, and those connected to other structures within a coordinate system. Anatomic data may also include data that labels or identifies one or more anatomical structures. Anatomic data can include volumetric data, material composition data, and/or the like. Anatomic data can be generated based on medical imaging data or measurements using a variety of instruments including monitors and/or sensors. Anatomic data can be gathered, measured, or collected from anatomical models and/or can be used to generate, manipulate, or modify anatomical models.

A bone model or anatomic model of a patient's body or body part(s) may be generated by computing devices that analyze medical imaging images. Structures of a patient's body can be determined using a process called segmentation.

“Set” refers to a collection of objects. A set can have zero or more objects in the collection. Generally, a set includes one or more objects in the collection.

“Trajectory guide” or “trajectory indicator” or “targeting guide” refers to any structure, apparatus, surface, device, system, feature, or aspect configured to indicate, identify, guide, place, position, or otherwise assist in marking or deploying a fastener or other structure along a desired trajectory for one or more subsequent steps in a procedure.

“Trajectory” refers to a path a body travels or a path configured for a body to travel through space. (Search “trajectory” on wordhippo.com. WordHippo, 2023. Web. Modified. Accessed 13 Jun. 2023.)

As used herein, a “guard” refers to an apparatus, instrument, structure, member, device, component, system, or assembly structured, organized, configured, designed, arranged, or engineered to prevent, limit, impede, stop, or restrict motion, action, or movement and/or operation of the another object, member, structure, component, part, apparatus, system, or assembly beyond a certain parameter such as a boundary. Said another way, a “guard” refers to an apparatus, instrument, structure, member, device, component, system, or assembly structured, organized, configured, designed, arranged, or engineered to retain, maintain, hold, keep, or restrict motion, action, or movement and/or operation of the another object, member, structure, component, part, apparatus, system, or assembly within or at one or more parameters such as a boundary.

As used herein, “artificial intelligence” refers to intelligence demonstrated by machines, unlike the natural intelligence displayed by humans and animals, which involves consciousness and emotionality. The distinction between artificial intelligence and natural intelligence categories is often revealed by the acronym chosen. ‘Strong’ AI is usually labelled as artificial general intelligence (AGI) while attempts to emulate ‘natural’ intelligence have been called artificial biological intelligence (ABI). Leading AI textbooks define the field as the study of “intelligent agents”: any device that perceives its environment and takes actions that maximize its chance of achieving its goals. The term “artificial intelligence” can also be used to describe machines that mimic “cognitive” functions that humans associate with the human mind, such as “learning” and “problem solving”. (Search “artificial intelligence” on Wikipedia.com Jun. 25, 2021. CC-BY-SA 3.0 Modified. Accessed Jun. 25, 2021.)

As used herein, “segmentation” or “image segmentation” refers to the process of partitioning an image into different meaningful segments. These segments may correspond to different tissue classes, organs, pathologies, bones, or other biologically relevant structures. Medical image segmentation accommodates imaging ambiguities such as low contrast, noise, and other imaging ambiguities.

Certain computer vision techniques can be used or adapted for image segmentation. For example, the techniques and or algorithms for segmentation may include, but are not limited to: Atlas-Based Segmentation: For many applications, a clinical expert can manually label several images; segmenting unseen images is a matter of extrapolating from these manually labeled training images. Methods of this style are typically referred to as atlas-based segmentation methods. Parametric atlas methods typically combine these training images into a single atlas image, while nonparametric atlas methods typically use all of the training images separately. Atlas-based methods usually require the use of image registration in order to align the atlas image or images to a new, unseen image.

Image registration is a process of correctly aligning images; Shape-Based Segmentation: Many methods parametrize a template shape for a given structure, often relying on control points along the boundary. The entire shape is then deformed to match a new image. Two of the most common shape-based techniques are Active Shape Models and Active Appearance Models; Image-Based Segmentation: Some methods initiate a template and refine its shape according to the image data while minimizing integral error measures, like the Active contour model and its variations; Interactive Segmentation: Interactive methods are useful when clinicians can provide some information, such as a seed region or rough outline of the region to segment. An algorithm can then iteratively refine such a segmentation, with or without guidance from the clinician. Manual segmentation, using tools such as a paint brush to explicitly define the tissue class of each pixel, remains the gold standard for many imaging applications. Recently, principles from feedback control theory have been incorporated into segmentation, which give the user much greater flexibility and allow for the automatic correction of errors; Subjective surface Segmentation: This method is based on the idea of evolution of segmentation function which is governed by an advection-diffusion model. To segment an object, a segmentation seed is needed (that is the starting point that determines the approximate position of the object in the image). Consequently, an initial segmentation function is constructed. With the subjective surface method, the position of the seed is the main factor determining the form of this segmentation function; and Hybrid segmentation which is based on combination of methods. (Search “medical image computing” on Wikipedia.com Jun. 24, 2021. CC-BY-SA 3.0 Modified. Accessed Jun. 24, 2021.)

As used herein, “medical imaging” refers to a technique and process of imaging the interior or exterior of a body for clinical analysis and medical intervention, as well as visual representation of the function of some organs or tissues (physiology). Medical imaging seeks to reveal internal structures hidden by the skin and bones, as well as to diagnose and treat disease. Medical imaging may be used to establish a database of normal anatomy and physiology to make possible identification of abnormalities.

Medical imaging in its widest sense, is part of biological imaging and incorporates radiology, which uses the imaging technologies of X-ray radiography, magnetic resonance imaging, ultrasound, endoscopy, elastography, tactile imaging, thermography, medical photography, nuclear medicine functional imaging techniques as positron emission tomography (PET) and single-photon emission computed tomography (SPECT). Another form of X-ray radiography includes computerized tomography (CT) scans in which a computer may control the position of the X-ray sources and detectors. Magnetic Resonance Imaging (MRI) is another medical imaging technology. Fluoroscopy is an imaging technique that uses X-rays to obtain real-time moving images of the interior of an object. In its primary application of medical imaging, a fluoroscope allows a physician to see the internal structure and function of a patient, so that the pumping action of the heart or the motion of swallowing, for example, can be watched. This is useful for both diagnosis and therapy and occurs in general radiology, interventional radiology, and image-guided surgery. (Search “medical imaging” on Wikipedia.com Jul. 14, 2021. CC-BY-SA 3.0 Modified. Accessed Sep. 1, 2021.)

Data analyzed, generated, manipulated, interpolated, collected, stored, reviewed, and/or modified in connection with medical imaging or medical image processing can be referred to herein as medical imaging data or medical image data.

Measurement and recording techniques that are not primarily designed to produce images, such as electroencephalography (EEG), magnetoencephalography (MEG), electrocardiography (ECG), and others, represent other technologies that produce data susceptible to representation as a parameter graph vs. time or maps that contain data about the measurement locations. In certain embodiments bone imaging includes devices that scan and gather bone density anatomic data. These technologies may be considered forms of medical imaging in certain disciplines. (Search “medical imaging” on Wikipedia.com Jun. 16, 2021. CC-BY-SA 3.0 Modified. Accessed Jun. 23, 2021.)

As used herein, As used herein, “patient imaging data” refers to data identified, used, collected, gathered, and/or generated in connection with medical imaging for a particular patient. Patient imaging data is one type of medical imaging data. Patient imaging data can be shared between users, systems, patients, and professionals using a common data format referred to as Digital Imaging and Communications in Medicine (DICOM) data. DICOM data is a standard format for storing, viewing, retrieving, and sharing medical images.

As used herein, “medical image computing” or “medical image processing” refers to systems, software, hardware, components, and/or apparatus that involve and combine the fields of computer science, information engineering, electrical engineering, physics, mathematics and medicine. Medical image computing develops computational and mathematical methods for working with medical images and their use for biomedical research and clinical care. One goal for medical image computing is to extract clinically relevant information or knowledge from medical images. While closely related to the field of medical imaging, medical image computing focuses on the computational analysis of the images, not their acquisition. The methods can be grouped into several broad categories: image segmentation, image registration, image-based physiological modeling, and others. (Search “medical image computing” on Wikipedia.com Jun. 24, 2021. CC-BY-SA 3.0 Modified. Accessed Jun. 24, 2021.) Medical image computing may include one or more processors or controllers on one or more computing devices. Such processors or controllers may be referred to herein as medical image processors. Medical imaging and medical image computing together can provide systems and methods to image, quantify and fuse both structural and functional information about a patient in vivo. These two technologies include the transformation of computational models to represent specific subjects/patients, thus paving the way for personalized computational models. Individualization of generic computational models through imaging can be realized in three complementary directions: definition of the subject-specific computational domain (anatomy) and related subdomains (tissue types); definition of boundary and initial conditions from (dynamic and/or functional) imaging; and characterization of structural and functional tissue properties. Medical imaging and medical image computing enable the translation of models to the clinical setting with both diagnostic and therapeutic applications. (Id.) In certain embodiments, medical image computing can be used to generate a bone model, a patient-specific model, and/or a patent specific instrument from medical imaging and/or medical imaging data.

As used herein, “model” refers to an informative representation of an object, person or system. Representational models can be broadly divided into the concrete (e.g. physical form) and the abstract (e.g. behavioral patterns, especially as expressed in mathematical form). In abstract form, certain models may be based on data used in a computer system or software program to represent the model. Such models can be referred to as computer models. Computer models can be used to display the model, modify the model, print the model (either on a 2D medium or using a 3D printer or additive manufacturing technology). The printed physical form of the model can be referred to as a 3D physical bone model. Computer models can also be used in environments with models of other objects, people, or systems. Computer models can also be used to generate simulations, display in virtual environment systems, display in augmented reality systems, or the like. Computer models can be used in Computer Aided Design (CAD) and/or Computer Aided Manufacturing (CAM) systems. Certain models may be identified with an adjective that identifies the object, person, or system the model represents. For example, a “bone” model is a model of a bone, and a “heart” model is a model of a heart. (Search “model” on Wikipedia.com Jun. 13, 2021. CC-BY-SA 3.0 Modified. Accessed Jun. 23, 2021.)

A “bone model” or “anatomic model” refers to a model of a bone of a person. The bone model may model a single bone or a plurality of bones. The modeled bone and/or bones may be positioned in standard anatomical form and/or may be positioned relative to other bones (e.g., models of bones) of a person such that the positions of the bones in the bone model are the same or substantially the same as corresponding bones of a person, such as a patient.

“3D physical bone model” refers to a bone model that is represented in a tangible, physical form and has three-dimensions. A 3D physical bone model may include or model a single bone or may include or model a plurality of bones. A 3D physical bone model that includes a plurality of bone models may be configured such that bone models of the plurality of bone models are positioned and/or oriented with the 3D physical bone model as corresponding bones are positioned and/or oriented in a subject of the 3D physical bone model (e.g., a patient).

A 3D physical bone model that includes a plurality of bone models may be referred to as an assembly of 3D physical bone models because each bone model of the assembly may also be a 3D physical bone model. In certain embodiments, 3D physical bone models of a 3D physical bone model may be coupled, connected, or related to each other in the same or a similar manner to how corresponding bones are coupled, connected, or related to each other in a subject for a 3D physical bone model.

“Position” refers to a place or location. (Search “position” on wordhippo.com. WordHippo, 2024. Web. Accessed 8 Jan. 2024.) A position may be defined in a virtual environment such as in a model or set of models defined by and presented by a computing device. In addition, a position may be a place or location in a tangible physical environment such as in a space, on land, within or on a system, assembly, component, a patient, or other structure.

“Original position” refers to a position before any actions are taken to change a position of a structure, object, device, apparatus, component, or system. An original position may be defined in a virtual environment such as in a model or set of models defined by and presented by a computing device. Alternatively, or in addition, an original position may be a position in, on, or part of a tangible physical object, such as bones of a foot in a patient. In certain embodiments, an original position is a deformed position. An original position can be contrasted with a predetermined position which may be a position planned to implement a correction, correct a structure's position from a deformed position to a corrected position.

A “deformed position” refers to an anatomical structure that is positioned to form, include, or is at least part of a deformity. A “corrected position” refers to an anatomical structure positioned to remediate, correct, eliminate, and/or overcome a deformity. A predetermined position can be a corrected position.

“Feedback” refers to a reactionary response to an action, a product, service, or task. (Search “feedback” on wordhippo.com. WordHippo, 2023. Web. Modified. Accessed 28 Aug. 2023.)

As used herein, “additive manufacturing” refers to a manufacturing process in which materials are joined together in a process that repeatedly builds one layer on top of another to generate a three-dimensional structure or object. Additive manufacturing may also be referred to using different terms including additive processes, additive fabrication, additive techniques, additive layer manufacturing, layer manufacturing, freeform fabrication, ASTM F2792 (American Society for Testing and Materials), and 3D printing. Additive manufacturing can build the three-dimensional structure or object using computer-controlled equipment that applies successive layers of the material(s) based on a three-dimensional model that may be defined using Computer Aided Design (CAD) software. Additive manufacturing can use a variety of materials including polymers, thermoplastics, metals, ceramics, biochemicals, and the like. Additive manufacturing may provide unique benefits, as an implant together with the pores and/or lattices can be directly manufactured (without the need to generate molds, tool paths, perform any milling, and/or other manufacturing steps).

“Palpable feedback” refers to a type of feedback that can be felt. In one embodiment, palpable feedback can refer to feedback that is readily noticeable, tangible, or easily felt or perceived. During certain medical procedures, such as an osteotomy, palpable feedback can include the tactile sensations experienced by the healthcare provider as they move bones or bone fragments. (© ChatGPT 3.5 Version, Modified, accessed chat.openai.com/chat Feb. 2, 2024). Often palpable feedback is feedback a user, such as a surgeon, feels as they perform one or more steps or actions in a surgical procedure.

“Three-dimensional surface” refers to a surface defined by a collection of points that have three coordinates (x, y, z), where each point represents a location in space. In a medical context, a three-dimensional surface can include a surface of an implant or instrument that is customized for a particular purpose. In certain embodiments, a three-dimensional surface may be customized to fit anatomy of a patient or to accommodate handling by a user (e.g., a handle). (© ChatGPT 3.5 Version, Modified, accessed chat.openai.com/chat Feb. 2, 2024). In certain embodiments, of the present disclosure, a three-dimensional surface may be a surface on a side of an instrument that is specifically configured or customized to fit or match anatomy of a patient.

“Repository” refers to any data source or dataset that includes data or content. In one embodiment, a repository resides on a computing device. In another embodiment, a repository resides on a remote computing or remote storage device. A repository may comprise a file, a folder, a directory, a set of files, a set of folders, a set of directories, a database, an application, a software application, content of a text, content of an email, content of a calendar entry, and the like. A repository, in one embodiment, comprises unstructured data. A repository, in one embodiment, comprises structured data such as a table, an array, a queue, a look up table, a hash table, a heap, a stack, or the like. A repository may store data in any format including binary, text, encrypted, unencrypted, a proprietary format, or the like.

As used herein, “bevel” refers to an edge of a structure that is not perpendicular to the faces of the piece, the edge has a slope or slant or angled profile and can refer to a sloped surface. Often a cutting tool such as a blade or tooth can have a beveled edge that facilitates the cutting edge in cutting into a target material. “bevel” and “chamfer” can be used interchangeably herein. (Search “bevel” on Wikipedia.com May 17, 2021. CC-BY-SA 3.0 Modified. Accessed Aug. 4, 2021; search “bevel” on Merriam-Webster.com. Merriam-Webster, 2021. Web. Accessed 4 Aug. 2021. Modified; search “bevel” on wordhippo.com. WordHippo, 2021. Web. Accessed 4 Aug. 2021. Modified.)

As used herein, “registration” or “image registration” refers to a method, process, module, component, apparatus, and/or system that seeks to achieve precision in the alignment of two images. As used here, “image” may refer to either or both an image of a structure or object and another image or a model (e.g., a computer-based model or a physical model, in either two dimensions or three dimensions). In the simplest case of image registration, two images are aligned. One image may serve as the target image and the other as a source image; the source image is transformed, positioned, realigned, and/or modified to match the target image. An optimization procedure may be applied that updates the transformation of the source image based on a similarity value that evaluates the current quality of the alignment. An iterative procedure of optimization may be repeated until a (local) optimum is found. An example is the registration of CT and PET images to combine structural and metabolic information. Image registration can be used in a variety of medical applications: Studying temporal changes; Longitudinal studies may acquire images over several months or years to study long-term processes, such as disease progression. Time series correspond to images acquired within the same session (seconds or minutes). Time series images can be used to study cognitive processes, heart deformations and respiration; Combining complementary information from different imaging modalities. One example may be the fusion of anatomical and functional information.

Since the size and shape of structures vary across modalities, evaluating the alignment quality can be more challenging. Thus, similarity measures such as mutual information may be used; Characterizing a population of subjects. In contrast to intra-subject registration, a one-to-one mapping may not exist between subjects, depending on the structural variability of the organ of interest. Inter-subject registration may be used for atlas construction in computational anatomy. Here, the objective may be to statistically model the anatomy of organs across subjects; Computer-assisted surgery: in computer-assisted surgery pre-operative images such as CT or MRI may be registered to intra-operative images or tracking systems to facilitate image guidance or navigation. There may be several considerations made when performing image registration: The transformation model. Common choices are rigid, affine, and deformable transformation models. B-spline and thin plate spline models are commonly used for parameterized transformation fields. Non-parametric or dense deformation fields carry a displacement vector at every grid location; this may use additional regularization constraints. A specific class of deformation fields are diffeomorphisms, which are invertible transformations with a smooth inverse; The similarity metric. A distance or similarity function is used to quantify the registration quality. This similarity can be calculated either on the original images or on features extracted from the images. Common similarity measures are sum of squared distances (SSD), correlation coefficient, and mutual information. The choice of similarity measure depends on whether the images are from the same modality; the acquisition noise can also play a role in this decision. For example, SSD may be the optimal similarity measure for images of the same modality with Gaussian noise. However, the image statistics in ultrasound may be significantly different from Gaussian noise, leading to the introduction of ultrasound specific similarity measures.

Multi-modal registration may use a more sophisticated similarity measure; alternatively, a different image representation can be used, such as structural representations or registering adjacent anatomy; The optimization procedure. Either continuous or discrete optimization is performed. For continuous optimization, gradient-based optimization techniques are applied to improve the convergence speed. (Search “medical image computing” on Wikipedia.com Jun. 24, 2021. CC-BY-SA 3.0 Modified. Accessed Jun. 25, 2021.)

As used herein, a “resection” refers to a method, procedure, or step that removes tissue from another anatomical structure or body. A resection is typically performed by a surgeon on a part of a body of a patient. (Search “surgery” on Wikipedia.com May 26, 2021. CC-BY-SA 3.0 Modified. Accessed May 26, 2021.) In certain embodiments, a resection may remove little or no tissue and may in such circumstances also be referred to as an incision or a dissection. Resection may be used as a noun or a verb. In the verb form, the term is “resect” and refers to an act of performing, or doing, a resection. Past tense of the verb resect is resected.

“Resection feature” refers to any feature configured, designed, engineered and/or intended to facilitate resection. Examples of a resection guide feature include but are not limited to, a slot, a cut channel, a cut slot, a pivoting cut guide, a pivoting resection guide, an opening, a straight slot, an angled slot, a curved slot, or the like.

“Patient-matched” refers to a feature, aspect, attribute, characteristic, instrument, and/or device that is selected from a set of predetermined, predefined, precalculated, preconfigured, prearranged, and/or pre-fabricated structures, apparatuses, devices, instruments or devices to satisfactorily service a user based on a set of characteristics, such as size of an anatomical structure, deformity, fracture, laceration, opening, angles for certain landmarks, angles for a deformity, type of deformity, size of the bone, and the like. In certain embodiments, patient-matched is different from patient-specific.

“Malrotation” refers to a condition in which a part, typically a part of a patient's body has rotated from a normal position to an unnormal or uncommon position.

As used herein, a “guide” refers to a part, component, member, or structure designed, adapted, configured, or engineered to guide or direct one or more other parts, components, or structures. A guide may be part of, integrated with, connected to, attachable to, or coupled to, another structure, device, or instrument. In one embodiment, a guide may include a modifier that identifies a particular function, location, orientation, operation, type, and/or a particular structure of the guide. Examples of such modifiers applied to a guide, include, but are not limited to, “pin guide” that guides or directs one or more pins, a “cutting guide” that guides or directs the making or one or more cuts, a placement, deployment, or insertion guide that guides or directs the placement, positioning, orientation, deployment, installation, or insertion of a fastener and/or implant, a “cross fixation guide” that guides deployment of a fastener or fixation member, an “alignment guide” that guides the alignment of two or more objects or structures, a “resection guide” that serves to guide resection of soft or hard tissue, such as in an osteotomy, a “reduction guide” can serve to guide reduction of one or more bone segments or fragments, an “placement guide” that serves to identify how an object can be placed in relation to another object or structure, and the like. Furthermore, guides may include modifiers applied due to the procedure or location within a patient for which the guide is to be used. For example, where a guide is used at a joint, the guide may be referred to herein as an “arthrodesis guide”.

“Guide pin” refers to a pin, structure, or a type of fastener that can be used to guide an instrument or implant as part of a method, process, or procedure, such as a surgical technique. In certain aspects, a guide pin may be designed for temporary use until subsequent steps in a method, process, or procedure. Examples of a guide pin include, but are not limited to, a pin, a K-wire, and the like.

“Straight cut” is a type of cut that may be used for a surgical procedure. Generally, a straight cut is a cut in tissue (soft tissue or hard tissue) that is perpendicular to a surface where the cut is made and extends within and/or through the tissue along a straight line. Advantageously, straight cuts may be easier for a surgeon to perform than an angled cut or a curved cut. In certain embodiments, a straight cut is made with a guide (e.g., cut guide, resection guide, and/or resection feature). Alternatively, or in addition, a user may make a straight cut free-hand (without the aid of a guide, instrument, or instrumentation).

As used herein, “feature” refers to a distinctive attribute or aspect of something. (Search “feature” on google.com. Oxford Languages, 2021. Web. 20 Apr. 2021.) A feature may include one or more apparatuses, structures, objects, systems, sub-systems, devices, or the like. A feature may include a modifier that identifies a particular function or operation and/or a particular structure relating to the feature. Examples of such modifiers applied to a feature, include, but are not limited to, “attachment feature,” “securing feature,” “placement feature,” “protruding feature,” “engagement feature,” “disengagement feature,” “resection feature”, “guide feature”, and the like.

“Engagement feature” or “Engagement member” refers to an apparatus, instrument, structure, device, component, member, system, assembly or module structured, organized, configured, designed, arranged, or engineered to connect, join, link, couple to, or engage with another object, apparatus, instrument, structure, device, component, member, system, assembly or module either permanently or temporarily. The connection, coupling, linkage, or engagement may be a mechanical connection or interconnection.

“Cortical bone” refers to a type of bone tissue. Cortical bone is a type of bone tissue typically found between an external surface of a bone and an interior area of the bone. Cortical bone is more dense and typically stronger structurally than other types of bone tissue. “Cortical surface” refers to a surface of cortical bone.

“Transosseous placement feature” refers to a placement feature that extends through one or more bones and that enables, or facilitates, placement of another device, apparatus, or instrument.

“Patient-specific” refers to an attribute, aspect, feature, characteristic, function, structure, device, guide, tool, instrument, apparatus, member, component, system, assembly, module, or subsystem that is adjusted, tailored, modified, organized, configured, designed, arranged, engineered, and/or fabricated to specifically address the anatomy, physiology, condition, abnormalities, needs, or desires of a particular patient and/or surgeon serving the particular patient. In one aspect, a patient specific aspect or feature is unique to a single patient and may include features unique to the patient such as a number of cut channels, a number of bone attachment features, a number of bone engagement surfaces, a number of resection features, a depth of one or more cutting channels, an angle for one or more resection channels, a surface contour, component position, component orientation, and/or other features.

“Patient specific feature” refers to a feature, function, structure, device, guide, tool, instrument, apparatus, member, component, system, assembly, module, or subsystem that is adjusted, tailored, modified, organized, configured, designed, arranged, engineered, and/or fabricated to specifically address the anatomy, physiology, condition, abnormalities, needs, or desires of a particular patient or surgeon serving the particular patient. In one aspect, a patient specific feature is unique to a single patient and may include features unique to the patient such as a number of cut channels, a number of bone attachment features, a number of bone engagement surfaces, a number of resection features, a depth of one or more cutting channels, an angle for one or more resection channels, a surface contour, component position, component orientation, and/or other features. “Medial resection guide” refers to a resection guide designed, engineered, fabricated, or intended for use with, one, in, or about a medial part, section, surface, portion, or aspect of an anatomical structure such as a bone, digit, limb, or other anatomical structure for one or more steps of a resection procedure. “Lateral resection guide” refers to a resection guide designed, engineered, fabricated, or intended for use with, one, in, or about a lateral part, section, surface, portion, or aspect of an anatomical structure such as a bone, digit, limb, or other anatomical structure for one or more steps of a resection procedure.

“Bone fragment” refers to a part of a bone that is normally part of another bone of a patient. A bone fragment may be separate from another bone of a patient due to a deformity or trauma. In one aspect, the bone the bone fragment is normally connected or joined with is referred to as a parent bone.

“Orientation” refers to a direction, angle, position, condition, state, or configuration of a first object, component, part, apparatus, system, or assembly relative to another object, component, part, apparatus, system, assembly, reference point, reference axis, or reference plane.

“Longitudinal axis” refers to an axis of a structure, device, object, apparatus, or part thereof that extends from one end of a longest dimension to an opposite end. Typically, a longitudinal axis passes through a center of the structure, device, object, apparatus, or part thereof along the longitudinal axis. The center point used for the longitudinal axis may be a geometric center point and/or a mass center point.

Those of skill in the art will appreciate that a resection feature may take a variety of forms and may include a single feature or one or more features that together form the resection feature. In certain embodiments, the resection feature may take the form of one or more slots. Alternatively, or in addition, a resection feature may be referenced using other names including, but not limited to, channel, cut channels, and the like.

“Cutting tool” refers to any tool that can be used to cut or resect another object. In particular, a cutting tool can refer to a manual or power tool for cutting or resecting tissue of a patient. Examples of cutting tools include, but are not limited to, a burr, an oscillating saw, a reciprocating saw, a grater saw, a drill, a mill, a side-cutting burr, or the like.

“Revised model” or “Modified model” or “Corrected model” refers to a model that has been changed from an original condition to an altered or changed or revised condition. Generally, an original model is used to create the revised/corrected model. Alternatively, a revised model can be generated from scratch. A revised model can include a computer model and/or a physical tangible 3D model. Often the original model exists in a digital form on a computer. Such models can be referred to as CAD models.

“Corrected model” refers to a model in which an object, structure, and/or subject of the model has been changed from a deformed or incorrect configuration to a corrected or revised configuration. A corrected model can be generated from scratch or can be generated by revising an existing model and/or merging two or more models. As one example, an original model may represent one or more bones of a foot. The modeled bones of the foot may be subject to a bone condition and therefore have a deformity. A corrected model can be created or formed from the original model by revising or changing one or more aspects of the original model such that the modeled bones reflect a corrected orientation and or configuration for the modeled bones.

“Prescription” or “Prescribed” refers to an instruction, request, direction, determination, designation, authorization, and/or order, as by a physician or nurse practitioner, for the administration of a medicine, preparation of an implant, preparation of an instrument, preparation of a model, or other intervention. Often a prescription is written. Prescription can also refer to the prescribed medicine or intervention. (Search “prescription” on wordhippo.com. WordHippo, 2023. Web. Accessed 3 May 2023. Modified.)

“User input” refers to any signal, action, or other indication from a user that provides direction, instruction(s), and/or information a user wants to provide to a device, apparatus, member, component, system, assembly, module, subsystem, circuit. In certain embodiments, user input can include input data provided by a user or operator. In certain embodiments, a user may provide user input using an input device such as a touchscreen, a mouse, a switch, a lever or the like. A variety of signals, indicators, indications, gestures, movements, touches, keystrokes, or the like can serve as user input.

The present disclosure discloses methods, systems, and/or apparatuses for providing a physical model of a patient anatomical structure to a customer. In one embodiment, the physical model is a three-dimensional model of the patient anatomical structure. In certain embodiments, the patient anatomical structure is a foot, a foot and ankle, a hand, a hand and wrist, a shoulder, a knee, a neck or the like.

Medical care and medical technology continues to advance. Surgeons continue to find new ways to address patient's needs while minimizing the risks of an adverse outcome, the pain and discomfort for the patient, and the length of recovery while increasing the likelihood of desired outcomes. In particular, surgeons continue to work to make smaller incisions and perform surgical procedures in smaller spaces by way of minimally invasive surgical (MIS) procedures.

While MIS procedures can provide advantages for the patient, they can increase stress or present other challenges for the surgeon. What is needed is a tool that would provide a surgeon with an accurate representation of the hard tissue and/or soft tissue of a patient before, during, and/or a surgical procedure. Modern technologies enable a surgeon to visualize anatomical structures of a particular patient using two dimensional pictures, images, on paper or video screens. Other technologies enable a surgeon to see three dimensional representations of anatomical structures, again on computer screens or using augmented reality technologies.

While such technologies can be helpful, they are not the same and do not provide the same advantages as providing a surgeon with a physical three-dimensional model of one or more anatomical structures of a particular patient. Embodiments of the present disclosure are a physical three-dimensional model of one or more anatomical structures of a particular patient to a customer, such as the patient, a facility, and/or a surgeon.

The present disclosure describes apparatuses, systems, and/or methods for generating and/or providing both patient-specific physical three-dimensional models and/or patient-specific instrumentation including instruments, guides, implants, and the like.

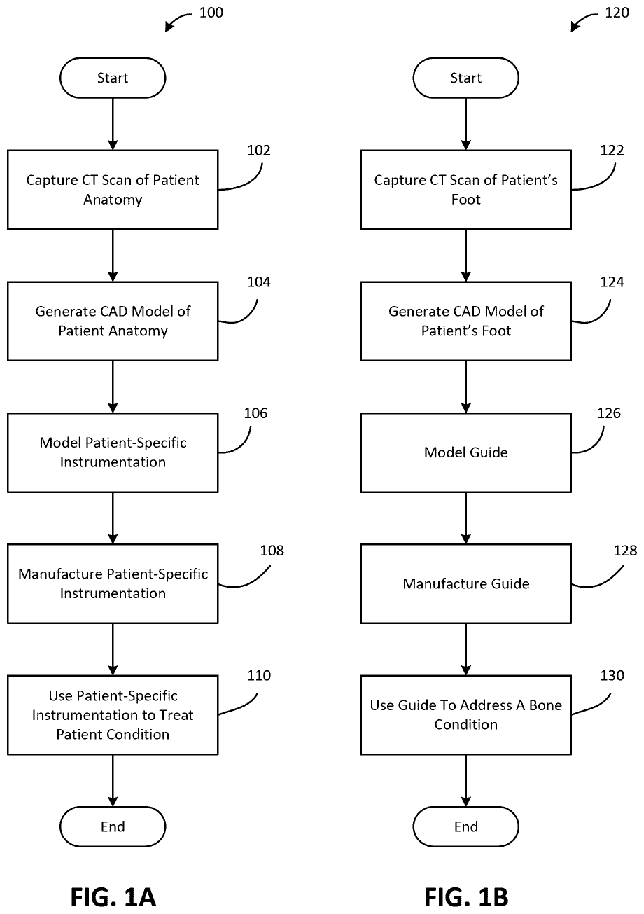

FIG. 1A is a flowchart diagram depicting a method 100 for correcting a bone condition, according to one embodiment. The method 100 may be used for any of a wide variety of bone conditions, including but not limited to deformities, fractures, joint failure, and/or the like. Further, the method 100 may provide correction with a wide variety of treatments, including but not limited to arthroplasty, arthrodesis, fracture repair, and/or the like.

As shown, the method 100 may begin with a step 102 in which a CT scan (or another three-dimensional image, also referred to as medical imaging) of the patient's anatomy is obtained. The step 102 may entail capturing a scan of only the particular bone(s) to be treated, or may entail capture of additional anatomic information, such as the surrounding tissues. Additionally, or alternatively, the step 102 may entail receiving a previously captured image, for example, at a design and/or fabrication facility. Performance of the step 102 may result in possession of a three-dimensional model of the patient's anatomy, or three-dimensional surface points that can be used to construct such a three-dimensional model.

After the step 102 has been carried out, the method 100 may proceed to a step 104 in which a CAD model of the patient's anatomy (including one or more bones) is generated. The CAD model may be one example of a bone model. The CAD model may be of any known format, including but not limited to SolidWorks, Catia, AutoCAD, or DXF. In some embodiments, customized software may be used to generate the CAD model from the CT scan. The CAD model may only include the bone(s) to be treated and/or may include surrounding tissues. In alternative embodiments, the step 104 may be omitted, as the CT scan may capture data that can directly be used in future steps without the need for conversion.

In one embodiment, the CAD model generated and/or patient-specific instrumentation, implants, and/or plan for conducting an operative procedure, may be enhanced by the use of advanced computer analysis system, machine learning, and/or automated/artificial intelligence. For example, these technologies may be used to revise a set of steps for a procedure such that a more desirable outcome is achieved.

In a step 106, the CAD model and/or CT scan data may be used to model patient-specific instrumentation that can be used to correct the condition, as it exists in the patient's anatomy. In some embodiments, any known CAD program may be used to view and/or manipulate the CAD model and/or CT scan and generate one or more instruments that are matched specifically to the size and/or shape of the patient's bone(s). In some embodiments, such instrumentation may include a targeting guide, trajectory guide, drill guide, cutting guide, tendon trajectory guide, positioner or positioning guide or similar guide that can be attached to one or more bones, with one or more features that facilitate work on the one or more bones pursuant to a procedure such as arthroplasty or arthrodesis. In some embodiments, performance of the step 106 may include modelling an instrument with a bone engagement surface that is shaped to match the contour of a surface of the bone, such that the bone engagement surface can lie directly on the corresponding contour.

In a step 108, the model(s) may be used to manufacture patient-specific instrumentation and/or implants. This may be done via any known manufacturing method, including casting, forging, milling, additive manufacturing, and/or the like. Additive manufacturing may provide unique benefits, as the model may be directly used to manufacture the instrumentation and/or implants (without the need to generate molds, tool paths, and/or the like beforehand). Such instrumentation may optionally include a targeting guide, trajectory guide, drill guide, cutting guide, positioning guide, positioner, or tendon trajectory guide with the bone engagement surface and one or more features as described herein.

In addition to, or in the alternative to the step 108, the model(s) may be used to select from available sizes of implants and/or instruments and advise the surgeon accordingly. For example, where a range of guides are available for a given procedure, analysis of the CAD data may facilitate pre-operative selection of the optimal guide and/or optimal placement of the guide on the bone. Similarly, if a range of implants may be used for a given procedure, analysis of the CAD data may facilitate pre-operative selection of the optimal implant(s). More particularly, properly-sized spacers, screws, bone plates, and/or other hardware may be pre-operatively selected.

Thus, the result of the step 108 may provision, to the surgeon, of one or more of the following: (1) one or more patient-specific instruments; (2) one or more patient-specific implants; (3) an instrument, selected from one or more available instrument sizes and/or configurations; (4) an implant, selected from one or more available implant sizes and/or configurations; (5) instructions for which instrument(s) to select from available instrument sizes and/or configurations; (6) instructions for which implant(s) to select from available implant sizes and/or configurations; (7) instructions for proper positioning or anchorage of one or more instruments to be used in the procedure; and (8) instructions for proper positioning or anchorage of one or more implants to be used in the procedure. These items may be provided to the surgeon directly, or to a medical device company or representative, for subsequent delivery to the surgeon.

In a step 110, the manufactured instrumentation may be used in surgery to facilitate treatment of the condition. In some embodiments, this may entail placing the modelled bone engagement surface against the corresponding contour of the bone used to obtain its shape, and then using the resection feature(s) to guide resection of one or more bones. Then the bone(s) may be further treated, for example, by attaching one or more joint replacement implants (in the case of joint arthroplasty), or by attaching bone segments together (in the case of arthrodesis or fracture repair). Prior to completion of the step 110, the instrumentation may be removed from the patient, and the surgical wound may be closed.

As mentioned previously, the method 100 may be used to correct a wide variety of bone conditions. One example of the method 100 will be shown and described in connection with FIG. 1B, for correction of a bunion deformity of the foot.

In certain embodiments, one or more of a method, apparatus, and/or system of the disclosed solution can be used for training a surgeon to perform a patient-specific procedure or technique. In one embodiment, the CAD model generated and/or patient-specific instrumentation, implants, and/or plan for conducting an operative procedure can be used to train a surgeon to perform a patient-specific procedure or technique.

In one example embodiment, a surgeon may submit a CT scan of a patient's foot to an apparatus or system that implements the disclosed solution. Next, a manual or automated process may be used to generate a CAD model and for making the measurements and correction desired for the patient. In the automated process, advanced computer analysis system, machine learning and automated/artificial intelligence may be used to generate a CAD model and/or one or more patient-specific instruments and/or operation plans. For example, a patient-specific guide may be fabricated that is registered to the patient's anatomy using a computer-aided machine (CAM) tool. In addition, a CAM tool may be used to fabricate a 3D structure representative of the patient's anatomy, referred to herein as a patient-specific synthetic cadaver. (e.g. one or more bones of a patient's foot). Next, the patient-specific guide and the patient-specific synthetic cadaver can be provided to a surgeon who can then rehearse an operation procedure in part or in full before going into an operating room with the patient.

In certain embodiments, the patient-specific guide or instrument can be used to preposition and/or facilitate pre-drilling holes for a plate system for fixation purposes. Such plate systems may be optimally placed, per a CT scan, after a correction procedure for optimal fixation outcome. In another embodiment, the CAD model and/or automated process such as advanced computer analysis, machine learning and automated/artificial intelligence may be used to measure a depth of the a through a patient-specific resection guide for use with robotics apparatus and/or systems which would control the depth of each cut within the guide to protect vital structures below or adjacent to a bone being cut. In another embodiment, the CAD model and/or automated process such as advanced computer analysis, machine learning and automated/artificial intelligence may be used to define desired fastener (e.g. bone screw) length and/or trajectories through a patient-specific guide and/or implant. The details for such lengths, trajectories, and components can be detailed in a report provided to the surgeon preparing to perform a procedure.

FIG. 1B is a flowchart diagram depicting a method 120 for correcting or remediating a bone condition, according to one embodiment. The method 120 may be used to prepare for an orthopedic procedure which corrects or remediates a bone, muscle, and/or tendon condition of a patient.

As shown, the method 120 may begin with a step 122 in which a CT scan (or another three-dimensional image) of the patient's foot is obtained. The step 122 may entail capturing a scan of only the first cuneiform and first metatarsal, or may entail capture of additional anatomic information, such as the entire foot. Additionally, or alternatively, the step 122 may entail receipt of previously captured image data. Capture of the entire foot in the step 122 may facilitate proper alignment of the first metatarsal with the rest of the foot (for example, with the second metatarsal). Performance of the step 122 may result in generation of a three-dimensional model of the patient's foot, or three-dimensional surface points that can be used to construct such a three-dimensional model.

After the step 122 has been carried out, the method 120 may proceed to a step 124 in which a CAD model of the relevant portion of the patient's anatomy is generated. The CAD model may optionally include the bones of the entire foot, like the CT scan obtained in the step 122. In alternative embodiments, the step 124 may be omitted in favor of direct utilization of the CT scan data, as described in connection with the step 104.

In a step 126, the CAD model and/or CT scan data may be used to model patient-specific instrumentation that can be used to correct or remediate a bone condition. Such instrumentation may include a guide. In one example, the guide can seat or abut or contact a surface of a bone and including an opening that guides a trajectory for a fastener for a procedure. In some embodiments, performance of the step 126 may include modelling the guide with a bone engagement surface that is shaped to match contours of the surfaces of the bone, such that the bone engagement surface can lie directly on the corresponding contours of the bone.

In a step 128, the model(s) may be used to manufacture patient-specific instrumentation and/or instruments. This may include manufacturing the guide with the bone engagement surface and/or other features as described above. As in the step 108, the step 128 may additionally or alternatively involve provision of one or more instruments and/or implants from among a plurality of predetermined configurations or sizes. Further, the step 128 may additionally, or alternatively, involve provision of instructions for placement and/or anchorage of one or more instruments and/or instruments to carry out the procedure.

In a step 130, the manufactured guide may be used in surgery to facilitate treatment of the condition. Specifically, the bone engagement surface of the guide may be placed against the corresponding contours of the bone. The guide may include an opening and/or trajectory guide to guide insertion of a trajectory guide such as a temporary fastener such as a K-wire. The guide may then be removed, and the remaining steps of a surgical procedure performed.

The method 100 and the method 120 are merely exemplary. Those of skill in the art will recognize that various steps of the method 100 and the method 120 may be reordered, omitted, and/or supplemented with additional steps not specifically shown or described herein.

As mentioned previously, the method 120 is one species of the method 100; the present disclosure encompasses many different procedures, performed with respect to many different bones and/or joints of the body. Exemplary steps and instrumentation for the method 120 will further be shown and described in connection with the present disclosure. Those of skill in the art will recognize that the method 120 may be used in connection with different instruments; likewise, the instruments of the present disclosure may be used in connection with methods different from the method 100 and the method 120.

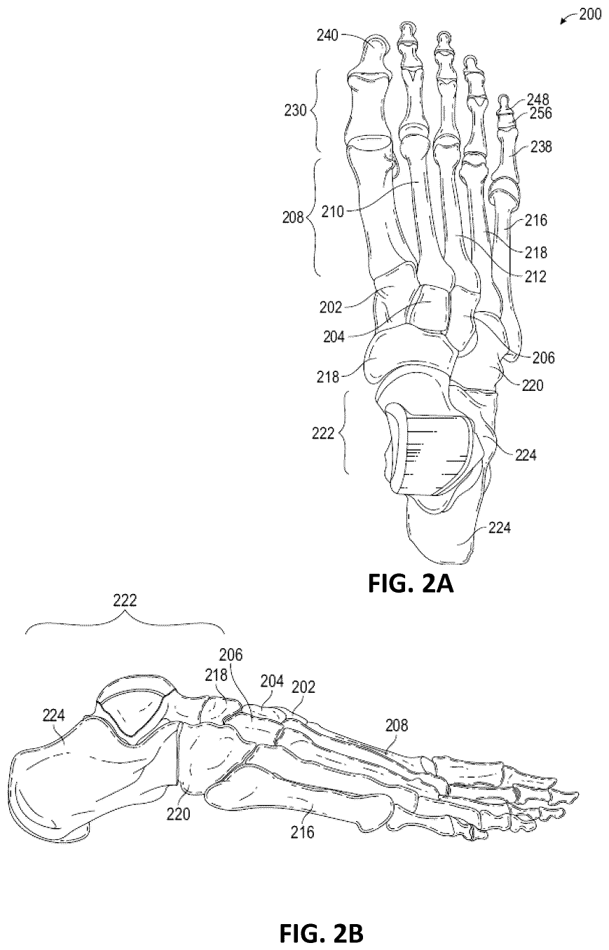

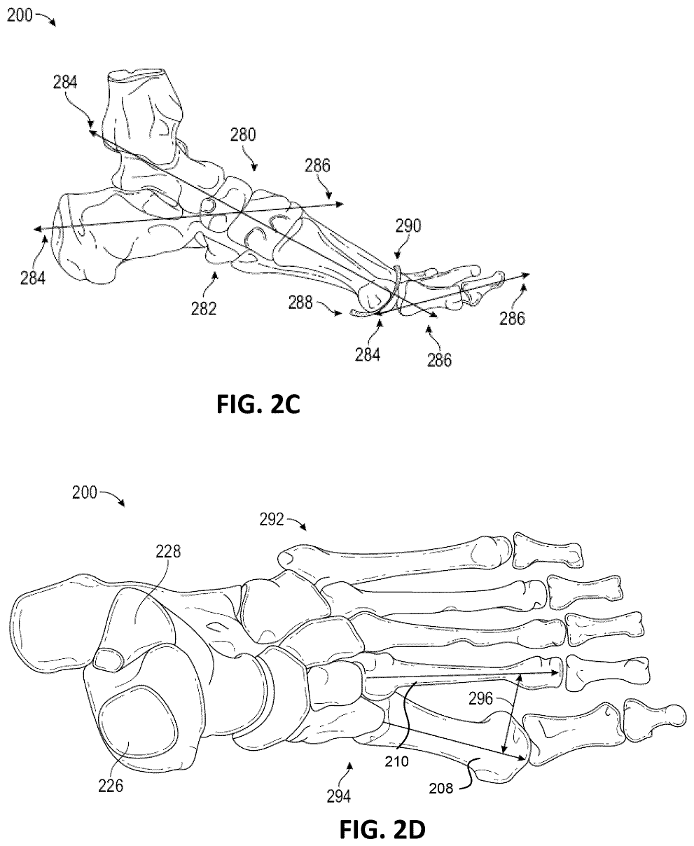

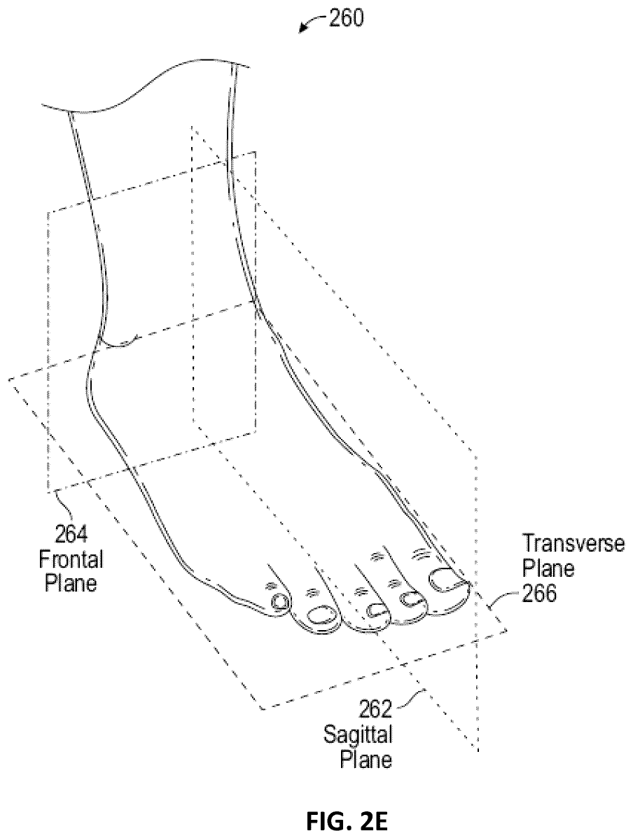

FIG. 2A is a perspective dorsal view of a foot 200. The foot 200 may have a medial cuneiform 202, an intermediate cuneiform 204, lateral cuneiform 206, a first metatarsal 208, a second metatarsal 210, third metatarsal 212, fourth metatarsal 214, fifth metatarsal 216, navicular 218, cuboid 220, and calcaneus 222, among others. The medial cuneiform 202 and the intermediate cuneiform 204 may be joined together at a first metatarsocuneiform joint, and the first metatarsal 208 and the second metatarsal 210 may be joined together at a second metatarsocuneiform joint. The foot 200 includes a set of proximal phalanges numbered first through fifth (230, 232, 234, 236, 238) and a set of distal phalanges numbered first through fifth (240, 242, 244, 246, 248) and a set of middle phalanges numbered second through fifth (250, 252, 254, 256).

FIG. 2B is a perspective lateral view of a foot 200, with bones of the foot labeled.

FIG. 2C is a perspective medial view of a foot illustrating a dorsal side 280 and a plantar side 282. Dorsal refers to the top of the foot. Plantar refers to the bottom of the foot. Proximal 284 is defined as “closer to the primary attachment point”. Distal 286 is defined as “further away from the attachment point”. Plantarflex or plantarflexion 288 means movement toward the plantar side 282 of a foot or hand, toward the sole or palm. Dorsiflex or dorsiflexion 290 means movement toward the dorsal side 280 of a foot or hand, toward the top. FIG. 2D is a perspective dorsal view of the foot 200. A transverse plane is the plane that shows the top of the foot. A lateral side 292 means a side furthest away from the midline of a body, or away from a plane of bilateral symmetry of the body. A medial side 294 means a side closest to the midline of a body, or toward a plane of bilateral symmetry of the body. For a Lapidus procedure, the intermetatarsal (IM) angle 296 is the angle to be corrected to remove the hallux valgus (bunion) deformity. The IM angle 296 is an angle between a longitudinal axis of a first metatarsal and a second metatarsal of a patient.

FIG. 2E is a view of a foot illustrating common planes 260 of reference for a human foot. FIG. 2E illustrates a sagittal plane 262 that divides the foot into a right section and a left section half. The sagittal plane 262 is perpendicular to frontal or coronal plane 264 and the transverse plane 266. In the foot, the frontal plane 264 generally runs vertically through the ankle and the transverse plane 266 generally runs horizontally through the midfoot and toes of the foot.

Every patient and/or condition is different; accordingly, the degree of angular adjustment needed in each direction may be different for every patient. Use of a patient-specific guide may help the surgeon obtain an optimal realignment, target, or position a bone tunnel, position one or more resections and/or fasteners and the like. Thus, providing patient-specific guides, jigs, and/or instrumentation may provide unique benefits.

The present patient-specific instrumentation may be used to correct a wide variety of bone conditions. Such conditions include, but are not limited to, any angular deformities from within one bone segment in either the lower or upper extremities (for example, tibial deformities, calcaneal deformities, femoral deformities, and radial deformities). The present disclosure may also be used to treat an interface between two bone segments (for example, the ankle joint, metatarsal cuneiform joint, lisfranc's joint, complex Charcot deformity, wrist joint, knee joint, etc.). As one example, an angular deformity or segmental malalignment in the forefoot may be treated, such as is found at the metatarsal cuneiform level, the midfoot level such as the navicular cuneiform junction, hindfoot at the calcaneal cuboid or subtalar joint or at the ankle between the tibia and talar junction. Additionally, patient-specific instruments could be used in the proximal leg between two bone segments or in the upper extremity such as found at the wrist or metacarpal levels.

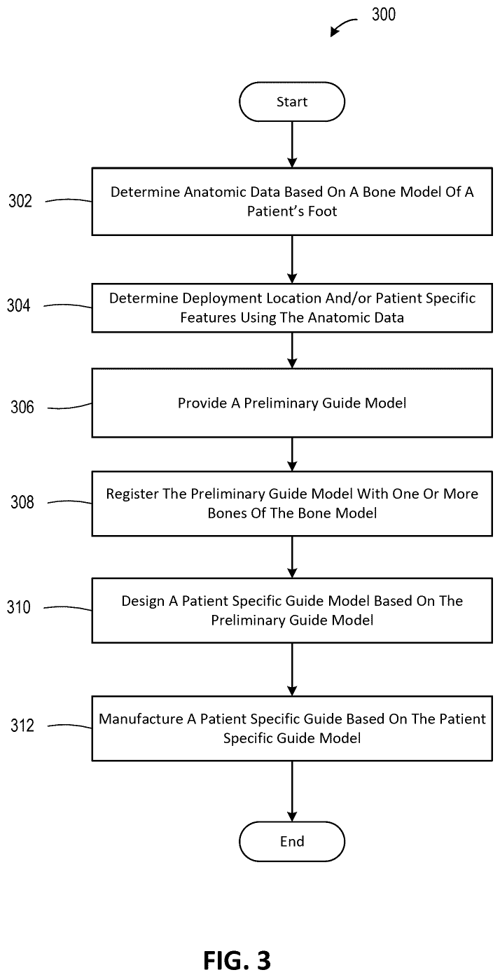

FIG. 3 illustrates a flowchart diagram depicting a method 300 for generating one or more patient-specific instruments configured to correct or address a bone or foot condition, according to one embodiment. Prior to steps of the method 300, a bone model (also referred to as CAD model above) is generated. The bone model may be generated using medical imaging of a patient's foot and may also be referred to as an anatomic model. The medical imaging image(s) may be used by computing devices to generate patient imaging data. The patient imaging data may be used to measure and account for orientation of one or more structures of a patient's anatomy. In certain embodiments, the patient imaging data may serve, or be a part of, anatomic data for a patient.

In one embodiment, the method 300 begins after a bone model of a patient's body or body part(s) is generated. In a first step 302, the method 300 may review the bone model and data associated with the bone model to determine anatomic data of a patient's foot.

After step 302, the method 300 may determine 304 a recommended location and/or a trajectory angle and/or patient-specific features for a procedure using the anatomic data. “Recommended location” refers to a location for deployment of guide or instrument on, in, between, or within one or more body parts (e.g., bones) of a patient. “Trajectory angle” refers to a recommended angle for deployment of an instrument, graft, body part, or resection feature angle relative to a bone of a patient for a procedure. In certain embodiments, determining the recommended location may employ advanced computer analysis system, expert systems, machine learning, and/or automated/artificial intelligence. In another embodiment, the method 300 may include determining one or more alternative locations and/or trajectory angles for instrumentation.

Next, the method 300 may proceed and a preliminary guide model is provided 306 from a repository of template instrumentation models. A preliminary guide model is a model of a preliminary guide.

As used herein, “preliminary guide” refers to a guide configured, designed, and/or engineered to serve as a template, prototype, archetype, or starting point for creating, generating, or fabricating a patient-specific guide. In one aspect, the preliminary guide may be used, as-is, without any further changes, modifications, or adjustments and thus become a patient-specific guide. In another aspect, the preliminary guide may be modified, adjusted, or configured to more specifically address the goals, objectives, or needs of a patient or a surgeon and by way of the modifications become a patient-specific guide. The patient-specific guide can be used by a user, such as a surgeon, to guide steps in a surgical procedure, such as an osteotomy. Accordingly, a preliminary guide model can be used to generate a patient-specific guide. The patient-specific guide model may be used in a surgical procedure to facilitate one or more steps of the procedure and may be used to generate a patient-specific guide that can be used in a surgical procedure for the patient.

In certain embodiments, the preliminary guide model may be generated based on anatomic data and/or a bone model or a combination of these, and no model or predesigned structure, template, or prototype. Alternatively, or in addition, the preliminary guide model may be, or may originate from, a template guide model selected from a set of template guide models. Each model in the set of template guide models may be configured to fit an average patient's foot. The template guide model may subsequently be modified or revised by an automated process or manual process to generate the preliminary guide model used in this disclosure.

As used herein, “template guide” refers to a guide configured, designed, and/or engineered to serve as a template for creating, generating, or fabricating a patient-specific guide. In one aspect, the template guide may be used, as-is, without any further changes, modifications, or adjustments and thus become a patient-specific guide. In another aspect, the template guide may be modified, adjusted, or configured to more specifically address the goals, objectives, or needs of a patient or a surgeon and by way of the modifications become a patient-specific guide. The patient-specific guide can be used by a user, such as a surgeon, to guide making one or more resections of a structure, such as a bone for a procedure. Accordingly, a template guide model can be used to generate a patient-specific guide model. The patient-specific guide model may be used in a surgical procedure to address, correct, or mitigate effects of the identified deformity and may be used to generate a patient-specific guide that can be used in a surgical procedure for the patient.

Next, the method 300 may register 308 the preliminary guide model with one or more bones of the bone model. This step 308 facilitates customization and modification of the preliminary guide model to generate a patient-specific guide model from which a patient-specific guide can be generated. The registration step 308 may combine two models and/or patient imaging data and positions both models for use in one system and/or in one model.

Next, the method 300 may design 310 a patient-specific guide model based on the preliminary guide model. The design step 310 may be completely automated or may optionally permit a user to make changes to a preliminary guide model or partially completed patient-specific guide model before the patient-specific guide model is complete. A preliminary guide model and patient-specific guide model are two examples of an instrument model. As used herein, “instrument model” refers to a model, either physical or digital, that represents an instrument, tool, apparatus, or device. Examples of an instrument model can include a cutting guide model, a resection guide model, an alignment guide model, a reduction guide model, a patient-specific tendon trajectory guide model, a positioner model, a positioning guide model, and the like. In one embodiment, a patient-specific guide and a patient-specific guide model may be unique to a particular patient and that patient's anatomy and/or condition.

The method 300 may conclude by a step 312 in which patient-specific guide may be manufactured based on the patient-specific guide model. Various manufacturing tools, devices, systems, and/or techniques can be used to manufacture the patient-specific guide.

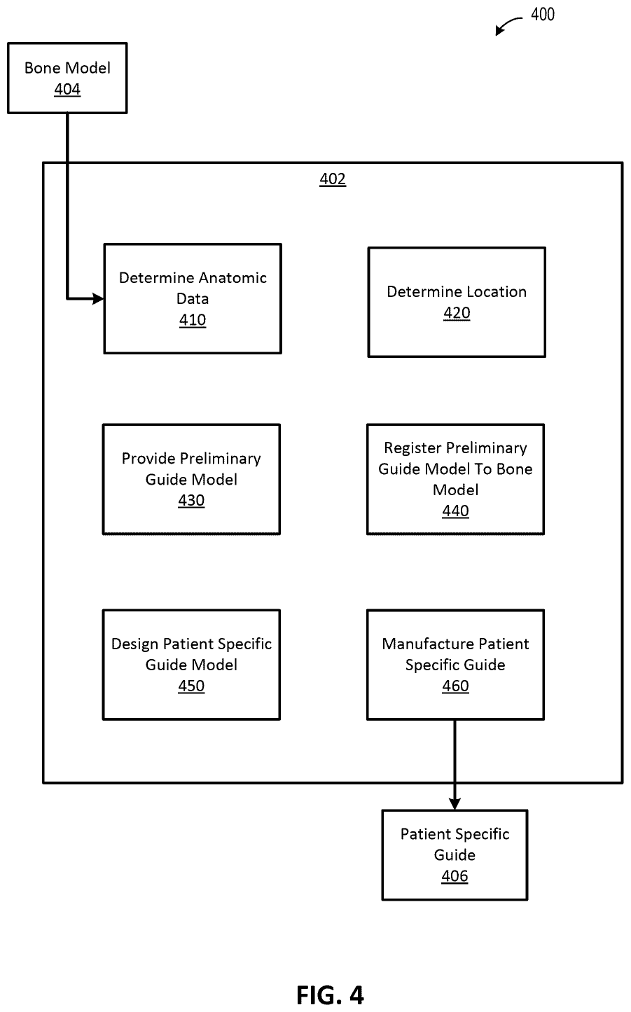

FIG. 4 illustrates an exemplary system 400 configured to generate one or more patient-specific instruments configured to facilitate surgical procedures, according to one embodiment. The system 400 may include an apparatus 402 configured to accept, review, receive or reference a bone model 404 and provide a patient-specific guide 406. In one embodiment, the apparatus 402 is a computing device. In another embodiment, the apparatus 402 may be a combination of computing devices and/or software components or a single software component such as a software application.

The apparatus 402 may include a determine anatomic data module 410, a determine location module 420, a provision module 430, a registration module 440, a design module 450, and a manufacturing module 460. Each of which may be implemented in one or more of software, hardware, or a combination of hardware and software.

The determine anatomic data module 410 determines anatomic data 412 from a bone model 404. In certain embodiments, the system 400 may not include a determine anatomic data module 410 if the anatomic data is available directly from the bone model 404. In certain embodiments, the anatomic data for a bone model 404 may include data that identifies each anatomic structure within the bone model 404 and attributes about the anatomic structure. For example, the anatomic data may include measurements of the length, width, height, and density of each bone in the bone model. Furthermore, the anatomic data may include position information that identifies where each structure, such as a bone is in the bone model 404 relative to other structures, including bones. The anatomic data may be in any suitable format and may be stored separately or together with data that defines the bone model 404.

In one embodiment, the determine anatomic data module 410 may use advanced computer analysis system such as image segmentation to determine the anatomic data. The determine anatomic data module 410 may determine anatomic data from one or more sources of medical imaging data, images, files, or the like. Alternatively, or in addition the determine anatomic data module 410 may use software and/or systems that implement one or more artificial intelligence methods (e.g., machine learning and/or neural networks) for deriving, determining, or extrapolating, anatomic data from medical imaging or the bone model. In one embodiment, the determine anatomic data module 410 may perform an anatomic mapping of the bone model 404 to determine each unique aspect of the intended osteotomy procedure and/or bone resection and/or bone translation. The anatomic mapping may be used to determine coordinates to be used for an osteotomy procedure, position and manner of resections to be performed either manually or automatically or using robotic surgical assistance, a width for bone cuts, an angle for bone cuts, a predetermined depth for bone cuts, dimensions and configurations for resection instruments such as saw blades, milling bit size and/or speed, saw blade depth markers, and/or instructions for automatic or robotic resection operations.

In one embodiment, the determine anatomic data module 410 may use advanced computer analysis system such as image segmentation to determine the anatomic data. The determine anatomic data module 410 may determine anatomic data from one or more sources of medical imaging data, images, files, or the like. The determine anatomic data module 410 may perform the image segmentation using 3D modeling systems and/or artificial intelligence (AI) segmentation tools. In certain embodiments, the determine anatomic data module 410 is configured to identify and classify portions of bone based on a condition of the bone, based on the bone condition. Such classifications may include identifying bone stability, bone density, bone structure, bone deformity, bone structure, bone structure integrity, and the like. Accordingly, the determine anatomic data module 410 may identify portions or sections or one or more bones based on a quality metric for the bone. Advantageously, that determine anatomic data module 410 can identify high quality bone having a viable structure, integrity, and/or density versus lower quality bone having a nonviable structure, integrity, and/or density and a plurality of bone quality levels in between.

Accordingly, the determine anatomic data module 410 can guide a surgeon to determine which areas of one or more bones of a patient are within a “soft tissue envelope” (bone of undesirable quality) as that bone relates to a particular deformity or pathology. Identifying the quality of one or more bones of the patient can aid a surgeon in determining what type of correction or adjustment is needed. For example, an ulceration that occurs due to a boney deformity can be mapped using the determine anatomic data module 410 in a way that a correction can be performed to correct the deformity and reduce pressure to an area and address the structures that were causing the pressure ulceration/skin breakdown.

In addition, the determine anatomic data module 410 and/or another component of the apparatus 402 can be used to perform anatomic mapping which may include advanced medical imaging, such as the use of CT scan, ultrasound, MRI, and bone density scans can be combined to effectively create an anatomic map that determines the structural integrity of the underlying bone.

Identifying the structural integrity of the underlying bone can help in determining where bone resections can be performed to preserve the densest bone in relation to conditions such as Charcot neuropathic, arthropathy where lesser dense bone can fail and collapse. It is well documented in the literature that failure to address and remove such lesser dense bone can ultimately lead to failure of a reconstruction and associated hardware.

The present disclosure provides, by way of at least the exemplary system 400, an anatomic map that can be part of anatomic data. The anatomic map can combine structural, deformity, and bone density information and can be utilized to determine the effective density of bone and help to determine where bone should be resected in order to remove the lesser dense bone while maintaining more viable bone to aid in the planning of the osteotomy/bone resection placement.

The determine location module 420 determines or identifies one or more recommended locations and/or trajectory angles for deployment of an instrument, graft, and/or soft tissue based on the anatomic data 412 and/or the bone model 404. In one embodiment, the determine location module 420 may compare the anatomic data 412 to a general model that is representative of most patient's anatomies and may be free from deformities or anomalies. The determine location module 420 can operate autonomously and/or may facilitate input and/or revisions from a user. The determine location module 420 may be completely automated, partially automated, or completely manual. A user may control how automated or manual the determining of the location and/or trajectory angles is.

The provision module 430 is configured to provide a preliminary guide model 438. The provision module 430 may use a variety of methods to provide the preliminary guide model. In one embodiment, the provision module 430 may generate a preliminary guide model. In the same, or an alternative embodiment, the provision module 430 may select a template guide model for a surgical procedure configured to enable locating the position for one or more instruments and/or providing a trajectory provided by the determine location module 420. In one embodiment, the provision module 430 may select a template guide model from a set of template guide models (e.g., a library, set, or repository of template guide models).

The registration module 440 registers the preliminary guide model with one or more bones or other anatomical structures of the bone model 404. As explained above, registration is a process of combining medical imaging data, patient imaging data, and/or one or more models such that the preliminary guide model can be used with the bone model 404.

The design module 450 designs a patient-specific guide (or patient-specific guide model) based on the preliminary guide model. The design operation of the design module 450 may be completely automated, partially automated, or completely manual. A user may control how automated or manual the designing of the patient-specific guide (or patient-specific guide model) is.

The manufacturing module 460 may manufacture a patient-specific guide 406 using the preliminary guide model. The manufacturing module 460 may use a patient-specific guide model generated from the preliminary guide model. The manufacturing module 460 may provide the patient-specific guide model to one or more manufacturing tools and/or fabrication tool. The patient-specific guide model may be sent to the tools in any format such as an STL file or any other CAD modeling or CAM file or method for data exchange. In one embodiment, a user can adjust default parameters for the patient-specific guide such as types and/or thicknesses of materials, dimensions, and the like before the manufacturing module 460 provides the patient-specific guide model to a manufacturing tool.

Effective connection of the guide to one or more bones can ensure that surgical steps are performed in desired locations and/or with desired orientations and mitigate undesired surgical outcomes.

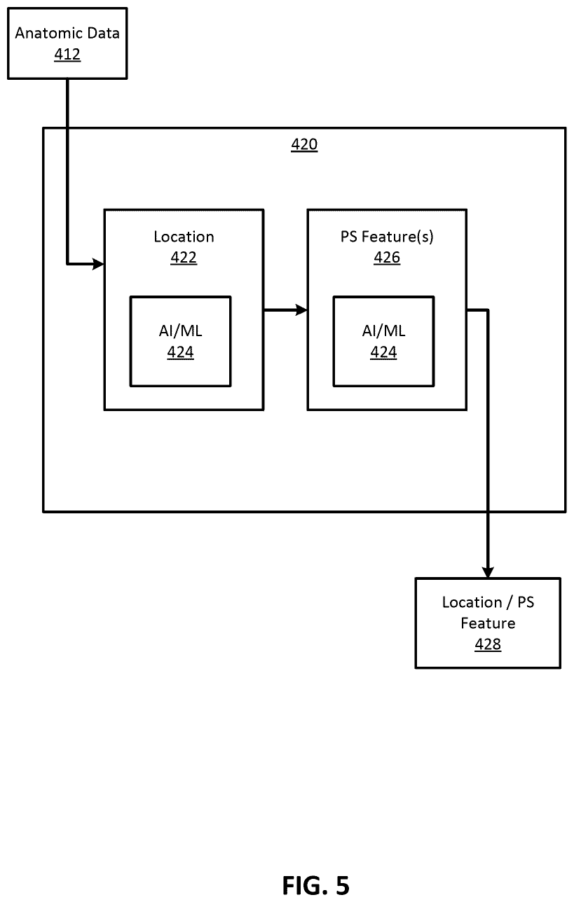

FIG. 5 illustrates an exemplary determine location module 420 configured to determine a recommended location and/or trajectory for steps and/or instruments in a surgical procedure, according to one embodiment. The determine location module 420 may factor in one or more landmarks on one or more surfaces of one or more bones of a patient of the bone model 404. The determine location module 420 may be completely automated, partially automated, or completely manual. A user may control how automated or manual the determination of the recommended location is. The user may provide instructions to the determine location module 420 to facilitate automatic or partially automated determination of one or more recommended locations.

The determine location module 420 may include a location module 422. The location module 422 may be configured for automated determination of a recommended location for steps and/or instruments in a surgical procedure. For example, in one embodiment, the location module 422 includes an artificial intelligence or machine learning module 424. The artificial intelligence or machine learning module 424 is configured to implement one or more of a variety of artificial intelligence modules that may be trained for identifying bones in the bone model 404, determining surfaces and/or sides of one or more bones, determining landmarks (both natural and/or abnormalities), determining axes of a bone, such as a longitudinal axis and/or a horizontal axis of a bone based on anatomic data 412 and/or a bone model 404. In another embodiment, the determine location module 420 may receive patient imaging data, a bone model, a CAD model or the like and use these inputs to determine a recommended location and/or trajectory in relation to one or more bones of a patient.

In one embodiment, the artificial intelligence or machine learning module 424 may be trained using a large data set of anatomic data 412 for healthy bones and a large data set of anatomic data 412 for bones with abnormalities and/or landmarks in which the abnormalities and/or landmarks have been previously identified and labeled in the dataset. The artificial intelligence or machine learning module 424 may implement, or use, a neural network configured according to the training such that as the artificial intelligence or machine learning module 424 accepts the anatomic data 412 for a particular patient, the artificial intelligence or machine learning module 424 is able to determine what one or more locations (e.g., a recommended location and one or more alternative locations for the guide.

The location module 422 may interact with a patient specific feature module 426. The patient specific feature module 426 may take one or more locations provided by the location module 422 and the bone model 404 and/or anatomic data 412 and determine suitable patient specific features. In certain embodiments, the patient specific features provided by the patient specific feature module 426 may include a number of resection features, an angle or trajectory for one or more resection features, a number, size, and/or position of bone attachment features, a number, size, or position of alignment guides or a combination of these. In certain embodiments, the patient specific feature module 426 may focus on resection features.

As with the determine location module 420, the patient specific feature module 426 may be completely automated, partially automated, or completely manual. A user may control how automated or manual the determination of the trajectory is. The user may provide instructions to the patient specific feature module 426 to facilitate automatic or partially automated determination of one or more trajectories. In one embodiment, the location module 422 includes an artificial intelligence or machine learning module 424 that facilitates determining one or more trajectories.

The determine location module 420 outputs a location/patient specific feature 428 for an orthopedic surgical procedure.

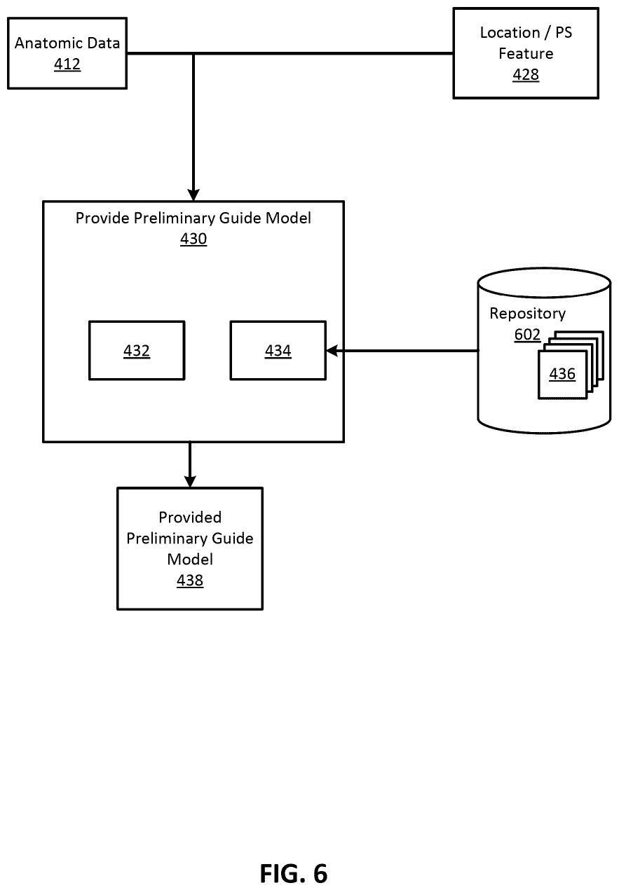

FIG. 6 illustrates an exemplary provision module 430 configured to provide a preliminary guide model, according to one embodiment. The provision module 430 may accept anatomic data 412 and a location/patient specific feature 428. In the illustrated embodiment, the provision module 430 may generate a preliminary guide model 438 (e.g., generate from ‘scratch’) or the provision module 430 may select a template guide model 436 automatically from a set of template guide models 436 stored in a repository 602. The provision module 430 may incorporate a variety of parameters in order to provision, generate, determine, or select a template guide model 436. For example, in addition to the anatomic data 412, the provision module 430 may include patient imaging data, deformity parameters for a variety of angular deformities (in all 3 planes) of the midfoot or hind foot and ankle, patient preferences, and/or surgeon input parameters.

In one embodiment, the provision module 430 may include a generator 432 and/or a selection module 434. In one embodiment, the generator 432 is configured to generate a preliminary guide model 438. In certain embodiments, the generator 432 may generate or create the preliminary guide model based on anatomic data and/or a bone model or a combination of these and no other inputs. (e.g. no model or predesigned structure, template, or prototype). Alternatively, or in addition, the generator 432 may generate or create the preliminary guide model using a standard set of features or components that can be combined to form the preliminary guide model. The generated preliminary guide model may subsequently be modified or revised by an automated process, and/or manual process, to generate the preliminary guide model used in this disclosure.

The selection module 434 may be configured to select a template guide model 436 for an osteotomy procedure configured to correct the deformity identified by the determine location module 420. In one embodiment, the provision module 430 may select a template guide model 436 from a set of template guide models 436 (e.g., a library, set, or repository of template guide models 436). In one embodiment, the template guide model 436 may include digital models. In another embodiment, the template guide model 436 may include physical models. In such an embodiment, the repository 602 may be a warehouse or other inventory repository. Where the template guide model 436 are physical models, the systems, modules, and methods of this disclosure can be used and the physical model may be milled or machined (e.g., a CNC machine) to form a patient-specific guide that conforms to the bone surfaces of the patient.

Selection of a suitable template guide model 436 may be completely automated and/or may be partially automated and/or may depend on confirmation from a user before a generated preliminary guide model or a proposed template guide model 436 becomes the preliminary guide model 438. In another embodiment, the selection module 434 may facilitate a manual selection by a user of a template guide model 436 that would become the preliminary guide model 438. The selection module 434 may use the anatomic data 412 or the bone model 404 or a combination of these to select a suitable template guide model that would become the preliminary guide model 438.

In another embodiment, the generator 432 may facilitate revisions or edits by a user of a generated guide model that will become the preliminary guide model 438. The selection module 434 may use the anatomic data 412 or the bone model 404 or a combination of these to select a suitable template guide model that would become the preliminary guide model 438.

The repository 602 may include any number of, and/or a variety of template guide models 436. The template guide models 436 may be distinguished based on a gender or age of the patient, which joint of a midfoot, hind foot, or ankle will be cut, which material will be used for the template guide, and the like. The template guide model 436 may differ from each other in what degree of deformity correction the template guide model 436 is designed to provide. In addition, the template guide models 436 may be distinguished based on how one or more features of the template guide model 436 are positioned, arranged, and/or configured relative to each other. For example, in certain template guide models 436, the number, position, and/or configuration of alignment features and/or bone attachment features (e.g., holes) may vary based on needs or preferences of patients, the nature of the deformity, and/or surgeon preferences.

In certain embodiments, the template guide models 436 may vary in how the slots (e.g., resection features) for the cuts are positioned, angled, and oriented relative to each other and/or to a longitudinal axis of respective bones at a joint for use with the template guide model 436. For example, in one template guide model 436 the slot 1352 for a resection of a metatarsal bone may be perpendicular to a longitudinal axis of the metatarsal bone and the slot 1350 may be angled relative to a longitudinal axis of the resection or cuboid bone such that once the two bones are brought together the deformity is corrected. Alternatively, in another template guide model 436 the slot for a resection of a metatarsal bone may be angled relative to a longitudinal axis of the metatarsal bone and the slot 1350 may be perpendicular to a longitudinal axis of the cuneiform or cuboid bone such that once the two bones are brought together the deformity is corrected.

The selection module 434 may be configured to automatically select a template guide model 436 and/or provide an automatic template guide model 436 recommendation that can be changed by a user, such as a surgeon. For example, in one embodiment, the provision module 430 and/or selection module 434 includes an artificial intelligence or machine learning module. The artificial intelligence or machine learning module is configured to implement one or more of a variety of artificial intelligence modules that may be trained for selecting a template guide model 436 based on anatomic data 412 and/or other input parameters. In one embodiment, the artificial intelligence or machine learning module may be trained using a large data set of anatomic data 412 for suitable template guide models 436 identified and labeled in the dataset by professionals for use to treat a particular deformity. The artificial intelligence or machine learning module may implement, or use, a neural network configured according to the training such that the artificial intelligence or machine learning module is able to select a suitable template guide model 436. The template guide model 436 selected by the selection module 434 can become the preliminary guide model 438.

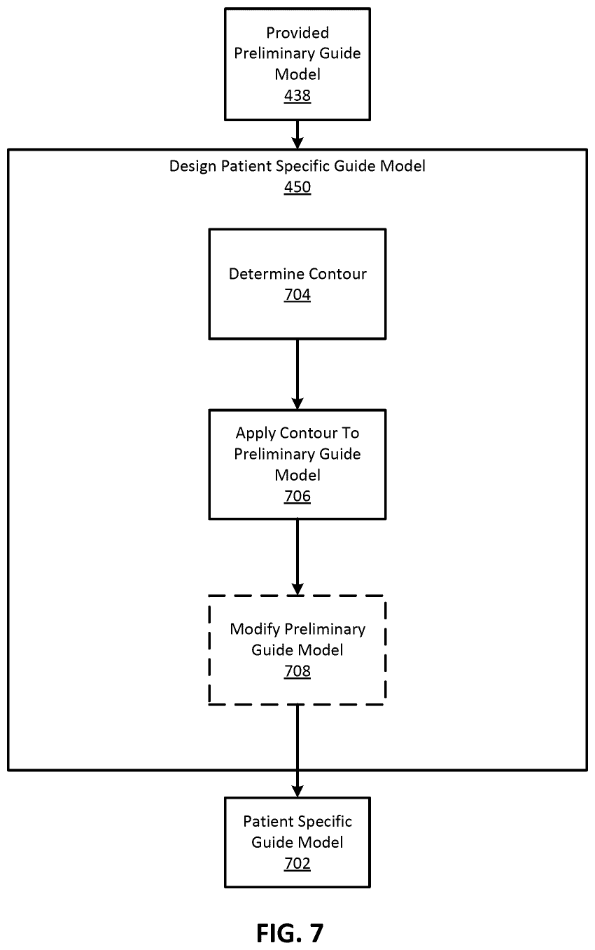

FIG. 7 illustrates an exemplary design module 450 configured to design a patient-specific guide model, according to one embodiment. The design module 450 may accept a preliminary guide model 438 and generate a patient-specific guide model 702. In one embodiment, the design module 450 includes a contour module 704, an application module 706, and/or an optional modification module 708.

Referring now to FIG. 7, the design module 450 may modify the preliminary guide model 438 such that the bone-facing and/or bone-contacting surfaces of the preliminary guide model 438 match a contour of the surfaces and/or joint of one or more bones where a step of an orthopedic procedure will be performed using the preliminary guide model 438. Said another way, the design module 450 may modify the preliminary guide model 438 to provide a bone engagement member on the preliminary guide model 438.

The contour module 704 may determine a contour of the bones that will contact the preliminary guide model 438. The contour module 704 may use a bone model 404 and/or anatomic data 412 to determine the contour. For example, the contour module 704 may determine the shape of a dorsal surface of a calcaneus 222.

The application module 706 may apply the contour to the provided preliminary guide model 438 to custom contour a bone engagement surface of the preliminary guide model 438 to match the shape, contour, and/or one or more landmarks of a bone, such as a dorsal surface of a calcaneus 222. Applying the contour to the preliminary guide model 438 may convert the preliminary guide model 438 to a patient-specific guide model 702.

Generation of the contours of bone engagement surface of the preliminary guide model 438 may be performed in various CAD programs. In some embodiments, the shapes of the corresponding surface dorsal surface of a calcaneus 222 may be obtained directly from the bone model 404, anatomic data 412, CAD models and/or CT scan data, and simply copied onto the preliminary guide model 438. Various operations may be used to copy surfaces from one object to another. Additionally, or alternatively, various Boolean operations, such as a Boolean subtraction operation, may be used to remove material from a model for the body of the preliminary guide model 438 with a shape that matches the dorsal surface of a calcaneus 222.

In certain embodiments, the design module 450 may include an optional module, such as a modification module 708. The modification module 708 may enable a user such as a technician or surgeon to make additional modifications to the design and configuration of the preliminary guide model 438. In one embodiment, the user can change any of the features, trajectories, fixation holes, handle engagement holes, angles, configurations, or parameters of the preliminary guide model 438. For example, a surgeon may be aware of other concerns or anatomic aspects of a patient, for example on an opposite foot or in connection with a hip or other orthopedic joint which motivate the surgeon to adjust an angle of one of more trajectories of the preliminary guide model 438.

Alternatively, or in addition, a user may use the modification module 708 to modify instrumentation such as a guide. The user may add, remove, or modify steps and/or the instrumentation to create a patient-specific surgical procedure. In this manner, a user may configure features of a preliminary guide model 438 or modified preliminary guide model to a patient-specific osteotomy procedure the surgeon is planning for the patient.

The user may review the preliminary guide model 438 and make adjustments or revisions or make no adjustments or revisions. The output of the modification module 708 and/or the application module 706 is a patient-specific guide model 702.

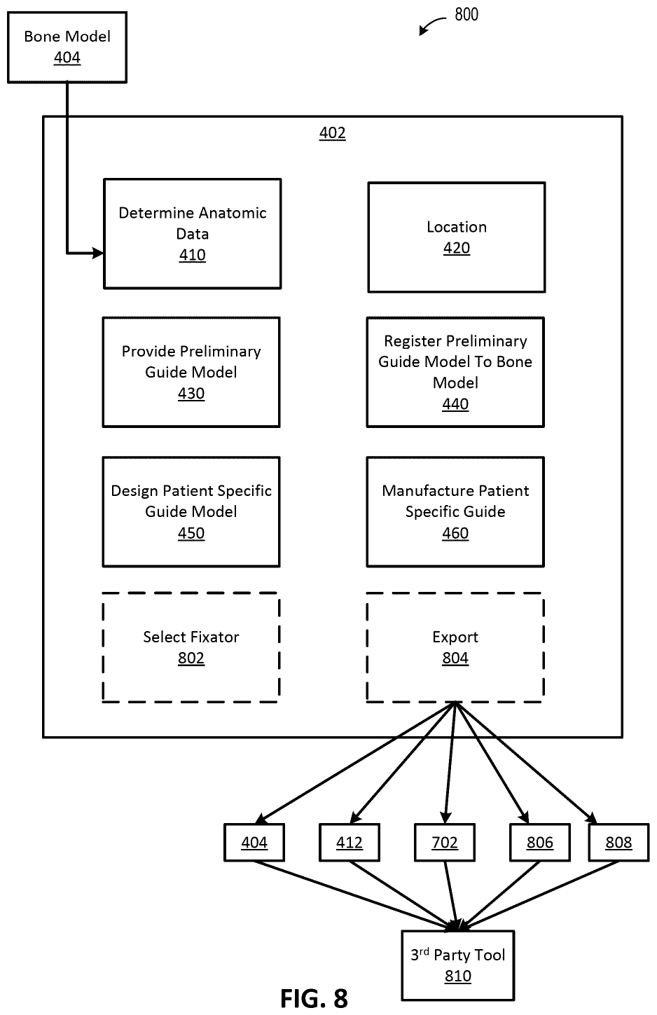

FIG. 8 illustrates an exemplary system 800 configured to generate one or more patient-specific instruments configured to correct a bone condition, according to one embodiment. The system 800 may include similar components or modules to those described in relation to FIG. 4. In addition, the system 800 may include a fixator selector 802 and/or an export module 804.

The fixator selector 802 enables a user to determine which fixator(s) to use for a surgical procedure planned for a patient. In one embodiment, the fixator selector 802 may recommend one or more fixators based on the bone model 404, the location, the trajectory, or input from a user or a history of prior surgical procedures performed. The fixator selector 802 may select a fixator model from a set of predefined fixator models or select a physical fixator from a set of fixators. The fixators may include a plate and associated accessories such as screws, anchors, and the like.

In one embodiment, the fixator selector 802 includes an artificial intelligence or machine learning module. The artificial intelligence or machine learning module is configured to implement one or more of a variety of artificial intelligence modules that may be trained for selecting fixator(s) based on anatomic data 412 and/or other input parameters. In one embodiment, the artificial intelligence or machine learning module may be trained using a large data set of anatomic data 412 for suitable fixator(s) identified and labeled in the dataset by professionals for use to treat a particular condition. The artificial intelligence or machine learning module may implement, or use, a neural network configured according to the training such that the artificial intelligence or machine learning module is able to select or recommend suitable fixator(s).

The export module 804 is configured to enable exporting of a patient-specific guide model 702 for a variety of purposes including, but not limited to, fabrication/manufacture of a patient-specific guide 406 and/or fixator(s), generation of a preoperative plan, generation of a physical bone model matching the bone model 404, and the like. In one embodiment, the export module 804 is configured to export the bone model 404, anatomic data 412, a patient-specific guide model 702, a preoperative plan 806, a fixator model 808, or the like. In this manner the custom instrumentation and/or procedural steps for a surgical procedure can be used in other tools. The preoperative plan 806 may include a set of step-by-step instructions or recommendation for a surgeon or other staff in performing a surgical procedure such as an osteotomy. The preoperative plan 806 may include images and text instructions and may include identification of instrumentation to be used for different steps of the surgical procedure. The instrumentation may include the patient-specific guide 406 and/or one or more fixators. In one embodiment, the export module 804 may provide a fixator model which can be used to fabricate a fixator for the surgical procedure.

The exports (404, 412, 702, 806, and 808) may be inputs for a variety of 3rd party tools 810 including a manufacturing tool, a simulation tool, a virtual reality tool, an augmented reality tool, an operative procedure simulation tool, a robotic assistance tool, and the like. A surgeon can then use these tools when performing a surgical procedure or for rehearsals and preparation for the surgical procedure. For example, a physical model of the bones, patient-specific guide 406, and/or fixators can be fabricated, and these can be used for a rehearsal operative procedure. Alternatively, a surgeon can use the bone model 404, preliminary guide model 438, and/or a fixator model to perform a simulated surgical procedure using an operative procedure simulation tool.

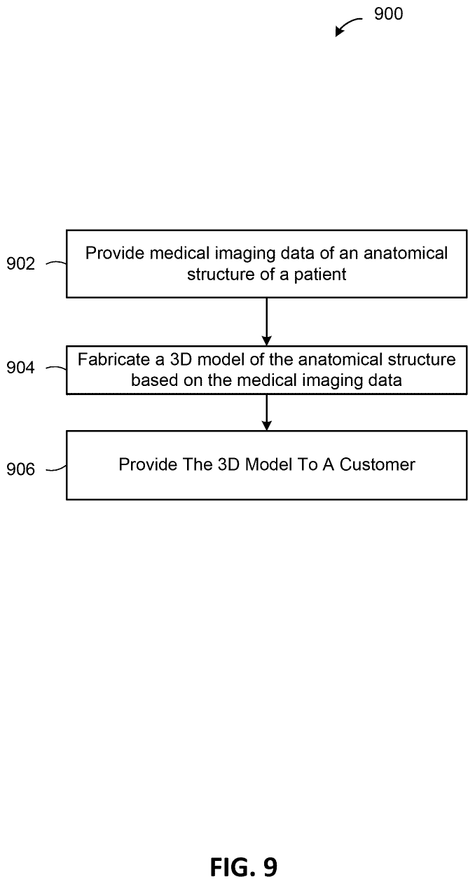

FIG. 9 illustrates a method 900 for providing a 3D physical bone model to a customer, according to one embodiment. FIG. 9 is a flowchart of an example process 900. In some implementations, one or more process blocks of FIG. 9 may be performed by a system such as, for example, system 800.

As shown in FIG. 9, process 900 may include providing medical imaging data of an anatomical structure of a patient (block 902). For example, system 800 may provide medical imaging data of an anatomical structure of a patient, in the form of a CT scan or other type of medical imaging. In another embodiment, the process 900 can include providing patient imaging data. The source of the medical imaging data and/or patient imaging data may be a medical image processing device, a medical image capture device, a storage device housing one or more digital files, or the like.

As also shown in FIG. 9, process 900 may include fabricating a 3D physical bone model of the anatomical structure based on the medical imaging data (block 904). For example, system 800 may fabricate a 3D physical bone model of the anatomical structure based on the medical imaging data or signal a third-party tool 810 to fabricate the 3D physical bone model. Advantageously, the 3D physical bone model is a physical representation of the anatomical structure. Alternatively, or in addition, the 3D physical bone model is a physical representation of a model of the anatomical structure.

In one embodiment, a system 800 and/or device may fabricate 904 a single 3D physical bone model. The single 3D physical bone model may comprise an assembly of a set of one or more 3D physical bone models of one or more anatomical structures. In certain embodiments, the fabricated 3D physical bone model is based on the medical imaging data of the patient. In this manner, the 3D physical bone model provides a surgeon with an accurate representation of the one or more anatomical structures. The surgeon has a tangible representation of the anatomy of the patient. The surgeon can examine the 3D physical bone model, hold the 3D physical bone model, rotate or reorient the 3D physical bone model, rehearse one or more steps of a surgical technique using the 3D physical bone model, manipulate the 3D physical bone model, combine the 3D physical bone model with an implant, engage the 3D physical bone model with one or more instruments, and perform a variety of other steps with the 3D physical bone model as part of preoperative, intraoperative, and/or postoperative steps in providing patient care.

Alternatively, or in addition, the system 800 and/or device may fabricate 904 a plurality of 3D physical bone models. The plurality of 3D physical bone models may each be distinct and separate from each other. The plurality of 3D physical bone models may then be coupled or connected to each or to some substrate and/or framework to form an assembly of 3D physical bone models, which whole assembly may be referred to as a 3D physical bone model.

The 3D physical bone model may be made of any material including a single material, a compound material, a mixture of materials, a plurality of materials, or the like. In one embodiment, one or more bone models of the 3D physical bone model may be made of varied materials that can be readily dissected and/or resected (e.g., rubber or silicon). For example, bone models of bones that are to be resected may be made from a pliable and/or readily dissected material such as rubber or silicon, while other bone models of the 3D physical bone model may be made from more rigid materials such as nylon or bone substitute materials.

In certain embodiments, the 3D physical bone model is made of a material suitable for use in sterilization equipment such as an autoclave. Because the 3D physical bone model can be readily sterilized, a surgeon can use, visualize, and/or interact with the 3D physical bone model in the operating room (e.g., during the surgical procedure).

One example material for one or more parts of the 3D physical bone model is a polymer known as Nylon 12. Nylon 12 may also be used as a more economical alternative to materials such as metal such as Titanium or stainless steel. In addition, in certain embodiments, the 3D physical bone model is made from a material that can be used in an additive manufacturing process and/or machine to “print” the 3D physical bone model.

The 3D physical bone model may include a single 3D physical bone model and/or may comprise a combination of two or more 3D physical bone models. Each 3D physical bone model may include a body. In certain embodiments, the body is solid. The body may be transparent, translucent, or opaque. Alternatively, or in addition, the body may include one or more openings that enable structures on an opposite side of the body to be seen.

As further shown in FIG. 9, process 900 may include providing the 3D physical bone model to a customer, such as a patient, a surgeon, a facility, or the like. In certain embodiments, this block 906 may include providing the 3D physical bone model to a surgeon, patient, or facility for a surgical procedure (block 906). For example, system 800 may prepare and/or package the 3D physical bone model for shipment to the user, customer, patient, surgeon, facility, and/or technician. In certain embodiments, the system 800 may prepare and send duplicate copies of the 3D physical bone model to one or more of a user, customer, patient, surgeon, facility, and/or technician.

Advantageously, because the patient is the source of the medical imaging data, the 3D physical bone model represents an anatomical structure(s) of a particular patient. In certain embodiments, this block 906 may include providing the 3D physical bone model to a surgeon, patient, or facility for reasons other than for a surgical procedure. For example, a patient, a facility, and/or an insurance provider may receive the 3D physical bone model and/or a duplicate of the 3D physical bone model for use in research, record keeping, an audit or review process, a surgical procedure pre-approval, a souvenir, or the like. In certain embodiments, the 3D physical bone model alone is provided. Alternatively, or in addition, the 3D physical bone model may be provided together with one or more other components, such as in a system that may include one or more implants, one or more instruments, a preoperative plan, or the like.

Process 900 may include additional implementations, such as any single implementation or any combination of implementations described below and/or in connection with one or more other processes described elsewhere herein. In a first implementation, the anatomical structure may include a structure selected from a group including a foot, an ankle, a knee, a lower extremity, an upper extremity, a hip, a hand, a wrist, an elbow, a shoulder, a neck, a head, a skull, and/or a combination of these.

In a second implementation, alone or in combination with the first implementation, the medical imaging data may include data from a computerized tomography (CT) scan of the anatomical structure of the patient. Of course, certain embodiments may use other forms of medical imaging. A third implementation, alone or in combination with the first and second implementation, process 900 may include fabricating an implant or instrument for use with the anatomical structure in a surgical procedure. In certain embodiments, the instrument and/or implant is a patient-specific instrument and/or patient-specific implant. In one embodiment, the fabricated instrument and/or implant may be based on medical imaging data and/or patient imaging data.

A fourth implementation, alone or in combination with one or more of the first through third implementations, process 900 may include generating a CAD model of the anatomical structure of the patient. In a fifth implementation, alone or in combination with one or more of the first through fourth implementations, 3D physical bone model may include two or more interconnected physical model bone models. A sixth implementation, alone or in combination with one or more of the first through fifth implementations, process 900 further includes fabricating at least one additional 3D physical bone model of the anatomical structure based on the medical imaging data, the at least one additional 3D physical bone model having two or more interconnected physical bone models interconnected according to a second configuration; and providing the at least one additional 3D physical bone model for the surgical procedure.

In a seventh implementation, alone or in combination with one or more of the first through sixth implementations, the anatomical structure may include a plurality of bones of the patient, and fabricating the 3D physical bone model further may include interconnecting two or more bone models of the 3D physical bone model to model interconnections of two or more bones of the plurality of bones of the patient.

Although FIG. 9 shows example blocks of process 900, in some implementations, process 900 may include additional blocks, fewer blocks, different blocks, or differently arranged blocks than those depicted in FIG. 9. Additionally, or alternatively, two or more of the blocks of process 900 may be performed in parallel.

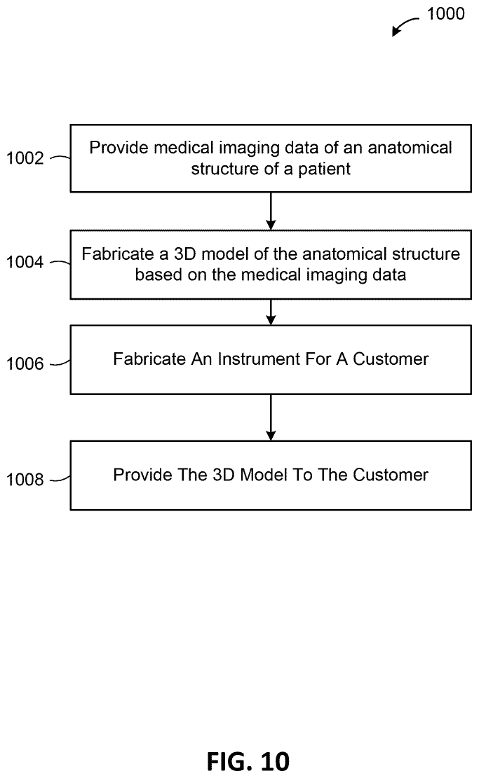

FIG. 10 illustrates a method 1000 for providing a 3D physical bone model to a customer, according to one embodiment. FIG. 10 is a flowchart of an example process 1000. In some implementations, one or more process blocks of FIG. 10 may be performed by a system such as, for example, system 800 or system 400.

Although FIG. 10 shows example blocks of process 1000, in some implementations, process 1000 may include the same blocks, additional blocks, fewer blocks, different blocks, or differently arranged blocks than those of example process 900 depicted in FIG. 9. For example, in one embodiment, block 1002, and block 1004 may be the same as corresponding block 902, and block 904. Block 1008 may be the same as corresponding block 906.

In addition to block 1002, block 1004, and block 1008, process 1000 may include fabricating 1006 an instrument for a customer, such as a patient, a surgeon, a facility, or the like (block 1006). In one embodiment, the instrument is an implant that can be used in a surgical procedure. In another embodiment, the instrument is a tool that can be used for a surgical procedure.

In certain embodiments, the instrument is fabricated based on medical imaging data and/or anatomic data 412. In certain embodiments, this may mean that the instrument is patient-specific. In other words, the instrument is a three-dimensional (3D) physical instrument that is based on and/or similar to and/or identical to a patient-specific instrument designed using embodiments of the present disclosure. In particular, the 3D instrument includes one or more patient-specific aspects. For example, angles for resection guides, pin holes, bone engagement members may be custom made to match anatomy of a particular patient. In one embodiment, the 3D physical instrument is a patient-specific instrument that is configured for use on deformed osseous anatomy of a patient in a surgical procedure.

Alternatively, or in addition, the instrument may be fabricated to satisfy a set of generic or accepted specifications for size, dimension, thickness, durability, material, and the like. The instrument may be used for a single use or may be reusable.

The instrument may be made from a variety of materials. The instrument can be fabricated of materials including, but not limited to, metal, plastic, nylon, nylon 12, ceramic, wood, fiberglass, acrylic, carbon, biocompatible materials, biodegradable materials or the like. An instrument may be formed of any biocompatible materials, including but not limited to biocompatible metals such as Titanium, Titanium alloys, stainless steel alloys, cobalt-chromium steel alloys, nickel-titanium alloys, shape memory alloys such as Nitinol, biocompatible ceramics, and biocompatible polymers such as Polyether ether ketone (PEEK) or a polylactide polymer (e.g. PLLA) and/or others.

As with other 3D physical bone models described herein, the instrument may be fabricated using additive manufacturing processes and/or equipment. Alternatively, or in addition, the instrument may be fabricated using subtractive manufacturing processes and/or equipment.

In another embodiment, the instrument is a 3D physical bone model of another instrument, such as an implant, a guide, a tool, or the like that is to be used in a surgical procedure. For example, suppose the instrument is a graft for use in a surgical procedure. The surgeon may plan to harvest the graft from tissue of the patient. With the process 1000, a surgeon can obtain a physical example of a graft that is to be harvested. The surgeon may then evaluate whether the planned graft will accomplish the goals of the surgical procedure or if revisions to the surgical procedure plan should be made.

In certain embodiments, block 1008 of the process 1000 may include providing an implant or an instrument to the customer. Alternatively, or in addition, the process 1000 may include providing an implant or an instrument for the surgical procedure. Alternatively, or in addition, the process 1000 may include an additional block (not shown) that includes providing an implant or an instrument to the customer and/or for the surgical procedure.

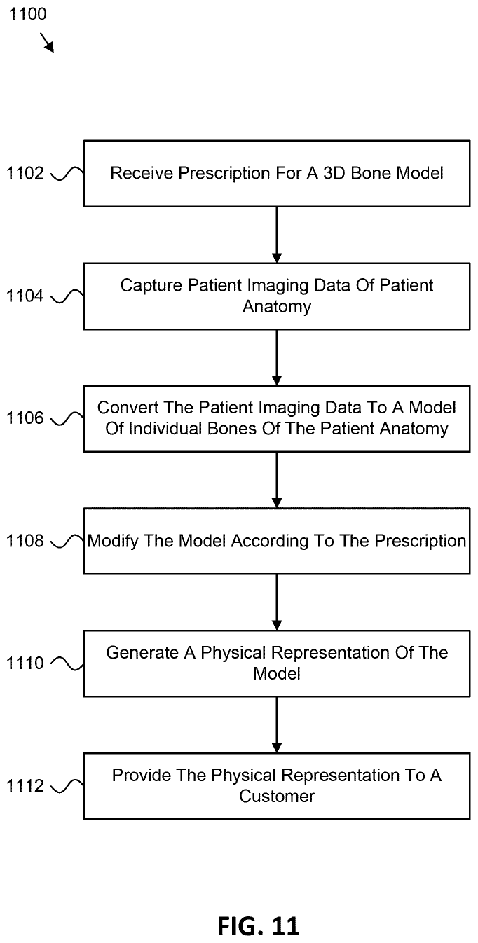

FIG. 11 illustrates a method 1100 for providing a 3D physical bone model to a customer, according to one embodiment. FIG. 11 is a flowchart of an example process 1100. In some implementations, one or more process blocks of FIG. 11 may be performed by a system such as, for example, system 800 or system 400.

Although FIG. 11 shows example blocks of process 1100, in some implementations, process 1100 may include the same blocks, additional blocks, fewer blocks, different blocks, or differently arranged blocks than those of example process 900, example process 1000, and/or example process 1200.

As shown in FIG. 11, process 1100 may include receiving a prescription for a 3D bone model (block 1102). For example, system 800, system 400, a digital computing device/system, or a manual system may receive a written physical prescription or a digital prescription from a doctor or surgeon for the 3D bone model. In certain embodiments, block 1102 may be required. In other embodiments, block 1102 may be optional.

As also shown in FIG. 11, process 1100 may include capturing patient imaging data of patient anatomy (block 1104). For example, a CT scanner may be used to collect CT images of a body part of a patient. In one embodiment, the CT scanner collects CT images of a patient's foot and ankle, hand and wrist, elbow, knee, hip, shoulder, spine, neck, head, or the like. Those of skill in the art will appreciate that other body parts of the patient can be used for the process 1100. Alternatively, block 1104 may not be needed as an alternate source for CT images of a body part of a patient (e.g., deformed osseous anatomy) may be used in the process 1100.

In certain embodiments, the process 1100 may include capturing patient imaging data of patient anatomy under different conditions or using different configurations and/or different positions. For example, CT images may be collected while a user places their weight on their ankle and/or foot. Alternatively, or in addition, CT images may be collected while a user dorsiflexes or plantarflexes or laterally rotates or medially rotates their ankle and/or foot. Those of skill in the art will appreciate that a variety of different conditions, different configurations, and/or various positions can be used when capturing patient imaging data of patient anatomy.

As also shown in FIG. 11, process 1100 may include converting the patient imaging data to a model of individual bones of the patient anatomy (block 1106). For example, in one embodiment, the process 1100 includes generating a computer model, such as a CAD model, of the patient anatomy (e.g., an anatomical structure). The computer model may be representative of the model of individual bones of the patient anatomy.

In certain embodiments, block 1106 may include segmenting patient imaging data in order to generate the computer model. For example, image segmentation tools, including image segmentation software, such as artificial intelligence can be used to examine patient imaging data and identify boundaries of individual bones within the patient imaging data. The position, size, orientation, and relationship of each individual bone to others in an image can be captured, preserved, and represented in a generated computer model. The computer model may comprise a single model that includes separate distinct bone models for each bone in the patient imaging data. Alternatively, or in addition, the computer model may comprise a plurality of models, each of separate distinct bones in the patient imaging data.

As also shown in FIG. 11, process 1100 may include modifying the model according to the prescription (block 1108). For example, in one embodiment, the patient anatomy or anatomical structure may include a deformed bone condition. In such an embodiment, block 1108 and/or the process 1100 may further include generating a modified computer model of the patient anatomy or anatomical structure. The modified computer model may be altered from the computer model of the patient anatomy or anatomical structure of the patient such that the modified computer model remediates the bone condition. Block 1108 and/or the process 1100 may further include fabricating a correction 3D physical bone model based on the modified computer model; and providing the correction 3D physical bone model to a customer. Alternatively, or in addition, the correction 3D physical bone model may be provided for the surgical procedure. “Correction 3D physical bone model” refers to a 3D physical bone model that has been designed, configured, engineered, and/or arranged to represent how a patient anatomy or an anatomical structure will be or will look after a surgical procedure implements a correction. With respect to orthopedic surgical procedures, a correction 3D physical bone model can represent one or more bones of a patient after corrective surgical procedures have been completed.

Thus, in certain embodiments, a customer may receive both a 3D physical bone model (representing patient anatomy before a surgical procedure, (e.g., preoperative 3D physical bone model)) and a correction 3D physical bone model (representing a plan for resulting patient anatomy after a surgical procedure (e.g., postoperative 3D physical bone model)). The 3D physical bone model may represent anatomy of a patient before a surgical procedure. This can be helpful to a surgeon because they can see and handle the 3D physical bone model and the correction 3D physical bone model and confirm that the preoperative plan for a surgical procedure will accomplish the desired results. Or the surgeon can determine that the correction 3D physical bone model provides insufficient correction or provides too much correction. Accordingly, the surgeon can request or make changes to the surgical procedure to refine the surgical procedure.

The 3D physical bone model and/or correction 3D physical bone model provides a tangible representation of the anatomical structure of the patient. The ability to view, rotate, hold and/or manipulate a 3D physical bone model and/or correction 3D physical bone model can greatly assist a surgeon in planning, preparing, and/or visualizing stages for a surgical procedure. Furthermore, the surgeon may identify potential challenges that may not have been noticed before having access to a 3D physical bone model and/or correction 3D physical bone model.

Alternatively, or in addition, where the process 1100 does not include receiving a prescription (block 1102), the process 1100 may include modifying 1108 the computer model according to criteria other than criteria that may originate from a prescription. For example, the computer model may be modified to include an indicator such as a label or other indicator to assist a surgeon in confirming that the surgical procedure is done on the desired anatomy (e.g., apply a marking of “Left” to the 3D physical bone model for a surgical procedure on a left foot of a patient). Alternatively, or in addition, the model may be modified based on an accepted standard procedure and/or standard of care.

In addition to the modifications discussed in other embodiments, in certain embodiments, the model may be modified 1108 to provide one or more structures and/or features that further facilitate the utility of the 3D physical bone model, a correction model, and/or one or more physical representations of these. As one example, the anatomical structure (e.g., patient anatomy) involved in the process 1100 may include a plurality of bones of a patient. Consequently, the computer model that represents bones of the patient may also include a plurality of independent bone models. In such an embodiment, the process 1100 and/or block 1108 may further include modifying the computer model such that two or more of the plurality of independent bone models are interconnected within the computer model. In certain embodiments, this may be accomplished by forming two or more interconnected structures that interconnect the two or more of the plurality of independent bone models of the computer model. Alternatively, or in addition, interconnects and/or other structures and/or fasteners can be added to the computer model to interconnect two or more independent bone models of the computer model.

By interconnecting bone models of the computer model, one or more 3D physical bone models generated using the process 1100 then include two or more interconnected structures representative of the two or more of the plurality of independent bone models of the computer model.

The interconnected structures can include any combination of bone models of the model. In one embodiment, the interconnected structures include the bone models of a first ray of a foot. In another embodiment, the interconnected structures include the bone models of a midfoot of a foot. In another embodiment, the interconnected structures include the bone models of a hind foot of a foot. In another embodiment, the interconnected structures include the bone models of all the bones of a foot of a particular patient. In certain embodiments, bone models may be connected using interconnects that are hidden or less visible such that a user has a clear view of joints, joint gaps between bones, and the like. Alternatively, or in addition, the interconnects may be visible and clearly noticeable to a user so that the existence and/or purpose of the interconnects is clear. Alternatively, or in addition, interconnects may connect adjacent bone models such as those that are part of one or more joints. Alternatively, or in addition, interconnects may connect any two bone models of a model.

In one embodiment, the one or more structures and/or features formed or added by modifying the model may include an interconnect, a connection formed between adjacent bones (bone models) of the model. In certain embodiments, a computing device or a user may interact with the computer model and form an interconnect between one or more bone models of the model. In one example, a computing device or a user may “draw” or form a solid structure between cortical surfaces of two adjacent bones. For example, the computing device or a user may form a solid structure between articular surfaces of two adjacent bone models, such as at a joint. Alternatively, or in addition, a computing device or a user may form any number of connections and/or interconnects between one or more parts of two or more bone models. The structure and/or interconnect may be represented in the model as being of the same material as the bone models. Alternatively, or in addition, when the model is fabricated a material for the interconnect may be selected to match the material used in fabrication of physical representations of the bone models.

In certain embodiments, a computing device may interconnect two or more bone models. Alternatively, or in addition, a computing devices, such as for example an artificial intelligence system, may operate alone and/or in combination with one or more users to provide an automated, semiautomated, and/or user assisted process step for interconnecting one or more parts of two or more bone models.

In certain embodiments, a computing device or a user may form a fastener (in model form), an interconnect, or other structure that joins two bone models. The fastener may be a temporary fastener or a permanent fastener. In one embodiment, the fastener is internal threads in a hole of one bone and a post or protrusion with corresponding external threads. Other examples of interconnects and/or fasteners include but are not limited to snaps, hook and loop, an adhesive, a tether and anchor, a spring anchored on one or both ends, a pliable tether (e.g., rubber band), a rod and socket, a ball and socket, an interface (such as a mechanical interface), or the like.

In one embodiment, the process 1100 includes at block 1108 providing interconnects in the form of an adjustable interconnect and/or an adjustable interface. These kinds of interconnects may be advantageous because when provided in a 3D physical bone model, adjustable interconnects may permit a surgeon to reposition one or more physical bone models of the 3D physical bone model into new positions and the one or more physical bone models may remain in the new position until repositioned.

In one embodiment, the one or more structures and/or features formed or added by modifying the model may include a cutaway or break away section of one or more bone models. For example, with a distal tibia, block 1108 may include forming a cut face between two bone fragments of a tibia, such as between a medial malleolus and a tibia and/or an interconnect that reduces and reconnects the two bone fragments. Advantageously, such a modification to the bone model can result in a physical representation of the tibia that can enable a surgeon to readily connect or disconnect the medial malleolus to expose internal bone surfaces such as a superior cortex of a talus.

In one embodiment, the one or more structures and/or features formed or added by modifying the model may include one or more cuts, channels, holes, bone tunnels, or other similar features formed in one or more bone models. For example, one or more channels may be formed in the bone models to represent planned resections. Alternatively, or in addition, channels may be formed to form a wedge that is separated from the bone of the bone model. These cuts, channels, holes, bone tunnels, may be formed based on best practices, standard operating procedures, a surgeon preferences, a preoperative plan, or the like. In certain embodiments, bone fragment of the bone model formed by cuts, channels, holes, bone tunnels, may be partially separated or wholly separated in different embodiments. Wholly separated bone model fragments may be completely separate or may include an interconnect to keep two or more bone fragments of a bone model in position.

Those of skill in the art will appreciate that a variety of other structures and/or features that can be formed or added by modifying the model at block 1108. Each of these other structures and/or features are within the scope of this present disclosure.

As also shown in FIG. 11, process 1100 may include generating a physical representation of the modified model (block 1110). For example, in one embodiment, a computing device and/or user may send instructions to a fabrication system, apparatus, or device (e.g., a fabricator) that instruct the fabricator to fabricate a physical representation of the modified model and/or of portions of the modified model (e.g., one or more bone models of the model). Those of skill in the art will appreciate a variety of different fabricators can be used and that these may be available onsite or offsite at a third party. Furthermore, the fabricator may fabricate the physical representation using additive manufacturing methods and/or subtractive manufacturing methods and/or molding manufacturing methods, or the like.

Advantageously, generating a physical representation of the modified model can include generating one or more interconnects and/or fasteners added to the model and/or modified model. Thus, one or more physical bone models of the 3D physical bone model can be interconnected to each other by way of interconnects.

In certain embodiments, block 1110 includes sending instructions to a 3D printer that uses an additive manufacturing process to fabricate a physical representation of a modified model using a medium such as polymer beads, sand, feed stock, cable, or the like. In one embodiment, the 3D printer prints the physical representation (also referred to as a 3D physical bone model) out of supply material called Nylon 12 in a bead, powder and/or sand form. Using a material like Nylon 12 is advantageous because it is economical, includes the relevant details, can be readily cleaned and prepared for use, can be sterilized for use in an operating room, etc.

In certain embodiments, the anatomical structure may be bones of a patient and may exclude soft tissue such as tendons, cartilage, ligaments, muscle, skin, or the like. Consequently, since the model positions and sizes and/or configures the bones to match the configuration, position, and alignment of the patient, certain anatomical structures such as bones can be separate and independent of any other structures in the model. Advantageously, a computing device and/or user may modify the model to include one or more interconnects that structurally join one or more bones to one or more other bones.

Since the 3D physical bone model is an accurate physical representation of the bone models of the model, the 3D physical bone model also includes two or more physical bone models joined by one or more interconnects. For example, where the model is a foot of a patient, the 3D physical bone model is an accurate physical representation of the bones of the foot and the model may include no models of soft tissue of the patient. The model may be modified to include an interconnect at each joint that corresponds to the actual joints of the bones of the patient. In one embodiment, the interconnect is solid material that connects one bone to another bone of the joint. Furthermore, in certain embodiment, the interconnect may be formed near a midpoint or center point between the two bones. In this manner, details of surface contours of each bone at the joint can be seen and/or used to fit or position one or more instruments or implants that are to be used in a surgical procedure.

In certain embodiments, one or more physical bone models of the 3D physical bone model may include at least one physical bone model having an indicator that identifies the at least one physical bone model for a user, such as a surgeon. Those of skill in the art will appreciate that the indicator can identify a variety of features and/or aspects the one or more physical bone models. For example, the 3D physical bone model may be 3D printed from a material such as Nylon 12, which may have a black color.

Alternatively, or in addition, one or more physical bone models of an assembly (e.g., the 3D physical bone model) may be made from a different material or may be colored (e.g., by painting or dying the fabrication material) to give the one or more physical bone models a distinct color from other physical bone models. For example, physical bone models involved in a corrective surgical procedure may be colored blue to indicate position, orientation, and/or condition prior to the corrective surgical procedure and one or more physical bone models may be colored pink to indicate position, orientation, and/or condition after the corrective surgical procedure. In this example, the indicator comprises color provided by the color of material used in fabrication. Those of skill in the art will appreciate that the indicator can be associated with a one or more physical bone models using other techniques such as painting.

Other indicators may include symbols, letters, words, or the like formed on a surface of one or more physical bone models of the 3D physical bone model.

Alternatively, or in addition, the indicator can indicate a variety of aspects. For example, the indicator can indicate which bones have a deformity, which are misaligned, which are misaligned by x degrees for an angle such as an intermetatarsal (IM) angle, where x is a number between 2 and 90 degrees.

In certain embodiments, the indicator is an indicator for where a surgeon plans to make one or more cuts in one or more bones of a patient. As described above, these indicators may be included separate from, or in addition to, features such as channels, cuts, holes, or other structures in, on, or associated with the one or more physical bone models. Thus, a surgeon can get a 3D physical bone model that shows where the cuts are to be made and/or one that shows where the cuts are to be made and includes the cuts so that the surgeon can see the results that are possible.

As described above, two or more of the physical bone models of the 3D physical bone model can be interconnected. In one embodiment, the two or more of the physical bone models can be interconnected according to a first configuration. The first configuration can be selected from the group comprising an anatomical position, a normal position, a weight bearing position, one stage of a walking position, one stage of a running position, one stage of a jumping position, one stage of a grasping position, a dorsiflexed position, a plantar flexed position, a laterally rotated position, a medially rotated position, a deformed condition, a corrected condition, a preoperative condition, and a postoperative condition. In certain embodiments, a surgeon may indicate in a prescription a desired configuration for one or more 3D physical bone models provided using the process 1100.

In certain embodiments, the interconnect used to couple one physical bone model to another may be rigid and maintain the configuration for the bones, when the 3D physical bone model is fabricated. In certain embodiments, the interconnect may be a coupler. In another embodiment, the interconnect may be flexible, semiflexible, pliable, elastic, and/or rigid. A rigid interconnect can be advantageous because the physical bone models retain the original configuration they had when fabricated. A flexible, semiflexible, pliable, adjustable, and/or elastic interconnect can be advantageous because a user such as a surgeon can change the configuration for the physical bone models from an original configuration to an alternative configuration to assist with the planning and/or performance of the surgical procedure.

In one embodiment, the process 1100 can include additional steps and/or block 1110 can include fabricating at least one additional 3D physical bone model of the anatomical structure based on the medical imaging data. The at least one additional 3D physical bone model may include two or more physical bone models interconnected according to a second configuration.

The process 1100 and/or block 1110 can further include providing the at least one additional 3D physical bone model for the surgical procedure. For example, a surgeon may request a 3D physical bone model of bones of a patient's foot that includes a deformity and a 3D physical bone model of bones of a patient's foot that includes bones repositioned and/or reduced to provide a correction that addresses the deformity. The second configuration may be selected from the group comprising an anatomical position, a normal position, a weight bearing position, one stage of a walking position, one stage of a running position, one stage of a jumping position, one stage of a grasping position, a dorsiflexed position, a plantar flexed position, a laterally rotated position, a medially rotated position, a deformed condition, a corrected condition, a preoperative condition, and a postoperative condition.

As also shown in FIG. 11, process 1100 may include providing a physical representation of the model to a customer (block 1112). For example, in one embodiment, a fabricator or other person or entity may send the physical representation of the model (e.g., 3D physical bone model(s)) or a plurality of physical representations of the model to a surgeon, to the surgeon's office, to a hospital or other facility, or to another customer. In certain embodiments, a surgeon, customer, hospital, or other facility may include the means for fabricating the physical representation of the model on site, in which case block 1110 may include sending instructions for the fabrication to the site that has the capability to generate the physical representation of the model.

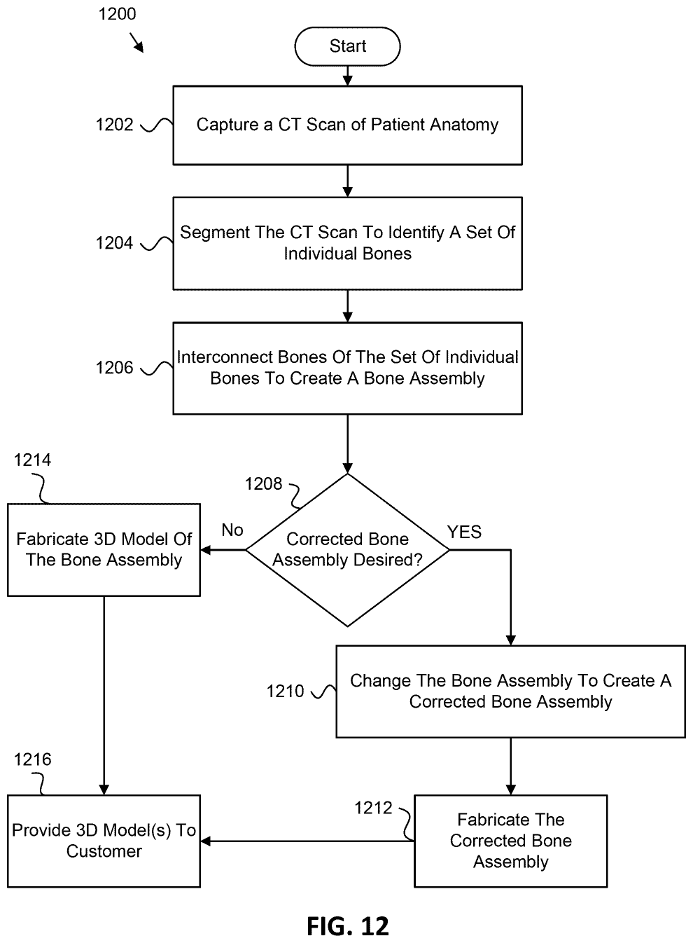

FIG. 12 illustrates a method for providing a 3D physical bone model to a customer, according to one embodiment. As shown in FIG. 12, a process 1200 may include capturing a CT scan of all or a portion of a patient's anatomy (block 1202). For example, a foot and ankle of a patient may be scanned by medical imaging equipment to generate a CT scan of one or more images of the structures of the patient's foot and/or ankle.

Although FIG. 12 shows example blocks of process 1200, in some implementations, process 1200 may include the same blocks, additional blocks, fewer blocks, different blocks, or differently arranged blocks than those of example process 900, example process 1000, and/or example process 1100.

As also shown in FIG. 12, process 1200 may include segmenting the CT scan to identify a set of individual bones of a patient (block 1204). Block 1204 which includes segmentation and/or image segmentation can be done by a programmed computing device, an artificial intelligence computer analysis system, machine learning, and/or automated system alone or together with an operator, technician, or the like.

As also shown in FIG. 12, process 1200 may include interconnecting one or more bones of the set of individual bones (in a model) to create a bone assembly (block 1206). A bone assembly or bone assembly model includes a collection of bones or bone models, such as a collection of bone models. In one embodiment, the bone models of the bone assembly may be interconnected to have a specific configuration. Block 1204 which includes interconnecting and/or coupling bones of the assembly can be done in whole or in part by a programmed computing device, an artificial intelligence computer analysis system, machine learning, and/or automated system alone or together with an operator, technician, or the like.

In certain embodiments, the process 1200 may include a determination 1208 regarding whether or not a corrected bone assembly is desired. A corrected bone assembly is one in which one or more bones of an assembly of bone models is adjusted, revised, repositioned, or modified such that the bones of the assembly form a corrected set of bones (e.g., modeled bones). Said another way, one or more bones of the assembly are in a desired position different from their original position(s).

If a determination 1208 is made to provide a corrected bone assembly, the process 1200 may continue to a next step in which a computing device and/or a user changes 1210 the bone assembly (or generates a new bone assembly) to create a corrected bone assembly. For example, if the patient has a hallux valgus condition, the bone assembly may model the bones of the patient and include the condition and the corrected bone assembly may model the bones of the patient with the condition remediated, removed, and/or corrected. The process 1200 may continue to step 1212 in which a user and/or computing device and/or system fabricates 1212 the corrected bone assembly. In certain embodiments, the user, a computing device, and/or a system fabricates 1212 a 3D physical bone model of the corrected bone assembly.

If a determination 1208 is made to not provide a corrected bone assembly, the process 1200 may continue to step 1214 in which a user and/or computing device and/or system fabricates 1214 a 3D physical bone model of the bone assembly. Next, the process 1200 ends in step 1216 in which one or more 3D physical bone models (e.g., a bone assembly and a corrected bone assembly) are provided 1216 to a customer.

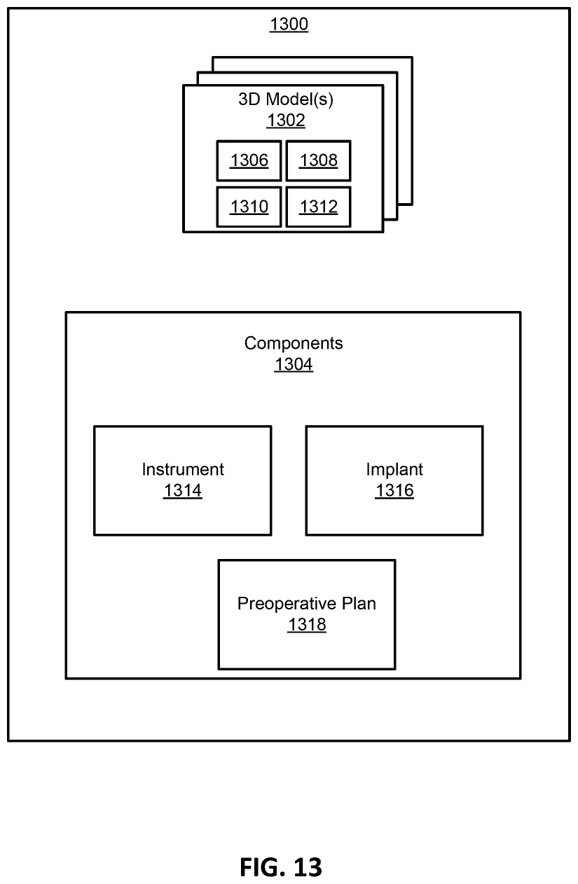

FIG. 13 illustrates an exemplary system 1300, according to one embodiment. The system 1300 can include a storage device and a processor executing program instructions stored in the storage device, the processor configured to: provide medical imaging data of an anatomical structure of a patient; fabricate a 3D physical bone model of the anatomical structure based on the medical imaging data; and provide the 3D physical bone model for the surgical procedure.

Alternatively, or in addition, the system 1300 can include one or more 3D physical bone models 1302 and one or more components 1304. As described herein, the 3D physical bone models 1302 can include a variety of features and/or attributes and/or aspects. For example, the 3D physical bone models 1302 can include sets of physical bone models coupled or interconnected according to one or more configurations 1306, may include one or more features 1308 such as cut channels, holes, or the like, may include one or more indicators 1310, and/or may include one or more interconnects 1312 of the same and/or different types.

The components 1304 include one or more items that can be used by a surgeon for a surgical procedure. For example, in one embodiment, the components 1304 can include one or more instruments 1314, one or more implants 1316, and/or one or more preoperative plans 1318. The one or more instruments 1314 may include positioners, aligners, resection guides, cutting tools, fasteners, compressors, distractors, rotation guides, targeting guides, and/or the like. The one or more implants 1316 may include a patient-specific implant, a generic implant, a sized implant, an implant to be sized intraoperatively, a graft such as a bone graft, and/or the like. The one or more preoperative plans 1318 may include a single plan with alternatives steps depending on how the patient presents. Alternatively, or in addition, the one or more preoperative plans 1318 can include a plurality of preoperative plans 1318 each prepared to handle certain situations that may arise prior to or during a surgical procedure. Of course, the one or more preoperative plans 1318 can be physical printed pages and/or digital formatted plans 1318 that can be presented on display devices to a surgeon preoperatively, intraoperatively, and/or postoperatively.

In certain embodiments, the system 1300 may be a kit that a surgeon can use before, after, or during a surgical procedure. For example, the kit 1300 can be sterilized for use in an operating room. Alternatively, or in addition, the kit 1300 can be sent to a surgeon for review, preoperative procedure practice, for sharing with the patient or be sent to a facility for inclusion in a set of instruments to be provided for a surgical procedure.

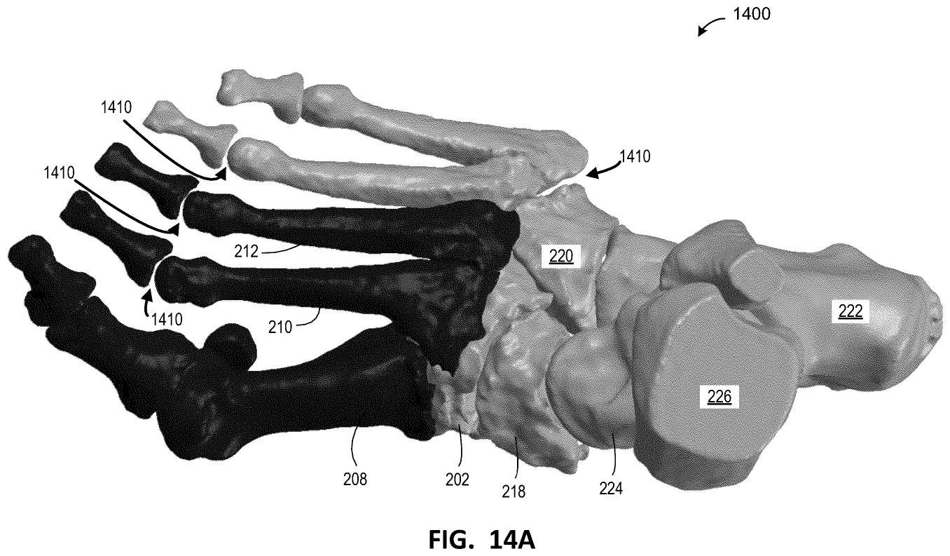

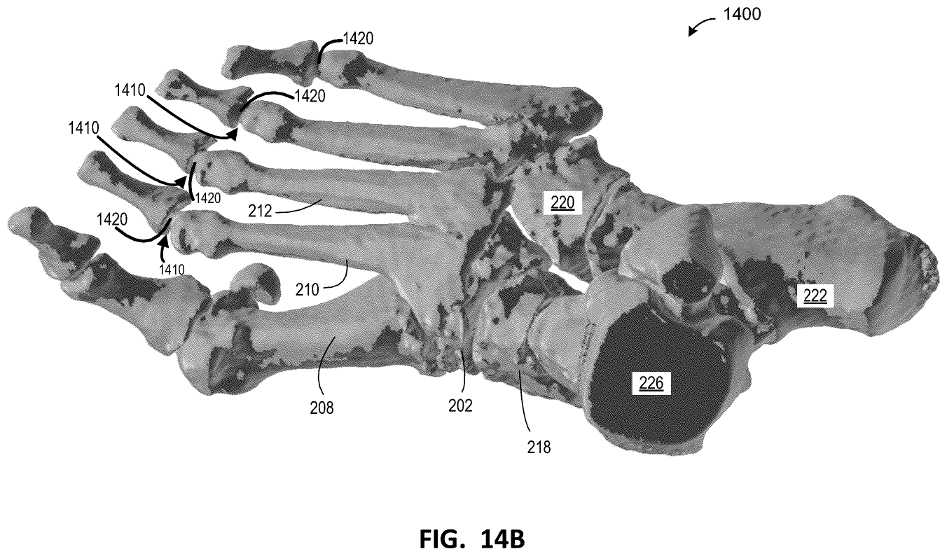

FIG. 14A is a superior to inferior view of a computer model 1400, according to one embodiment. The computer model 1400 is a representation of bones of a foot of a particular patient. The computer model 1400 includes a set of bone models, each bone model is a model of a particular bone of the foot. For example, the computer model 1400 includes a bone model of a medial cuneiform 202, first metatarsal 208, second metatarsal 210, navicular 218, cuboid 220, calcaneus 222, and a talus 224. Of course, other bone models of other bones of the foot are also shown in the computer model 1400 and/or FIG. 14A, not all the bones are labeled for brevity.

In certain embodiments, the bone models are solid model representations of the respective bones. Alternatively, or in addition, the bone models may comprise a surface that matches the same surface of a corresponding bone of the patient and have a hollow interior. In the illustrated embodiment, the bone models are solid models of the patient's bones. In certain embodiments, the bone models of the computer model 1400 may be of the same color and/or may be colored to match known or understood colors of patient anatomy that the bone models represent. Such coloring is one example of an indicator provided on the computer model 1400 which may be provided on a 3d physical bone model. In one embodiment, certain bone models may be colored to indicate a certain condition to a user. For example, blue colored bone models may represent one or more bones of a patient that is in a deformed condition. Bone models representing bones that are not in a deformed condition may be colored a neutral color such as brown, beige, grey, or white.

FIG. 14A includes a first metatarsal 208, second metatarsal 210, and/or third metatarsal 212 that are colored blue to represent that those bones and bones that would connect to them are in a deformed condition. Advantageously, the blue coloring readily identifies for a surgeon that these bones and or those connected to these bones have a deformed condition.

Those of skill in the art will appreciate that in one embodiment CT scans are segmented to identify bones of a patient within the scan including each bone's position and/or orientation in relation to one or more other bones included in the scan. Because the segmentation is focused on the bones of the patient, the soft tissue of the patient may not be segmented and/or included in the computer model 1400. Consequently, the bone models of the computer model 1400 are not connected to each other. As described above, a user, or an automated system, such as an artificial intelligence system, may add interconnects, fasteners, or other devices or features that connect the bone models of the computer model 1400.

FIG. 14A illustrates a computer model 1400 in which the bone models of the computer model 1400 have not yet been interconnected. This can be seen by the gaps shown in FIG. 14A between the bone models. Arrows 1410 indicate examples of gaps between bone models. Thus, if the computer model 1400 were to be printed, as is, for example by use of a 3D printer, the result would be a collection of physical bone models or 3D bone models that are not connected to each other. The present disclosure includes steps to interconnect or couple or join the bone models such that an interconnected 3D physical bone model results.

The computer model 1400 may be defined by data stored in a storage device, such as a memory, hard drive, SSD, or the like. The computer model 1400 may be viewable by using a computing device that presents the computer model 1400 in a graphical user interface, on a screen, in an augmented reality environment, in a virtual reality environment, or the like.

FIG. 14B is a superior to inferior view of the computer model 1400 after one or more bone models are interconnected, according to one embodiment. The same bone models of FIG. 14A are illustrated in FIG. 14B, however the gaps indicated by arrow 1410 are now filled by an interconnect 1420 that joins adjacent bone models. Those of skill in the art will appreciate that interconnects 1420 may be used between each bone model and an adjacent bone model or certain bone models may not be interconnected such that they can be free and separate for use by a surgeon to further examine and or utilize a 3D physical bone model that results from (generated from) the computer model 1400.

Alternatively, or in addition, different types of interconnects can be used between different bone models of the computer model 1400. For example, in one embodiment the interconnects 1420 are a solid structural connection between adjacent bones that can be fabricated from the same material used to fabricate physical bone models that represent the bone models. In another example, the interconnects 1420 can be some form of a fastener such as an adhesive, a screw and socket, a hook and loop, a snap, an elastic tether, a magnet and magnetic contact, a coupler such as a swivel joint, a ball and socket joint, and the like. In certain embodiments, an interconnect such as an adjustable interconnect may be configured to maintain a set position between two connected physical bone models until a user applies an external force to reposition one or more of the connected physical bone models. In this manner, a surgeon can reposition physical bone models and visualize trajectories and/or spacing and/or orientation of repositioned physical bone models in relation to each other and other anatomy without the physical bone models moving out of position.

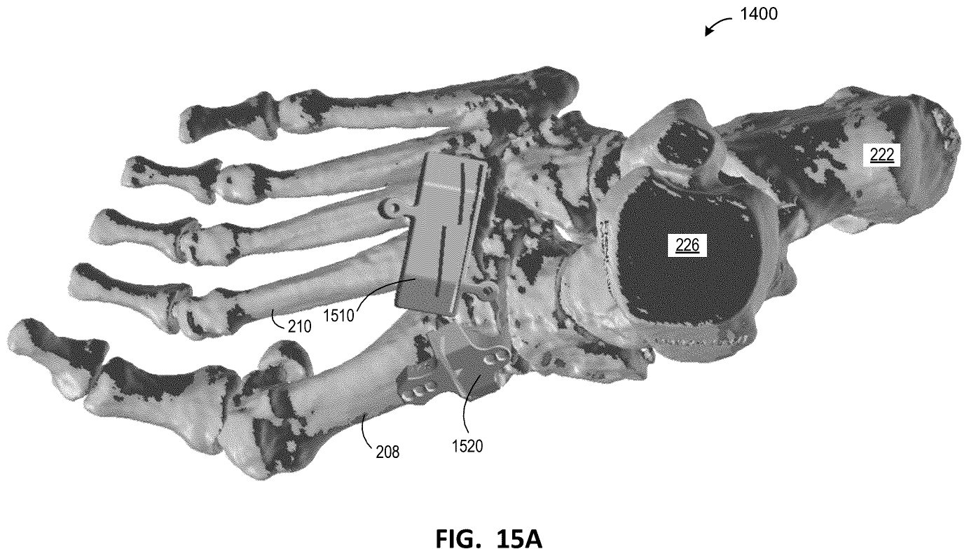

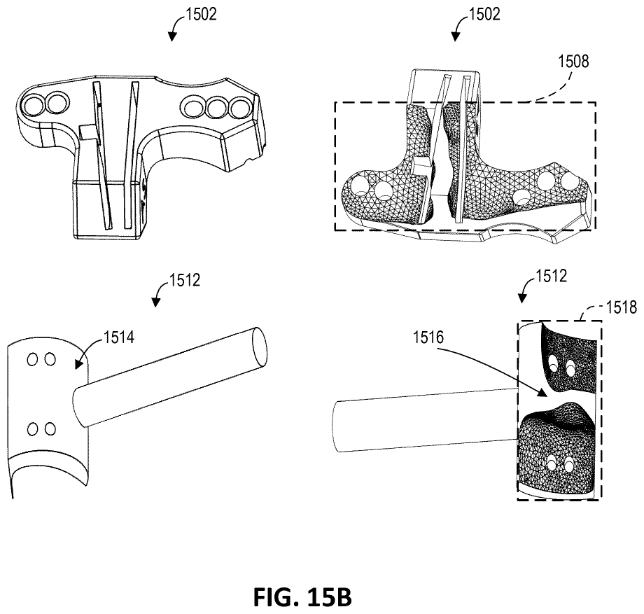

FIG. 15A is a superior to inferior view of the computer model 1400 that includes a couple of examples of model resection guides 1510, 1520. A first resection guide 1510 is a model of an instrument that can be used for a surgical procedure on the patient. In one example, the first resection guide 1510 can be used to address a metatarsus adductus condition. In one embodiment, the first resection guide 1510 is a patient-specific instrument. Another example resection guide 1520 can be used to perform a Lapidus procedure. The resection guide 1520 may be a patient-specific instrument. Advantageously, the model resection guides 1510, 1520 are positioned where a surgeon intends to use them on the patient in a procedure.

In one embodiment the model resection guides may be separate from the bone models. Thus, a surgeon may remove, replace, and/or reposition the first resection guide 1510 and/or resection guide 1520 as they review a 3D physical bone model of the computer model 1400. In this manner, a surgeon can rehearse how they can perform a surgical procedure using the first resection guide 1510 and/or resection guide 1520. Alternatively, or in addition, a surgeon can test or review how the first resection guide 1510 and/or resection guide 1520 interface with and/or engage one or more bones of the 3D physical bone model of the computer model 1400.

In one embodiment, one or the other of the first resection guide 1510 or resection guide 1520 may include a bone engagement member (e.g., a bone engagement surface, joint seeker, landmark registration feature, or the like) configured to engage with one or more bones and/or joints and/or other anatomical structures of the patient and the 3D physical bone model of the computer model 1400. Advantageously, a surgeon can practice engagement of a bone engagement member with one or more bones of the 3D physical bone model of the computer model 1400 prior to using the same first resection guide 1510 and/or resection guide 1520 in a surgical procedure on the patient.

Of course, the computer model 1400 can include a variety of other instruments with the computer model including but not limited to an alignment guide, a rotation guide, a reduction guide, a compression guide, a positioning guide, a fixation guide, one or more navigation guides, one or more implants, and the like. As with the resection guides, a surgeon can examine, place, position and/or rehearse with these other instruments in connection with a surgical procedure. Those of skill in the art will appreciate that the instruments provided with the computer model 1400 including example instruments first resection guide 1510 and/or resection guide 1520 are models of actual instruments that can be fabricated and that are configured and prepared for use in a surgical procedure. When fabricated the instruments may be made of a different material such as metal while the 3D physical bone model of the computer model 1400 may be made from a less expensive material such as a polymer.

Alternatively, or in addition, one or more model instruments may be connected to one or more bones that actual instruments will contact when used in a surgical procedure. In other words, certain model instruments may be positioned in predetermined/desired positions relative to one or more bones of the computer model 1400. This may be useful for a surgeon to see where actual instruments are to be used for the surgical procedure.

FIG. 15B illustrates exemplary physical instruments, according to one embodiment. As explained in connection with FIG. 15A, embodiments of the present disclosure can be used to fabricate both a three-dimensional (3D) physical model of patient osseous anatomy as well as one or more 3D physical instruments for use in a surgical procedure for a patient. FIG. 15B illustrates but a few examples of 3D physical instruments that can be provided using embodiments of the present disclosure.

FIG. 15B illustrates a non-bone-facing view of an example resection guide 1502, a bone-facing view of example resection guide 1502, a non-bone-facing view of an example positioning guide (e.g., positioner) 1512, and a bone-facing view of an example positioning guide (e.g., positioner) 1512.

Details of embodiments of the example resection guide 1502 can be found in at least U.S. patent application Ser. No. 17/020,630, entitled “PATIENT-SPECIFIC SURGICAL METHODS AND INSTRUMENTATION” filed Sep. 14, 2020, and U.S. patent application Ser. No. 17/681,674, entitled “PATIENT-SPECIFIC SURGICAL METHODS AND INSTRUMENTATION” filed Feb. 25, 2022, each of which is incorporated by reference herein in their entirety.

Details of embodiments of the example positioner 1512 can be found in at least U.S. patent application Ser. No. 18/439,454, entitled “APPARATUS, SYSTEM, AND METHOD FOR LAPIDUS CORRECTION” filed Feb. 12, 2024, and U.S. Provisional Patent Application No. 63/484,492, entitled “APPARATUS, SYSTEM, AND METHOD FOR LAPIDUS CORRECTION” filed Feb. 11, 2023, each of which is incorporated by reference herein in their entirety.

Advantageously, the example resection guide 1502 is a 3D physical instrument that is based on a patient-specific instrument designed using the embodiments of the present disclosure. In particular, a system such as system 400 can be used to fabricate a patient-specific guide 406. The patient-specific guide 406 may be based on a patient-specific guide model of the example resection guide 1502. The patient-specific instrument is configured for use on osseous anatomy of a particular patient for a surgical procedure. In certain embodiments, the patient-specific instrument is configured for use on deformed osseous anatomy of a particular patient for a surgical procedure. In certain embodiments, the 3D physical instrument (e.g., example resection guide 1502) is a physical embodiment of the patient-specific instrument. Therefore, the 3D physical instrument includes the attributes, aspects, and features of the patient-specific instrument.

Similarly, the example positioner 1512 is a 3D physical instrument that is based on a patient-specific instrument designed using the embodiments of the present disclosure. In particular, a system such as system 400 can be used to fabricate a patient-specific guide 406. The patient-specific guide 406 may be based on a patient-specific guide model of the example positioner 1512. The patient-specific instrument is configured for use on osseous anatomy of a particular patient for a surgical procedure. In certain embodiments, the patient-specific instrument is configured for use on deformed osseous anatomy of a particular patient for a surgical procedure. In certain embodiments, the 3D physical instrument (e.g., example positioner 1512) is a physical embodiment of the patient-specific instrument. Therefore, the 3D physical instrument includes the attributes, aspects, and features of the patient-specific instrument.

In the illustrated embodiment, the example resection guide 1502 is one instance of a 3D physical instrument and includes a non-bone-facing side 1504 and a bone-facing side 1506. In the illustrated embodiment, the non-bone-facing side 1504 is opposite the bone-facing side 1506. The bone-facing side 1506 includes a bone engagement member 1508. The bone engagement member 1508 is configured to engage with one or more bones of an osseous anatomy of a patient. Alternatively, or in addition, the bone engagement member 1508 is configured to engage with one or more bones of deformed osseous anatomy of a patient.

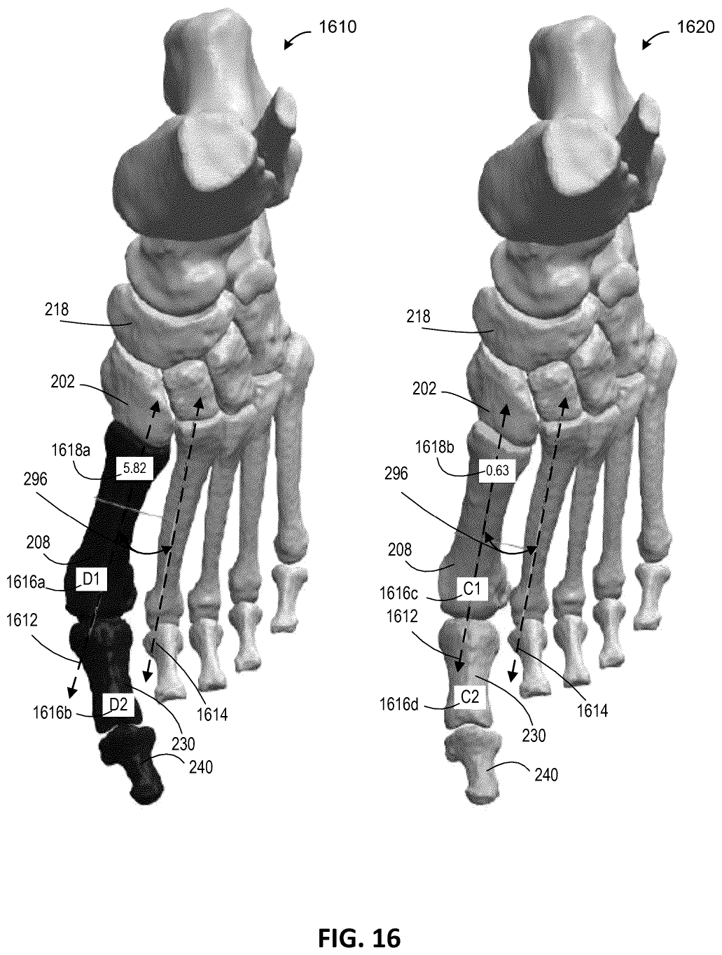

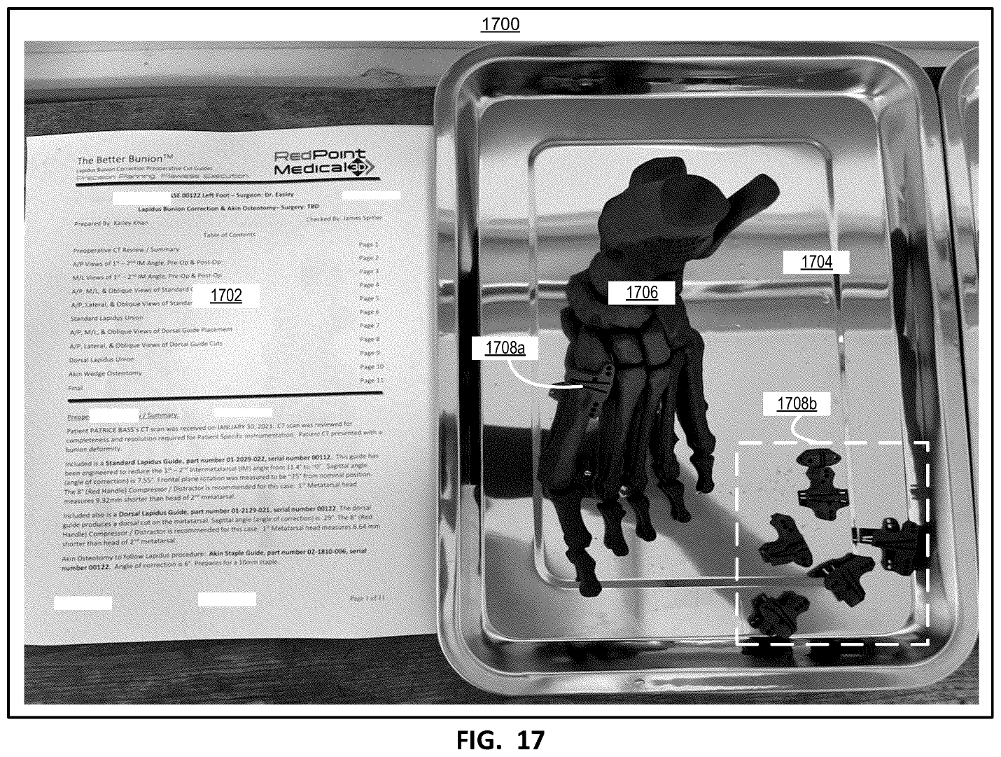

In addition, the bone engagement member 1508 is also configured to engage with one or more bones of a 3D physical bone model (See FIG. 16) corresponding to the osseous anatomy (or deformed osseous anatomy). Advantageously, in one embodiment, the bone engagement member 1508 engages a same first physical bone model of the 3D physical bone model that corresponds to a same first bone of the osseous anatomy (or deformed osseous anatomy). For example, a portion of a bone engagement member 1508 (e.g., a bone engagement surface) may engage with a medial cuneiform 202 physical bone model of the 3D physical bone model when the example resection guide 1502 is positioned in a desired and/or predetermined position on the 3D physical bone model. This same portion of a bone engagement member 1508 (e.g., a bone engagement surface) also engages with a medial cuneiform 202 physical bone of a patient when the example resection guide 1502 is positioned in a desired and/or predetermined position on the osseous anatomy (or deformed osseous anatomy). Of course, the example resection guide 1502 may include a plurality of bone engagement members 1508.

In the illustrated embodiment, the example positioner 1512 is one instance of a 3D physical instrument and includes a non-bone-facing side 1514 and a bone-facing side 1516. In the illustrated embodiment, the non-bone-facing side 1514 is opposite the bone-facing side 1516. The bone-facing side 1516 includes a bone engagement member 1518. The bone engagement member 1518 is configured to engage with one or more bones of an osseous anatomy of a patient. Alternatively, or in addition, the bone engagement member 1518 is configured to engage with one or more bones of deformed osseous anatomy of a patient.

In addition, the bone engagement member 1518 is also configured to engage with one or more bones of a 3D physical bone model (See FIG. 16) corresponding to the osseous anatomy (or deformed osseous anatomy). Advantageously, in one embodiment, the bone engagement member 1518 engages a same first physical bone model of the 3D physical bone model that corresponds to a same first bone of the osseous anatomy (or deformed osseous anatomy). For example, a portion of a bone engagement member 1518 (e.g., a bone engagement surface) may engage with a medial cuneiform 202 physical bone model of the 3D physical bone model when the example positioner 1512 is positioned in a desired and/or predetermined position on the 3D physical bone model. This same portion of a bone engagement member 1518 (e.g., a bone engagement surface) also engages with a medial cuneiform 202 physical bone of a patient when the example positioner 1512 is positioned in a desired and/or predetermined position on the osseous anatomy (or deformed osseous anatomy). Of course, the example positioner 1512 may include a plurality of bone engagement members 1518.

FIG. 16 illustrates exemplary 3D physical bone models, according to one embodiment. FIG. 16 illustrates 3D physical bone model 1610 and 3D physical bone model 1620. Advantageously, the 3D physical bone models are physical representations of the bone models of the computer model 1400.

The 3D physical bone model 1610 is a model that includes a plurality of physical bone models of a foot of the patient. The 3D physical bone model 1610 may include two or more physical bone models in a first configuration. The first configuration may be a deformed condition. Alternatively, or in addition, the first configuration matches or substantially matches a deformed osseous anatomy of a patient.

The 3D physical bone model 1620 is a model that includes a plurality of physical bone models of a foot of the same patient as 3D physical bone model 1610. The 3D physical bone model 1620 may include two or more physical bone models in a second configuration. The second configuration may be in a corrected condition.

In one embodiment, the first configuration may match or substantially match an osseous anatomy of a patient. In such an embodiment, the osseous anatomy may be a healthy normal anatomy of a patient. For example, a patient may be having a surgical procedure to shorten or lengthen one or more bones. In such an embodiment, the methods, systems, and/or apparatuses of the present disclosure can be used by the surgeon and/or the patient to facilitate the surgical procedure. The second configuration may model how the bones of a patient will be positioned and/or oriented after the surgical procedure (e.g., a bone lengthening or bone shortening procedure). The first configuration and/or second configuration can be selected from the group comprising an anatomical position, a normal position, a weight bearing position, one stage of a walking position, one stage of a running position, one stage of a jumping position, one stage of a grasping position, a dorsiflexed position, a plantar flexed position, a laterally rotated position, a medially rotated position, a deformed condition, a corrected condition, a preoperative condition, and a postoperative condition.

The 3D physical bone model 1610 represents bones in a deformed condition. Specifically, the 3D physical bone model 1610 accurately models a deformed osseous anatomy of a patient. For illustration, FIG. 16 includes a first ray longitudinal axis 1612 indicating where the longitudinal axis is for the first ray (e.g., first metatarsal 208 of 3D physical bone model 1610/1620) and a second ray longitudinal axis 1614 indicating where the longitudinal axis is for the second ray (e.g., first metatarsal 208 of 3D physical bone model 1610/1620). The angle between the first ray axis 1612 and the second ray axis 1614 is the IM angle 296. In the illustrated embodiment, in the 3D physical bone model 1610, the IM angle 296 is about 5.82 degrees and in the 3D physical bone model 1620, the IM angle 296 is about 0.63 degrees. As this example difference indicates, the 3D physical bone model 1620 is a corrected model and the 3D physical bone model 1610 is the deformed model.

In certain embodiments, an apparatus, system, and/or method according to the present disclosure includes providing an indicator on a 3D physical bone model 1610/1620. The indicator may convey to a surgeon, or other user, which bones are part of a deformed bone condition. In particular, the indicator can identify a physical bone model that corresponds to a patient bone that has a deformed bone condition. The patient bone may be a bone of an osseous anatomy and/or of a deformed osseous anatomy. Advantageously, the indicator is configured to convey information about one or more of the osseous anatomy, the patient, the surgical procedure, and the like. Thus, the indicator can be or communicate a patient ID, a patient name, a side of the body for the anatomy (e.g., left or right), what procedure the 3D physical bone model is intended for, and the like. Those of skill in the art will appreciate that a variety of different means, techniques, and/or mechanisms can be used to provide and/or include an indicator on, in, or in association with either or both of the 3D physical bone model 1610 and/or the 3D physical bone model 1620.

As an example, FIG. 16 illustrates two different types of indicators. FIG. 16 includes a bone indicator 1616 and an angle indicator 1618. In one embodiment, the bone indicator 1616 is an indicator that includes a mark or symbol that indicates what condition a physical bone model with the bone indicator 1616 is in.

In the illustrated embodiment, the bone indicator 1616a is a D1 symbol provided on a surface of a first bone model representing a first bone of a patient that is in a deformed condition (e.g., first metatarsal 208). Similarly, bone indicator 1616b is a D2 symbol provided on a surface of a second bone model representing a second bone of a patient that is in a deformed condition (e.g., proximal phalange 230). The D of the bone indicator 1616 may indicate that the associated bone model is in a deformed bone condition and the number may be a serial number that increases sequentially. The bone indicators 1616a, 1616b may be printed (embossed or debossed) or otherwise fabricated or affixed to a surface of a physical bone model of the 3D physical bone model 1610.

Similarly, in the illustrated embodiment, the bone indicator 1616c is a C1 symbol provided on a surface of a first bone model representing a first bone of a patient that is in a corrected condition or a repositioned condition (e.g., first metatarsal 208). Similarly, bone indicator 1616d is a D2 symbol provided on a surface of a second bone model representing a second bone of a patient that is in a corrected condition or a repositioned condition (e.g., proximal phalange 230). The C of the bone indicator 1616 may indicate that the associated bone model is in a corrected or repositioned condition and the number may be a serial number that increases sequentially. The bone indicators 1616c, 1616d may be printed (embossed or debossed) or otherwise fabricated or affixed to a surface of a physical bone model of the 3D physical bone model 1620.

In one embodiment, the angle indicator 1618 is an indicator that includes a mark or symbol that indicates an IM angle 296 between a first ray and a second ray of the 3D physical bone model 1610 and/or 3D physical bone model 1620. In the illustrated embodiment, the angle indicator 1618a is a numeric value indicating a number of degrees for the IM angle 196 provided on a surface of a physical bone model of the 3D physical bone model 1610 and the angle indicator 1618b is a numeric value provided on a surface of a physical bone model of the 3D physical bone model 1620. In this example, angle indicator 1618a is 5.82 degrees and angle indicator 1618b is 0.63 degrees. The angle indicator 1618a, 1618b, may be printed (embossed or debossed) or otherwise fabricated or affixed to a surface of a physical bone model of the 3D physical bone model 1610 or 3D physical bone model 1620.

The indicators provide a clear indication to a surgeon of information about the bones and/or other characteristics of the 3D physical bone model 1610 and/or 3D physical bone model 1620 and thus the osseous anatomy of a patient. In one embodiment, indicators may be implemented by way of coloring used for, on, or in the physical bone models of the 3D physical bone model 1610 and/or 3D physical bone model 1620. For example, in one embodiment, the color blue may be used to represent bones that are in a deformed condition. Alternatively, or in addition, in another example, the color pink may be used to represent bones that are in a corrected condition.

FIG. 17 illustrates an exemplary system 1700, according to one embodiment. In this example embodiment, the system 1700 may include a preoperative plan 1702 and/or a surgical tray 1704. The preoperative plan 1702 may be provided electronically in a digital format. Alternatively, or in addition, the preoperative plan 1702 may be printed and provided in a printed format. The preoperative plan 1702 may include recommendations, suggestions, images, measurements, medical imaging, information, features and/or the like that are specifically set up for a planned surgical procedure.

The surgical tray 1704 may include each of the components that are to be used in a planned surgical procedure. In one embodiment, the components of the surgical tray 1704 may be sterilized such that they can be brought into an operating room safely. In the example surgical tray 1704, the components include an example 3D physical bone model 1706 and one or more resection guides 1708. The 3D physical bone model 1706 may be in a predefined configuration, a configuration requested by the surgeon, and/or a deformed configuration.

In the illustrated embodiment, the 3D physical bone model 1706 is in a deformed configuration/condition. In the illustrated embodiment, the 3D physical bone model 1706 is fabricated from a polymer Nylon 12 having a black color. In certain embodiments, a plurality of resection guides 1708 may be provided, each resection guide 1708 may be a trial guide, intended to show a surgeon how the guide will fit on the bones of a patient. Using a trial guide a surgeon can see where cuts will be made using each respective resection guide 1708, when the resection guide 1708 is in a desired position.

A recommended resection guide 1708a may be positioned and/or connected to the 3D physical bone model 1706. Alternatively, or in addition, the recommended resection guide 1708a may be stamped with an indicator indicating that it is the recommended resection guide 1708a. Alternatively, or in addition, a plurality of alternative resection guides 1708b can be included in the surgical tray 1704. These alternative resection guides 1708b may provide different angles, different sizes of cuts, and other differences that a surgeon may want to consider either preoperatively or intraoperatively for the surgical procedure.

Of course, those of skill in the art will appreciate that the resection guides 1708 may not be trial resection guides 1708 and may instead be one or more resection guides 1708 intended for use in a surgical procedure. Consequently, the resection guides 1708 may be made of another material such as a metal such as a metal such as a stainless steel and/or titanium.

In the illustrated embodiment, the system 1700 may be a kit for use in an osteotomy procedure. The kit includes a first 3D physical bone model 1706 of deformed osseous anatomy of a patient for an osteotomy procedure. Advantageously, the first 3D physical bone model 1706 is a three-dimensional highly accurate model of physical bones of the patient. The kit also includes a preoperative plan 1702 for the osteotomy procedure. In certain embodiments, the preoperative plan 1702 is configured for use with a specific patient. The kit also includes a set of patient-specific instruments (e.g., resection guides 1708) for one or more stages of the osteotomy procedure.

In certain embodiments, the kit may also include a second 3D physical bone model. The second 3D physical bone model may include a plurality of physical bone models coupled to each other by way of a plurality of interconnects. In one embodiment, the plurality of physical bone models are coupled according to a predetermined configuration. Those of skill in the art will appreciate that a variety of predetermined configurations may be used. For example, the predetermined configuration may be selected from the group comprising an anatomical position, a normal position, a weight bearing position, one stage of a walking position, one stage of a running position, one stage of a jumping position, one stage of a grasping position, a dorsiflexed position, a plantar flexed position, a laterally rotated position, a medially rotated position, an eversion rotated position, an inversion rotated position, a deformed condition, a corrected condition, a preoperative condition, and a postoperative condition.

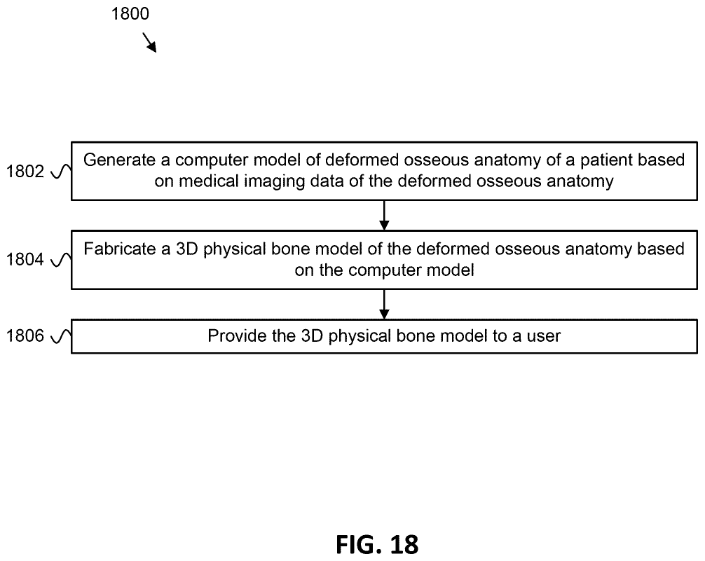

FIG. 18 is a flowchart of an example method 1800. In some implementations, one or more method steps of FIG. 18 may be performed by an apparatus, device, computing device, or system, such as, for example, system 800.

As shown in FIG. 18, method 1800 may include generating a computer model of deformed osseous anatomy of a patient based on medical imaging data of the deformed osseous anatomy (step 1802). Advantageously, the fidelity and accuracy of the medical imaging enables the computer model to accurately represent the deformed osseous anatomy. Alternatively, or in addition, the medical imaging can be used to generate anatomic data 412 that may then be used to form the computer model. In yet another alternative, the method 1800 may use anatomic data 412 to generate the computer model and the anatomic data 412 may come from one or more sources that may or may not include medical imaging data of a patient.

In certain embodiments, each of the bones of the deformed osseous anatomy may be modeled by way of separate virtual (e.g., computer generated and/or defined) bone models within the computer model. Together a plurality of virtual bone models may be organized into an assembly of bone models such that the bones of the computer model have the same, or substantially the same, size, shape, orientation, configuration, and/or positioning as corresponding bones of the deformed osseous anatomy. In certain embodiments, the computer model may not include soft tissue of the deformed osseous anatomy. Instead, where soft tissue joins or combines bones of the deformed osseous anatomy, the computer model may include gaps or spaces that may be connected using interconnects.

As also shown in FIG. 18, method 1800 may include fabricating a 3D physical bone model (e.g., 3D physical bone model 1610) of the deformed osseous anatomy based on the computer model (step 1804). In certain embodiments, the 3D physical bone model is configured to represent, or model, the physical attributes, characteristics, and/or aspects of the deformed osseous anatomy with a high degree of accuracy.

In certain embodiments, a user may provide instructions for the method 1800 such that certain bone models of the computer model have a greater degree of fidelity and/or accuracy in representing bones of a patient than others. In this manner, a user may efficiently use computing resources and/or fabrication resources to produce a suitable 3D physical bone model with less cost and in less time. As described herein, fabricating a 3D physical bone model may be done using additive manufacturing techniques, subtractive manufacturing techniques, molding manufacturing techniques, and/or the like.

As further shown in FIG. 18, method 1800 may include providing the 3D physical bone model to a user (step 1806). In one embodiment, providing the 3D physical bone model to a user may include preparing the 3D physical bone model for shipment and initiating a delivery to a user by way of a courier. Alternatively, or in addition, providing a 3D physical bone model to a user may include providing a user with credentials for accessing the 3D physical bone model from a fabrication system, facilitating, printer, or the like. It should be noted that the user may be a surgeon, a technician, a patient, or the like. Providing a 3D physical bone model to a patient can help assure the patient that the planned surgical procedure is in their best interests and may help calm their concerns. In particular, a patient may be able to visualize the planned correction and how the change can improve their health.

In certain embodiments, method 1800 step 1806 may not be included and may be an optional step. For example, where a user has means for fabricating the 3D physical bone model 1610 at a location where the user resides, works, or visits, the method 1800 may not include a step 1806. In such an embodiment, the user may implement steps 1802 and 1804 at their own desired location and then may access or use the 3D physical bone model 1610 as needed.

Advantageously, the 3D physical bone model (e.g., 3D physical bone model 1610) includes the details, aspects, characteristics, and/or features of the deformed osseous anatomy. This 3D physical bone model can assist a surgeon in planning and preparing for a surgical procedure. For example, the ability to hold, handle, review, and/or manipulate a 3D physical bone model of the deformed osseous anatomy may enable a surgeon to identify challenges that may arise in a surgical procedure in advance of the surgical procedure and provide the surgeon with time to prepare for and plan to how to handle those challenges. Furthermore, the ability to handle and review a 3D physical bone model having the deformed osseous anatomy enables a surgeon to visualize potential challenges for a surgical procedure and/or how a planned deformity correction may impact a quality of life of the patient and/or address needs of the patient.

Method 1800 may include additional implementations, such as any single implementation or any combination of implementations described below or above and/or in connection with one or more other methods or processes described elsewhere herein. In a first implementation, the deformed osseous anatomy may include a plurality of bones of the patient and fabricating a 3D physical bone model (e.g., 3D physical bone model 1610) may include coupling two physical bone models of the 3D physical bone model by way of an interconnect such that the two physical bone models connect in correspondence with patient bones of the deformed osseous anatomy.

For example, suppose the deformed osseous anatomy is a set of bones of a foot and ankle of a patient. The deformity may be a bunion condition affecting the first metatarsal 208, proximal phalange 230, second metatarsal 210, and third metatarsal 212. The surgical procedure may be a Lapidus procedure either separate and/or combined with an Akin procedure and/or correction for a metatarsus adductus (MTA).

As noted herein, in certain embodiments, soft tissue such as cartilage, tendons, ligaments, and/or skin may be omitted from the computer model generated using the method 1800 and/or the 3D physical bone model 1610. Thus, modeling individual physical bones of the deformed osseous anatomy by way of physical bone models of a 3D physical bone model 1610 may include coupling, joining, connecting, combining, and/or otherwise associating one physical bone model with another physical bone model by using an interconnect.

In a second implementation, alone or in combination with the first implementation, the interconnect may include an interface configured to enable a user to reposition one physical bone model in relation to another physical bone model coupled to the interface. In one embodiment, the interconnect is an interface between one physical bone model and another physical bone model. In certain embodiments, the interface may serve as an articulation interface. An articulation interface may enable one physical bone model to articulate in relation to an adjacent physical bone model. One example of an interface interconnect is a ball and socket. Another example is a hook and loop connector. Another example is a hinge. Another example is a swivel. In certain embodiments, the interconnect permits two coupled physical bone models to move freely with respect to each other. In another embodiment, the interconnect includes a friction fit or other resistance that maintains a position of two coupled physical bone models one positioned relative to each other.

Those of skill in the art will appreciate that the interconnect used can be one or more of a variety of different types, kinds, and/or technologies. From a simple fixed interconnect made of the same material as physical bone models of the 3D physical bone model 1610 to more complex interconnects such as mechanical hinges, living hinges, ball and socket interconnects, fasteners, springs, hook and look connectors, magnetic connectors, as well as the various examples provided herein. Further, those of skill in the art will appreciate that various permutations and/or combinations of interconnects may be used. For example, physical bone models of a 3D physical bone model 1610 that are included for context and/or completeness of the 3D physical bone model 1610 (those not directly involved in the deformity and/or the surgical procedure) such as those of an ankle and/or toes three through five may be coupled with a fixed interconnect or a rigid interconnect. In this manner, these bones of the 3D physical bone model 1610 can not be moved or repositioned but remain in the position as corresponding bones of the patient.

Alternatively, or in addition, other physical bone models of a 3D physical bone model 1610 directly involved in the deformity and/or the surgical procedure may be interconnected using interconnects that are pliable or removeable or detachable or that are adjustable such that one physical bone model can be repositioned relative to a coupled physical bone model. This distinction between physical bone models that are rigidly coupled and those that are free to be repositioned and/or removed can also serve as an indicator that communicates to a user which physical bone models are involved in the surgical procedure.

In certain embodiments, a separate interconnect may be used between a first physical bone model and a second physical bone model. The interconnect may be fabricated separately from the physical bone models. Alternatively, or in addition, the interconnect may be fabricated together with the physical bone models. In one embodiment, the interconnect may be generated and/or included in a computer model of the deformed osseous anatomy.

In an embodiment where the interconnect is separate from a first physical bone model and a second physical bone model, the method 1800 may include connecting or coupling the interconnect to a distal end of a first physical bone model and connecting or coupling the interconnect to a proximal end a second physical bone model. Advantageously, the method 1800 includes interconnecting one or more physical bone model of a 3D physical bone model 1610 such that the physical bone models have the same position, orientation, trajectory, and/or relationship to the other physical bone models of the 3D physical bone model 1610 as the bones of the deformed osseous anatomy of the patient.

In a third implementation, alone or in combination with the first and second implementation, the interconnect may include a rigid interconnect. A rigid interconnect can be useful for a user in maintaining a deformed bone condition within the 3D physical bone model 1610. Advantageously, a user is assured that even if the 3D physical bone model 1610 is handled the deformed osseous anatomy in the 3D physical bone model 1610 is maintained and can be referenced frequently without concern that the deformed osseous anatomy of the 3D physical bone model 1610 has been inadvertently changed. In certain embodiments, this advantage may be so important to a user, that the user prefers to have all the interconnects of the 3D physical bone model 1610 be rigid interconnects. Alternatively, or in addition, the user may desire and/or use one or more other embodiments of the method 1800 to generate a corrected model such as 3D physical bone model 1620. The interconnects of the 3D physical bone model 1620 may also be rigid interconnects to preserve the planned positions for the bones of the patient.

In a fourth implementation, alone or in combination with one or more of the first through third implementations, the interconnect may include a detachable interconnect. In certain embodiments, a user may request and/or the 3D physical bone model 1610 and/or 3D physical bone model 1620 may include detachable interconnects. The detachable interconnects can be helpful in rehearing for a surgical procedure, gaining access to other bones covered by bones, visualizing different repositioning plans or orientations, and the like. Examples of a detachable interconnect include, but are not limited to, a snap, a ball and socket, a hook and loop, an elastomer such as a rubber band, a snap hook, a spring hook, a snap-in connector, threaded fastener, quick-connect coupling, bayonet connector, a clevis fastener, other fasteners, and the like.

In a fifth implementation, alone or in combination with one or more of the first through fourth implementations, fabricating a 3D physical bone model may include coupling a plurality of physical bone models of the 3D physical bone model by way of a plurality of interconnects and where the plurality of interconnects may include rigid interconnects and adjustable interconnects. Certain interconnects may be adjustable and detachable. Other interconnects may be adjustable and not detachable. Examples of an adjustable interconnect include but are not limited to a friction fit ball and socket, a friction fit swivel connector, and the like.

In a sixth implementation, alone or in combination with one or more of the first through fifth implementations, fabricating a 3D physical bone model may include providing an indicator on or in a physical bone model of the 3D physical bone model, the indicator configured to convey information about at least one of the osseous anatomy, the patient, and the surgical procedure.

In a seventh implementation, alone or in combination with one or more of the first through sixth implementations, the indicator identifies a physical bone model corresponding to a patient bone having a deformed bone condition. In certain embodiments, the patient bone identified is a patient bone of the osseous anatomy.

In an eighth implementation, alone or in combination with one or more of the first through seventh implementations, the method 1800 may also include a step of fabricating a 3D physical instrument of a patient-specific instrument configured for use on the deformed osseous anatomy of the patient for the surgical procedure. Alternatively, or in addition, the method 1800 may also include a step of fabricating a 3D physical instrument of a patient-matched instrument configured for use on the deformed osseous anatomy of the patient for the surgical procedure.

Advantageously, patient-specific instrument and/or patient-matched instrument is configured for use with bones of a particular patient and/or group of patients. Thus, the patient-specific instrument and/or patient-matched instrument is ready for use in a surgical procedure on the patient. In addition, the patient-specific instrument and/or patient-matched instrument is configured for use with the physical bone models of a 3D physical bone model 1610 and/or 3D physical bone model 1620. Thus, a surgeon can use the same patient-specific instrument and/or patient-matched instrument for planning with the 3D physical bone model 1610 and/or 3D physical bone model 1620 as well as during the surgical procedure. This can be advantageous because with the 3D physical bone models a surgeon may identify a particular challenge or obstacle with using the patient-specific instrument and/or patient-matched instrument. Based on this discovery, the surgeon may then request a change in the patient-specific instrument and/or patient-matched instrument and/or may alter a plan for the surgical procedure to overcome the challenge or obstacle.

In a ninth implementation, alone or in combination with one or more of the first through eighth implementations, the patient-specific instrument may include a bone engagement member configured to engage a bone of the deformed osseous anatomy and to engage a physical bone model of the 3D physical bone model corresponding to the bone of the deformed osseous anatomy.

As described herein, the bone engagement member can assist a surgeon in placement and/or orientation of the patient-specific instrument during rehearsal or planning using the 3D physical bone models as well as during the surgical procedure.

Although FIG. 18 shows example steps of method 1800, in some implementations, method 1800 may include additional steps, fewer steps, different steps, or differently arranged steps than those depicted in FIG. 18. Additionally, or alternatively, two or more of the steps of method 1800 may be performed in parallel.

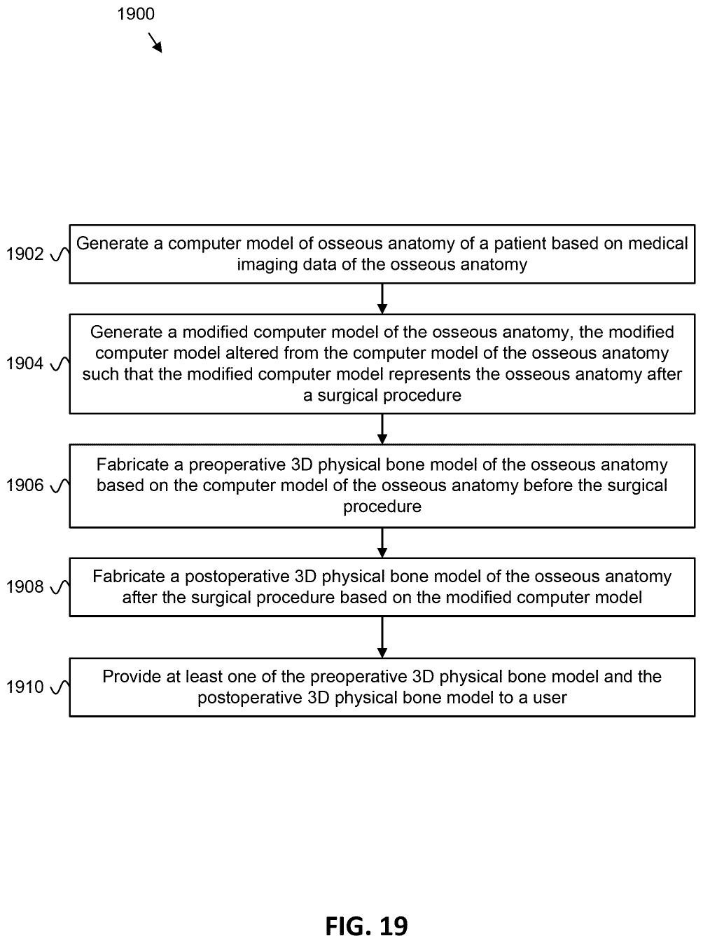

FIG. 19 is a flowchart of an example method 1900. In some implementations, one or more method steps of FIG. 19 may be performed by an apparatus, device, computing device, or system, such as for example system 800 or system 400.

As shown in FIG. 19, method 1900 may include generating a computer model of osseous anatomy of a patient based on medical imaging data of the osseous anatomy (step 1902). In another embodiment, the method 1900 may include either together or in place of step 1902, generating anatomic data 412 from medical imaging data taken of osseous anatomy of a patient and/or generating the computer model from the anatomic data 412. In certain embodiments, the medical imaging data is recent such that the osseous anatomy includes the patient's current condition.

In one embodiment, the osseous anatomy may represent bones of a patient in a deformed bone condition. Alternatively, or in addition, the osseous anatomy may represent bones of a patient in a healthy bone condition. In one embodiment, a patient and/or surgeon may use one or more methods, systems, and/or apparatuses of the present disclosure to assist in a surgical procedure on healthy anatomy. For example, a patient may desire one or more bones to be lengthened or shortened.

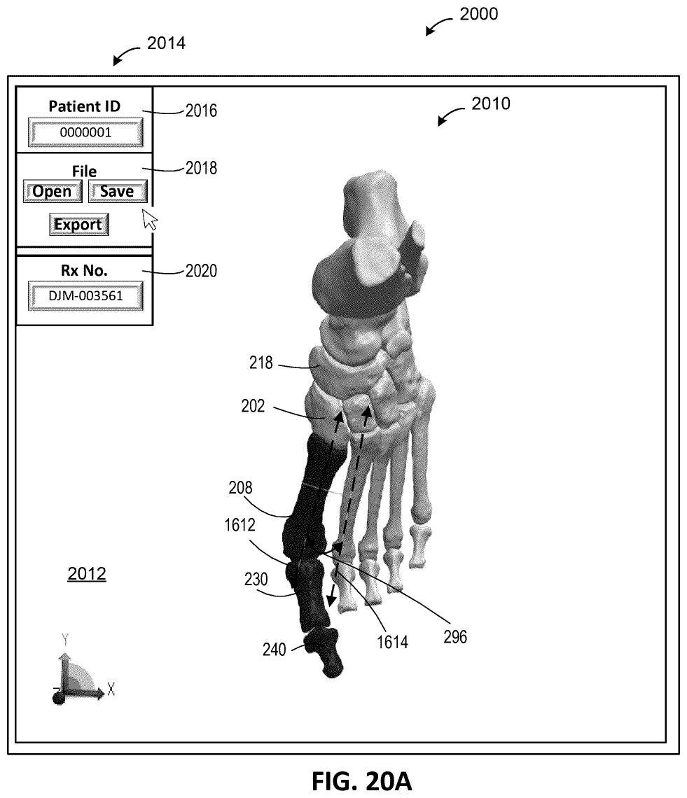

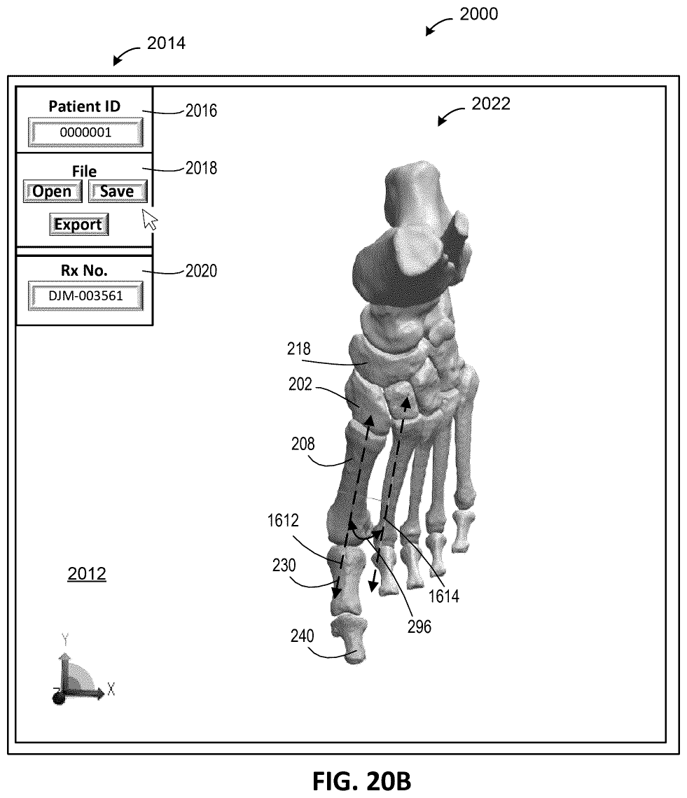

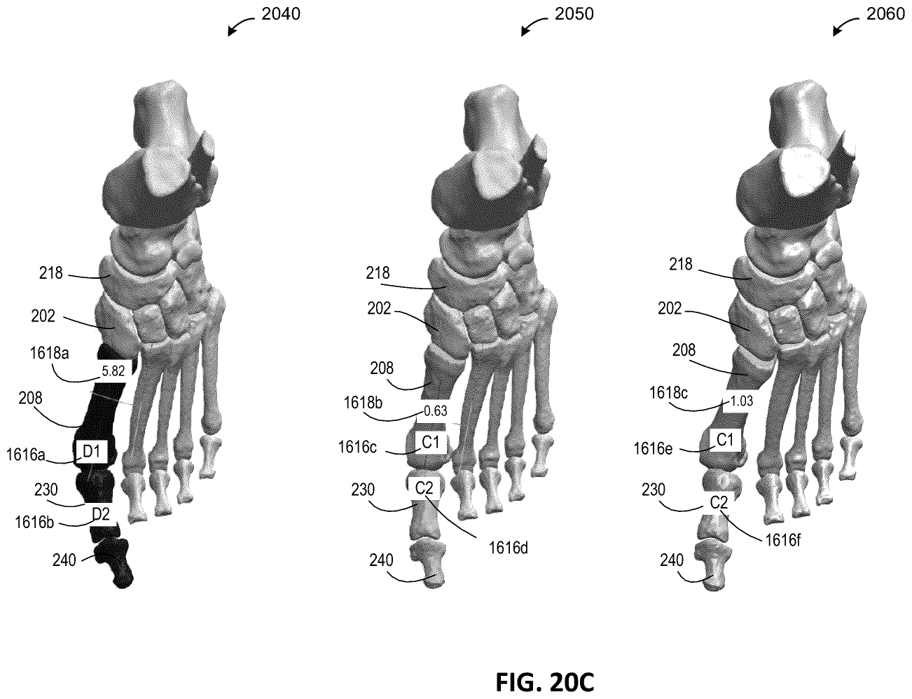

Referring now to FIG. 20A and FIG. 19, FIG. 20A illustrates an example user interface 2000 that presents a computer model 2010 of osseous anatomy on a three-dimensional environment pane 2012, according to one or more examples of the present disclosure. FIG. 20A illustrates an exemplary user-interface for working with a computer model for a surgical procedure, according to one embodiment.

The user interface 2000 may include the three-dimensional environment pane 2012 and a user input pane 2014. The user input pane 2014 may include a patient ID 2016 that shows a patient associated with the current computer model 2010. The user input pane 2014 may include a set of user input controls 2018 that enable a user to open, save, and export information for the current computer model 2010. The user input pane 2014 may include a prescription input box 2020 that enables a user to enter a prescription to be used with a current computer model 2010.

In one embodiment, the user interface 2000 may be presented to a user in response to a request or other user input. A user may initiate creation of the computer model 2010 or display of the computer model 2010 by entering a patient ID using the patient ID 2016 and/or the user input controls 2018. In one embodiment, the computer model 2010 is generated after a user indicates a location for medical imaging data and/or anatomic data 412. Alternatively, or in addition, the user interface 2000 may display or render a computer model 2010 that was generated in the past.

In certain embodiments, the computer model 2010 responds to user input via a keyboard, mouse, or touchscreen gestures. The user can rotate and/or translate the computer model 2010 on the three-dimensional environment pane 2012 and may be able to hide or display certain computer model bones of the computer model 2010 as the user desires. In one embodiment, the user interface 2000 may display the computer model 2010 in a dorsal anatomic position as illustrated in FIG. 20A by default. Advantageously, the user interface 2000 can be presented to a user on a variety of computing devices including but not limited to a mobile or handheld device, a laptop, a tablet, a desktop, and the like. User interface 2000 is one example, of course, the user interface 2000 may include a variety of other user input controls and/or features beyond those illustrated in this example.

In certain embodiments, the computer model 2010 may be presented with indicators that identify certain bones of deformed osseous anatomy (e.g., blue colored bones for bones that are in a deformed condition and tan colored bones for bones not in a deformed condition). Of course, other indicators can be used as well or in addition, such as shading, cross-hatching, letters, arrows, lines, etc.

In the illustrated embodiment, the user interface 2000 includes a first ray longitudinal axis 1612 and a second ray longitudinal axis 1614 and an intermetatarsal IM angle 296 measured between them. The user interface 2000 may report the measurement for the intermetatarsal IM angle 296 such that a user can compare this value to one for a modified computer model. The first ray longitudinal axis 1612, second ray longitudinal axis 1614, intermetatarsal IM angle 296 measurement may assist a user in diagnosing a condition and/or assessing a severity of a condition such as a deformity. Alternatively, or in addition, the user interface 2000 may report or display a value for the intermetatarsal IM angle 296. In one embodiment, the computer model 2010 represents a preoperative computer model of osseous anatomy of a patient. The three-dimensional model and nature of the computer model 2010 can provide valuable information to a surgeon.

In certain embodiments, the user interface 2000 may include a prescription input box 2020 that indicates a prescription that is to be used for design and/or development of a 3D physical bone model 1610, a 3D physical bone model 1620, and/or one or more instruments (e.g., patient-specific, patient-matched, or conventional). By entering a prescription number into the prescription input box 2020 a user can access the details of the prescription and/or may be able to apply the prescription to the computer model 2010 and/or to instrument models for use with the computer model 2010.

Referring back to FIG. 19, method 1900 may also include generating a modified computer model of the osseous anatomy, the modified computer model altered from the computer model of the osseous anatomy such that the modified computer model represents the osseous anatomy after a surgical procedure (step 1904). Those of skill in the art will appreciate that a modified computer model can be generated or provided in a variety of ways. First, a modified computer model can be generated from medical imaging and/or anatomic data 412 of osseous anatomy. Second, a modified computer model can be generated by altering a computer model 2010 of osseous anatomy. Third, a modified computer model can be generated based on a prescription from a doctor. In the illustrated embodiment, the modified computer model is modified such that the modified computer model represents the osseous anatomy after a surgical procedure.

Generating a modified computer model may be done by a computing device, a technician, a user, a patient, a doctor, a surgeon and/or a combination of these. In one embodiment, a computing device may create a preliminary modified computer model that is then refined and/or revised by a user, such as a technician and/or a doctor. For example, a computing device may create a preliminary modified computer model based on prescription or an algorithm or a determination made using artificial intelligence, or the like.

Method 1900 may include additional implementations, such as any single implementation or any combination of implementations described below and/or in connection with one or more other methods or processes described elsewhere herein. In a first implementation, generating a modified computer model further may include modifying the computer model based on a prescription provided by a surgeon. For example, the prescription may indicate a desired intermetatarsal IM angle 296 for the first metatarsal 208 relative to the second metatarsal 210. A computing device may initially reposition a first metatarsal 208 in a computer model 2010 to create a modified computer model 2022 that includes an intermetatarsal IM angle 296 as indicated in a prescription from a surgeon.

FIG. 20B illustrates an exemplary user-interface for working with a computer model for a surgical procedure, according to one embodiment. FIG. 20B represents the same user interface 2000 as FIG. 20A and displays a modified computer model 2022. The modified computer model 2022 includes the same model bones as the computer model 2010. Certain bones of the modified computer model 2022 have been repositioned to remediate a bone condition, for example to remediate a deformed bone condition of an osseous anatomy. In one embodiment, the deformed bone condition is a Bunion. Thus, the modified computer model 2022 is configured to remediate a deformed bone condition of osseous anatomy.

In certain embodiments, the modified computer model 2022 may be generated and/or created by a computing device with, or without, the aid of a user such as a technician and/or a surgeon. Alternatively, a user may create or generate the modified computer model 2022 by manipulating and/or revising a computer model 2010 and/or changing one or more of the medical imaging data and/or anatomic data 412.

In the illustrated embodiment of FIG. 20B, the user interface 2000 includes a first ray longitudinal axis 1612 and a second ray longitudinal axis 1614 and an intermetatarsal IM angle 296 measured between them. The user interface 2000 may report the measurement for the intermetatarsal IM angle 296 such that a user can compare this value to one for the computer model 2010. In one embodiment, a surgeon may review the modified computer model 2022 and the reported intermetatarsal IM angle 296 and determine if the new positioning is satisfactory. If not, the user may request further modifications and/or may perform the repositioning themselves using the user interface 2000.

As with the user interface 2000 of FIG. 20A, in FIG. 20B, the modified computer model 2022 may be presented with indicators that the identify certain bones of a deformed osseous anatomy that have been moved or changed to remediate a condition or accomplish a goal for a surgical procedure. (e.g., pink colored bones for bones that are in a corrected or repositioned condition and tan colored bones for bones in an original position). Of course, other indicators can be used as well or in addition, such as shading, cross-hatching, letters, arrows, lines, etc.

In certain embodiments, once a modified computer model 2022 is created, the modified computer model 2022 can be stored and preserved for review and/or use later. In addition, the modified computer model 2022 can be further modified as needed. Advantageously, in one embodiment, a user, such as a surgeon or technician, can modify the computer model 2010 and/or a version of the modified computer model 2022 to create another or a further modified computer model. In this manner, the user can create a plurality of modified computer model 2022 that each represent a different surgical technique and/or a different degree of repositioning for one or more of the bones of the computer model 2010. These plurality of modified computer model 2022 can be stored and referenced later by a surgeon as needed. For example, in one embodiment, a surgeon may request or may create a modified computer model 2022 for one or more stages of a surgical procedure.

Advantageously, the user interface 2000 enables a user such as a surgeon to modify the computer model 2010 and/or a modified computer model 2022 in response to user input. For example, a user may enter a desired value for an intermetatarsal IM angle 296, and a computing device may revise a modified computer model 2022 to reflect that desired measurement. Alternatively, or in addition, a user may use a mouse, keyboard, or touch gestures to further modify a computer model 2010 such that bones of the computer model 2010 are repositioned to form a modified computer model 2022 or a modified version of a modified computer model 2022.

The steps of modification and/or review may be repeated until a desired modified computer model 2022 is achieved. At this stage, a surgeon may approve of the modified computer model 2022 as well as any instruments (e.g., patient-specific or conventional) that may be prepared and/or planned for use with the modified computer model 2022.

As further shown in FIG. 19, method 1900 may include fabricating a preoperative 3D physical bone model of the osseous anatomy based on the computer model of the osseous anatomy before the surgical procedure (step 1906). FIG. 20C illustrates exemplary 3D physical bone models, according to one embodiment. FIG. 20C illustrates one example of a preoperative 3D physical bone model 2040. Note that the physical bone models of the preoperative 3D physical bone model 2040 are in the same positions as the computer model 2010. Like 3D physical bone model 1610, the preoperative 3D physical bone model 2040 may also include one or more indicators such as for example bone indicator 1616a D1, bone indicator 1616b D2, and angle indicator 1618a. Alternatively, or in addition, deformed model bones of the preoperative 3D physical bone model 2040 may be color coded (e.g., blue) to indicate which ones are in a deformed position (e.g., first metatarsal 208 and/or proximal phalange 230).

The preoperative 3D physical bone model 2040 provides a three-dimensional model that a user can handle, examine, rotate and/or manipulate to assist in planning a surgical procedure. In the illustrated embodiment, the preoperative 3D physical bone model 2040 is a 1:1 replica of bones of a patient.

As also shown in FIG. 19, method 1900 may include fabricating a postoperative 3D physical bone model of the osseous anatomy after the surgical procedure based on the modified computer model (step 1908). FIG. 20C illustrates one example of a postoperative 3D physical bone model 2050. Note that the physical bone models of the postoperative 3D physical bone model 2050 are in the same positions as the modified computer model 2022. Like 3D physical bone model 1620, the postoperative 3D physical bone model 2050 may also include one or more indicators such as for example bone indicator 1616c C1, bone indicator 1616d C2, and angle indicator 1618b. Alternatively, or in addition, repositioned and/or corrected model bones of the postoperative 3D physical bone model 2050 may be color coded (e.g., pink) to indicate which ones are in a corrected or repositioned position (e.g., first metatarsal 208 and/or proximal phalange 230).

The postoperative 3D physical bone model 2050 provides a three-dimensional model that a user can handle, examine, rotate and/or manipulate to assist in planning a surgical procedure. In the illustrated embodiment, the postoperative 3D physical bone model 2050 is a 1:1 replica of bones of a patient. In certain embodiments, the postoperative 3D physical bone model 2050 may include model bones that include cut faces on them to model where planned cuts are to be made during a surgical procedure. For example, in postoperative 3D physical bone model 2050 a distal end of a medial cuneiform 202 may include a planar cut face and a proximal end of a first metatarsal 208 may include a planar cut face. In the postoperative 3D physical bone model 2050 the two cut faces may abut each other and the postoperative 3D physical bone model 2050 may include a model fastener such as a bone plate and/or one or more bone screws and/or a bone staple positioned as planned in a preoperative plan. In certain embodiments, the fasteners may be removable such that a surgeon can trial different fasteners. In one embodiment, the fasteners are models and in another embodiment the fasteners are fasteners that are to be used in the surgical procedure. Other osteotomies for the patient may also be modeled in a postoperative 3D physical bone model 2050 such as a closing wedge osteotomy for an Akin procedure, and the like.

As also shown in FIG. 19, method 1900 may include providing at least one of the preoperative 3D physical bone model and the postoperative 3D physical bone model to a user (step 1910). In certain embodiments, the method 1900 may provide both a preoperative 3D physical bone model 2040 and a postoperative 3D physical bone model 2050 to a user. Thus, a user may receive two 3D physical models a preoperative 3D physical bone model 2040 and a postoperative 3D physical bone model 2050.

In one embodiment, at least one of the preoperative 3D physical bone model 2040 and the postoperative 3D physical bone model 2050 may include an indicator that distinguishes the preoperative 3D physical bone model 2040 from a postoperative 3D physical bone model 2050. In one embodiment, the indicator may be printed, embossed, debossed, or the like on the model 2040, 2050. For example, the text “preoperative” may be placed on the preoperative 3D physical bone model 2040 and the text “postoperative” may be placed on the postoperative 3D physical bone model 2050.

In addition, the models 2040,2050 may include indicators such as angle indicator 1618a and angle indicator 1618. Having both a preoperative 3D physical bone model 2040 and a postoperative 3D physical bone model 2050 can assist a surgeon in confirming that their surgical procedure plan is appropriate and will provide the highest likelihood of achieving a desired outcome.

In addition, the angle indicator 1618a may include an IM measurement, for example 5.82 degrees and the angle indicator 1618b may include an IM measurement, for example 0.63 degrees. A surgeon can compare these measurements for each of the preoperative 3D physical bone model 2040 and the postoperative 3D physical bone model 2050 and determine if the change in angle is the amount desired and/or required. Furthermore, a surgeon can handle and/or may be able to manipulate physical bone models of one or more of the preoperative 3D physical bone model 2040 and the postoperative 3D physical bone model 2050 and further confirm and/or validate a preoperative plan.

In certain embodiments, a surgeon may examine the preoperative 3D physical bone model 2040 and the postoperative 3D physical bone model 2050 and determine that a change is needed to the preoperative plan and/or the prescription. In such an instance, a surgeon may revise a computer model 2010 and generate a new modified computer model 2022 that can then be used to generate a revised postoperative 3D physical bone model 2050. The surgeon may then compare the revised postoperative 3D physical bone model 2050 to the preoperative 3D physical bone model 2040 and confirm that the additional change is satisfactory.

As shown in FIG. 19, method 1900 may include generating a computer model of osseous anatomy of a patient based on medical imaging data of the osseous anatomy (step 1902). As also shown in FIG. 19, FIG. 19, method 1900 may also include generating a modified computer model of the osseous anatomy, the modified computer model altered from the computer model of the osseous anatomy such that the modified computer model represents the osseous anatomy after a surgical procedure (step 1904). As further shown in FIG. 19, method 1900 may include fabricating a preoperative 3D physical bone model of the osseous anatomy based on the computer model of the osseous anatomy before the surgical procedure (step 1906). As also shown in FIG. 19, method 1900 may include fabricating a postoperative 3D physical bone model of the osseous anatomy after the surgical procedure based on the modified computer model (step 1908). As also shown in FIG. 19, method 1900 may include providing at least one of the preoperative 3D physical bone model and the postoperative 3D physical bone model to a user (step 1910).

Method 1900 may include additional implementations, such as any single implementation or any combination of implementations described below and/or in connection with one or more other methods or processes described elsewhere herein. In a first implementation, generating a modified computer model further may include modifying the computer model based on a prescription provided by a surgeon.

In a second implementation, alone or in combination with the first implementation, the method 1900 may include generating a modified computer model further may include modifying the computer model in response to user input.

In a third implementation, the method 1900 may include alone or in combination with the first and second implementation, providing an indicator on one of the preoperative 3D physical bone model and the postoperative 3D physical bone model that distinguishes the preoperative 3D physical bone model from the postoperative 3D physical bone model.

In a fourth implementation, alone or in combination with one or more of the first through third implementations, the method 1900 may include wherein the modified computer model is configured to remediate a deformed bone condition of the osseous anatomy.

In a fifth implementation, alone or in combination with one or more of the first through fourth implementations, the method 1900 may include fabricating a second postoperative 3D physical bone model based on the computer model of the osseous anatomy. FIG. 20C illustrates one example of a second postoperative 3D physical bone model 2060. In certain embodiments, a surgeon may want a second postoperative 3D physical bone model 2060 as well as a postoperative 3D physical bone model 2050 because the second postoperative 3D physical bone model 2060 may represent an alternative direction for the surgical procedure. For example, in the illustrated embodiment, the second postoperative 3D physical bone model 2060 may represent a smaller change in the intermetatarsal IM angle 296 (e.g., from 5.82 degrees to 1.03 degrees). Like the postoperative 3D physical bone model 2050 the second postoperative 3D physical bone model 2060 may include one or more indicators for the benefit of the user including bone indicator 1616e, bone indicator 1616f, angle indicator 1618c that shows the postoperative IM angle.

Alternatively, or in addition, the second postoperative 3D physical bone model 2060 may represent one or more bones at stage within a surgical procedure. This version of a second postoperative 3D physical bone model 2060 may be useful to a surgeon to understand how the anatomy may change during a surgical procedure. In yet another embodiment, the second postoperative 3D physical bone model 2060 may represent a patient's anatomy with the bones in a configuration other than postoperative such as a plantarflexed position or dorsal flexed position or any other configuration, such as the examples included in this disclosure.

Although FIG. 19 shows example steps of method 1900, in some implementations, method 1900 may include additional steps, fewer steps, different steps, or differently arranged steps than those depicted in FIG. 19. Additionally, or alternatively, two or more of the steps of method 1900 may be performed in parallel.

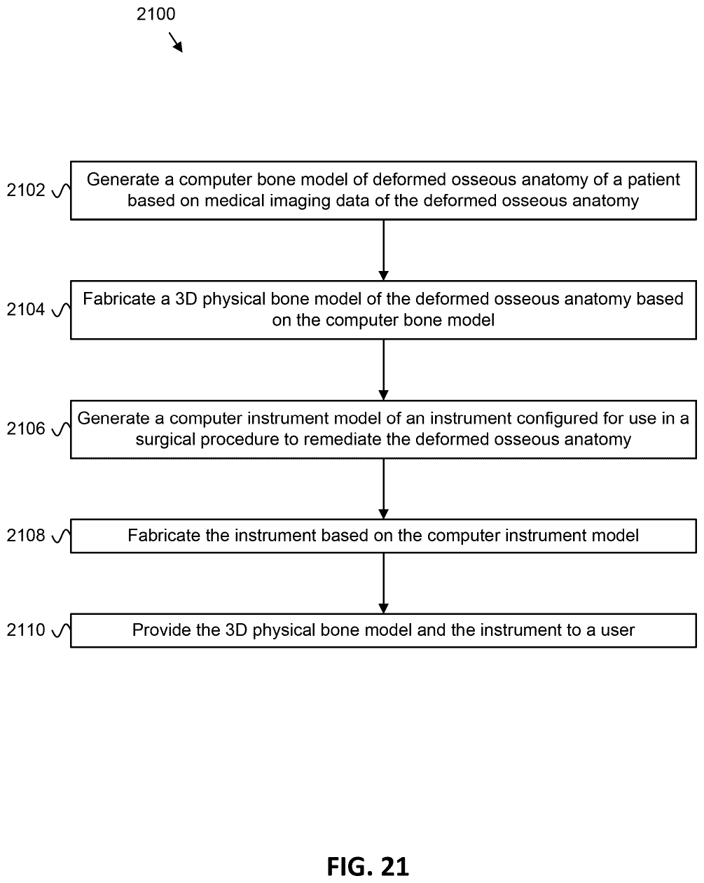

FIG. 21 illustrates a method for providing a 3D physical model for a surgical procedure, according to one embodiment. FIG. 21 is a flowchart of an example method 2100. In some implementations, one or more method steps of FIG. 21 may be performed by an apparatus, device, computing device, or system, such as for example system 800, system 400, or the like.

As shown in FIG. 21, method 2100 may include generating a computer bone model of deformed osseous anatomy of a patient based on medical imaging data of the deformed osseous anatomy (step 2102). As also shown in FIG. 21, method 2100 may include fabricating a 3D physical bone model of the deformed osseous anatomy based on the computer bone model (step 2104). As further shown in FIG. 21, method 2100 may include generating a computer instrument model of an instrument configured for use in a surgical procedure to remediate the deformed osseous anatomy (step 2106). As also shown in FIG. 21, method 2100 may include fabricating the instrument based on the computer instrument model (step 2108). As further shown in FIG. 21, method 2100 may include providing the 3D physical bone model and the instrument to a user (step 2110).

Method 2100 may include additional implementations, such as any single implementation or any combination of implementations described below and/or in connection with one or more other methods or processes described elsewhere herein. In a first implementation, the instrument is a patient-specific instrument having a bone engagement member configured to engage a bone of the deformed osseous anatomy and to engage a physical bone model of the 3D physical bone model corresponding to the bone of the deformed osseous anatomy.

Although FIG. 21 shows example steps of method 2100, in some implementations, method 2100 may include additional steps, fewer steps, different steps, or differently arranged steps than those depicted in FIG. 21. Additionally, or alternatively, two or more of the steps of method 2100 may be performed in parallel.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Alternatively, or in addition, any of the systems, devices, and/or apparatuses herein can be implemented using fewer than the elements and/or components described in the presented embodiments. In addition, components and/or elements and/or structures in one embodiment may be used in other embodiments to replace a component or structure and/or to augment the embodiment within the scope of the claims and present disclosure.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the methods and systems of the present disclosure set forth herein without departing from it spirit and scope.

What is claimed is:

1. A method for providing a three-dimensional (3D) physical model for a surgical procedure, the method comprising:
generating a computer model of deformed osseous anatomy of a patient based on medical imaging data of the deformed osseous anatomy;
fabricating a 3D physical bone model of the deformed osseous anatomy based on the computer model; and
providing the 3D physical bone model to a user.

2. The method of claim 1, wherein the deformed osseous anatomy comprises a plurality of bones of the patient and wherein fabricating a 3D physical bone model comprises coupling two physical bone models of the 3D physical bone model by way of an interconnect such that the two physical bone models connect in correspondence with patient bones of the deformed osseous anatomy.

3. The method of claim 2, wherein the interconnect comprises an interface configured to enable a user to reposition one physical bone model in relation to another physical bone model coupled to the interface.

4. The method of claim 2, wherein the interconnect comprises a rigid interconnect.

5. The method of claim 2, wherein the interconnect comprises a detachable interconnect.

6. The method of claim 2, wherein fabricating a 3D physical bone model comprises coupling a plurality of physical bone models of the 3D physical bone model by way of a plurality of interconnects and wherein the plurality of interconnects comprises rigid interconnects and adjustable interconnects.

7. The method of claim 1, wherein fabricating a 3D physical bone model comprises providing an indicator on a physical bone model of the 3D physical bone model, the indicator configured to convey information about at least one of the osseous anatomy, the patient, and the surgical procedure.

8. The method of claim 7, wherein the indicator identifies a physical bone model corresponding to a patient bone having a deformed bone condition.

9. The method of claim 1, comprising fabricating a 3D physical instrument of a patient-specific instrument configured for use on the deformed osseous anatomy of the patient for the surgical procedure.

10. The method of claim 9, wherein the 3D physical instrument comprises a bone engagement member configured to engage a bone of the deformed osseous anatomy and to engage a physical bone model of the 3D physical bone model corresponding to the bone of the deformed osseous anatomy.

11. A method for providing a three-dimensional (3D) physical model for a surgical procedure, the method comprising:
generating a computer model of osseous anatomy of a patient based on medical imaging data of the osseous anatomy;
generating a modified computer model of the osseous anatomy, the modified computer model altered from the computer model of the osseous anatomy such that the modified computer model represents the osseous anatomy after a surgical procedure;
fabricating a preoperative 3D physical bone model of the osseous anatomy based on the computer model of the osseous anatomy before the surgical procedure;
fabricating a postoperative 3D physical bone model of the osseous anatomy after the surgical procedure based on the modified computer model;
and
providing at least one of the preoperative 3D physical bone model and the postoperative 3D physical bone model to a user.

12. The method of claim 11, wherein generating a modified computer model further comprises modifying the computer model based on a prescription provided by a surgeon.

13. The method of claim 11, wherein generating a modified computer model further comprises modifying the computer model in response to user input.

14. The method of claim 11, comprising providing an indicator on one of the preoperative 3D physical bone model and the postoperative 3D physical bone model that distinguishes the preoperative 3D physical bone model from the postoperative 3D physical bone model.

15. The method of claim 11, wherein the modified computer model is configured to remediate a deformed bone condition of the osseous anatomy.

16. The method of claim 11, comprising fabricating a second postoperative 3D physical bone model based on the computer model of the osseous anatomy.

17. A method for providing a three-dimensional (3D) physical model for a surgical procedure, the method comprising:
generating a computer bone model of deformed osseous anatomy of a patient based on medical imaging data of the deformed osseous anatomy;
fabricating a 3D physical bone model of the deformed osseous anatomy based on the computer bone model;
generating a computer instrument model of an instrument configured for use in a surgical procedure to remediate the deformed osseous anatomy;
fabricating the instrument based on the computer instrument model; and
providing the 3D physical bone model and the instrument to a user.

18. The method of claim 17, wherein the instrument is a patient-specific instrument comprising a bone engagement member configured to engage a bone of the deformed osseous anatomy and to engage a physical bone model of the 3D physical bone model corresponding to the bone of the deformed osseous anatomy.

19. A kit for an osteotomy procedure comprising:
a first 3D physical bone model of deformed osseous anatomy of a patient for an osteotomy procedure;
a preoperative plan for the osteotomy procedure; and
a set of patient-specific instruments for one or more stages of the osteotomy procedure.

20. The kit of claim 19, further comprising a second 3D physical bone model comprising a plurality of physical bone models coupled by way of a plurality of interconnects, the plurality of physical bone models coupled according to a predetermined configuration.

21. The kit of claim 20, further wherein the predetermined configuration is selected from the group comprising an anatomical position, a normal position, a weight bearing position, one stage of a walking position, one stage of a running position, one stage of a jumping position, one stage of a grasping position, a dorsiflexed position, a plantar flexed position, a laterally rotated position, a medially rotated position, an eversion rotated position, an inversion rotated position, a deformed condition, a corrected condition, a preoperative condition, and a postoperative condition.
